SEQUENCE LISTING (99 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 223 aa, synthetic construct
  REGION 1..223: note = Synthetic Construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 3 - protein, 32 aa, synthetic construct
  REGION 1..32: note = Synthetic Construct
GSGYIPEAPRDGQAYVRKDGEWVLLSTFLGRS

SEQ ID NO: 4 - protein, 33 aa, synthetic construct
  REGION 1..33: note = Synthetic Construct
RMKQIEDKIEEILSKIYHIENEIARIKKLIGER

SEQ ID NO: 5 - protein, 10 aa, synthetic construct
  REGION 1..10: note = Synthetic Construct
GGGGSGGGGS

SEQ ID NO: 6 - protein, 588 aa, SARS-CoV-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 7 - protein, 588 aa, synthetic construct
  REGION 1..588: note = Synthetic Construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1272 aa, SARS-CoV-2
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 1268 aa, synthetic construct
  REGION 1..1268: note = Synthetic Construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 10 - protein, 1268 aa, synthetic construct
  REGION 1..1268: note = Synthetic Construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSRSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - RNA, 1764 nt, SARS-CoV-2
  source 1..1764: mol_type = unassigned RNA
agtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 12 - RNA, 1764 nt, synthetic construct
  source 1..1764: mol_type = other RNA
agtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgatcctccagaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 13 - RNA, 1764 nt, SARS-CoV-2; identical to SEQ ID NO: 12
  source 1..1764: mol_type = unassigned RNA

SEQ ID NO: 14 - RNA, 3804 nt, synthetic construct
  misc_feature 1..3804: note = Synthetic Construct
  source 1..3804: mol_type = other RNA
ttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 15 - RNA, 3804 nt, synthetic construct
  misc_feature 1..3804: note = Synthetic Construct
  source 1..3804: mol_type = other RNA
ttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgatcctccagaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 16 - protein, 31 aa, synthetic construct
  REGION 1..31: note = Synthetic Construct
DAMKRGLCCVLLLCGAVFVSPSQEIHARFRR

SEQ ID NO: 17 - protein, 17 aa, Homo sapiens
DWTWILFLVAAATRVHS

SEQ ID NO: 18 - protein, 634 aa, Mus musculus
MLTFFAAFLAAPLALAESPYLVRVDAARPLRPLLPFWRSTGFCPPLPHDQADQYDLSWDQQLNLAYIGAVPHSGIEQVRIHWLLDLITARKSPGQGLMYNFTHLDAFLDLLMENQLLPGFELMGSPSGYFTDFDDKQQVFEWKDLVSLLARRYIGRYGLTHVSKWNFETWNEPDHHDFDNVSMTTQGFLNYYDACSEGLRIASPTLKLGGPGDSFHPLPRSPMCWSLLGHCANGTNFFTGEVGVRLDYISLHKKGAGSSIAILEQEMAVVEQVQQLFPEFKDTPIYNDEADPLVGWSLPQPWRADVTYAALVVKVIAQHQNLLFANSSSSMRYVLLSNDNAFLSYHPYPFSQRTLTARFQVNNTHPPHVQLLRKPVLTVMGLMALLDGEQLWAEVSKAGAVLDSNHTVGVLASTHHPEGSAAAWSTTVLIYTSDDTHAHPNHSIPVTLRLRGVPPGLDLVYIVLYLDNQLSSPYSAWQHMGQPVFPSAEQFRRMRMVEDPVAEAPRPFPARGRLTLHRKLPVPSLLLVHVCTRPLKPPGQVSRLRALPLTHGQLILVWSDERVGSKCLWTYEIQFSQKGEEYAPINRRPSTFNLFVFSPDTAVVSGSYRVRALDYWARPGPFSDPVTYLDVPAS

SEQ ID NO: 19 - protein, 653 aa, Homo sapiens
MRPLRPRAALLALLASLLAAPPVAPAEAPHLVHVDAARALWPLRRFWRSTGFCPPLPHSQADQYVLSWDQQLNLAYVGAVPHRGIKQVRTHWLLELVTTRGSTGRGLSYNFTHLDGYLDLLRENQLLPGFELMGSASGHFTDFEDKQQVFEWKDLVSSLARRYIGRYGLAHVSKWNFETWNEPDHHDFDNVSMTMQGFLNYYDACSEGLRAASPALRLGGPGDSFHTPPRSPLSWGLLRHCHDGTNFFTGEAGVRLDYISLHRKGARSSISILEQEKVVAQQIRQLFPKFADTPIYNDEADPLVGWSLPQPWRADVTYAAMVVKVIAQHQNLLLANTTSAFPYALLSNDNAFLSYHPHPFAQRTLTARFQVNNTRPPHVQLLRKPVLTAMGLLALLDEEQLWAEVSQAGTVLDSNHTVGVLASAHRPQGPADAWRAAVLIYASDDTRAHPNRSVAVTLRLRGVPPGPGLVYVTRYLDNGLCSPDGEWRRLGRPVFPTAEQFRRMRAAEDPVAAAPRPLPAGGRLTLRPALRLPSLLLVHVCARPEKPPGQVTRLRALPLTQGQLVLVWSDEHVGSKCLWTYEIQFSQDGKAYTPVSRKPSTFNLFVFSPDTGAVSGSYRVRALDYWARPGPFSDPVPYLEVPVPRGPPSPGNP

SEQ ID NO: 20 - protein, 354 aa, Mus musculus
MLSNLRILLNNAALRKGHTSVVRHFWCGKPVQSQVQLKGRDLLTLKNFTGEEIQYMLWLSADLKFRIKQKGEYLPLLQGKSLGMIFEKRSTRTRLSTETGFALLGGHPSFLTTQDIHLGVNESLTDTARVLSSMTDAVLARVYKQSDLDTLAKEASIPIVNGLSDLYHPIQILADYLTLQEHYGSLKGLTLSWIGDGNNILHSIMMSAAKFGMHLQAATPKGYEPDPNIVKLAEQYAKENGTKLSMTNDPLEAARGGNVLITDTWISMGQEDEKKKRLQAFQGYQVTMKTAKVAASDWTFLHCLPRKPEEVDDEVFYSPRSLVFPEAENRKWTIMAVMVSLLTDYSPVLQKPKF

SEQ ID NO: 21 - protein, 419 aa, Mus musculus
MSFIPVAEDSDFPIQNLPYGVFSTQSNPKPRIGVAIGDQILDLSVIKHLFTGPALSKHQHVFDETTLNNFMGLGQAAWKEARASLQNLLSASQARLRDDKELRQRAFTSQASATMHLPATIGDYTDFYSSRQHATNVGIMFRGKENALLPNWLHLPVGYHGRASSIVVSGTPIRRPMGQMRPDNSKPPVYGACRLLDMELEMAFFVGPGNRFGEPIPISKAHEHIFGMVLMNDWSARDIQQWEYVPLGPFLGKSFGTTISPWVVPMDALMPFVVPNPKQDPKPLPYLCHSQPYTFDINLSVSLKGEGMSQAATICRSNFKHMYWTMLQQLTHHSVNGCNLRPGDLLASGTISGSDPESFGSMLELSWKGTKAIDVEQGQTRTFLLDGDEVIITGHCQGDGYRVGFGQCAGKVLPALSPA

SEQ ID NO: 22 - protein, 1983 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 23 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLRELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 24 - protein, 393 aa, Homo sapiens [sequence omitted]

SEQ ID NO: 25 - protein, 403 aa, Homo sapiens [sequence omitted]

SEQ ID NO: 26 - protein, 117 aa, SARS-CoV-2 [sequence omitted]

SEQ ID NO: 27 - protein, 117 aa, SARS-CoV-2
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 28 - protein, 403 aa, SARS-CoV-2
QVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSKLGGGGSGGGGSGGGGSGGGGSGGGGSQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSSGGGGSGGGGSGGGGSGGGGSGGGGSQVQLVESGGGLVQAGGSLRLSCAVSGAGAHRVGWFRRAPGKEREFVAAIGASGGMTNYLDSVKGRFTISRDNAKNTIYLQMNSLKPQDTAVYYCAARDIETAEYIYWGQGTQVTVSS

SEQ ID NO: 29 - protein, 404 aa, SARS-CoV-2
QVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSKLGGGGSGGGGSGGGGSGGGGSGGGGSSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSSGGGGSGGGGSGGGGSGGGGSGGGGSQVQLVESGGGLVQAGGSLRLSCAVSGLGAHRVGWFRRAPGKEREFVAAIGANGGNTNYLDSVKGRFTISRDNAKNTIYLQMNSLKPQDTAVYYCAARDIETAEYTYWGQGTQVTVSS

SEQ ID NO: 30 - protein, 119 aa, SARS-CoV-2
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSS

SEQ ID NO: 31 - protein, 397 aa, SARS-CoV-2
QVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSSGGGGSGGGGSGGGGSGGGGSQVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSSGGGGSGGGGSGGGGSGGGGSQVQLVESGGGLVQAGGSLRLSCAASGYIFGRNAMGWYRQAPGKERELVAGITRRGSITYYADSVKGRFTISRDNAKNTVYLQMNSLKPEDTAVYYCAADPASPAYGDYWGQGTQVTVSS

SEQ ID NO: 32 - protein, 455 aa, SARS-CoV-2
EVQLLESGGGVVQPGGSLRLSCAASGFAFTTYAMNWVRQAPGRGLEWVSAISDGGGSAYYADSVKGRFTISRDNSKNTLYLQMNSLRAEDTAVYYCAKTRGRGLYDYVWGSKDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 219 aa, SARS-CoV-2
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPGTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 167 aa, synthetic construct
  REGION 1..167: note = Synthetic Construct
SAEIDLGKGDFREIRASEDAREAAEALAEAARAMKEALEIIREIAEKLRDSSRASEAAKRIAKAIRKAADAIAEAAKIAARAAKDGDAARNAENAARKAKEFAEEQAKLADMYAELAKNGDKSSVLEQLKTFADKAFHEMEDRFYQAALAVFEAAEAAAGGSGWGSG

SEQ ID NO: 35 - protein, 344 aa, synthetic construct
  REGION 1..344: note = Synthetic Construct
SAEIDLGKGDFREIRASEDAREAAEALAEAARAMKEALEIIREIAEKLRDSSRASEAAKRIAKAIRKAADAIAEAAKIAARAAKDGDAARNAENAARKAKEFAEEQAKLADMYAELAKNGDKSSVLEQLKTFADKAFHEMEDRFYQAALAVFEAAEAAAGGGGSGGSGSGGSGGGSPGSAEIDLGKGDFREIRASEDAREAAEALAEAARAMKEALEIIREIAEKLRDSSRASEAAKRIAKAIRKAADAIAEAAKIAARAAKDGDAARNAENAARKAKEFAEEQAKLADMYAELAKNGDKSSVLEQLKTFADKAFHEMEDRFYQAALAVFEAAEAAAGGSGWGS

SEQ ID NO: 36 - skipped

SEQ ID NO: 37 - RNA, 51 nt, synthetic construct
  misc_feature 1..51: note = Synthetic Construct
  source 1..51: mol_type = other RNA
gaaaaacaaaaaacaaaaaaaacaaaaaaaaaaccaaaaaaacaaaacaca

SEQ ID NO: 38 - RNA, 27 nt, synthetic construct
  misc_feature 1..27: note = Synthetic Construct
  source 1..27: mol_type = other RNA
acgagtcctggactgaaacggacttgt

SEQ ID NO: 39 - RNA, 52 nt, synthetic construct
  misc_feature 1..52: note = Synthetic Construct
  source 1..52: mol_type = other RNA
aaaatccgttgaccttaaacggtcgtgtgggttcaagtccctccacccccac

SEQ ID NO: 40 - RNA, 16 nt, synthetic construct
  misc_feature 1..16: note = Synthetic Construct
  source 1..16: mol_type = other RNA
gagacgctacggactt

SEQ ID NO: 41 - RNA, 33 nt, synthetic construct
  misc_feature 1..33: note = Synthetic Construct
  source 1..33: mol_type = other RNA
gggagaccctcgaccgtcgattgtccactggtc

SEQ ID NO: 42 - RNA, 35 nt, synthetic construct
  misc_feature 1..35: note = Synthetic Construct
  source 1..35: mol_type = other RNA
accagtggacaatcgacggataacagcatatctag

SEQ ID NO: 43 - RNA, 19 nt, synthetic construct
  misc_feature 1..19: note = Synthetic Construct
  source 1..19: mol_type = other RNA
taatacgactcactatagg

SEQ ID NO: 44 - RNA, 54 nt, synthetic construct
  misc_feature 1..54: note = Synthetic Construct
  source 1..54: mol_type = other RNA
gagggcagaggaagtcttctaacatgcggtgacgtggaggagaatcccggccct

SEQ ID NO: 45 - RNA, 57 nt, synthetic construct
  misc_feature 1..57: note = Synthetic Construct
  source 1..57: mol_type = other RNA
gctactaacttcagcctgctgaagcaggctggagacgtggaggagaaccctggacct

SEQ ID NO: 46 - RNA, 131 nt, synthetic construct
  misc_feature 1..131: note = Synthetic Construct
  source 1..131: mol_type = other RNA
aacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 47 - RNA, 116 nt, synthetic construct
  misc_feature 1..116: note = Synthetic Construct
  source 1..116: mol_type = other RNA
aaataattgagccttaaagaagaaattctttaagtggatgctctcaaactcagggaaacctaaatctagttatagacaaggcaatcctgagccaagccgaagtagtaattagtaag

SEQ ID NO: 48 - RNA, 3819 nt, SARS-CoV-2
  source 1..3819: mol_type = unassigned RNA
atgttcgttttccttgttctgttgcctctcgttagtagccaatgcgtcaaccttactactagaacccagctccctccagcatataccaactctttcaccaggggcgtatattacccggacaaagtgttccgctcaagtgtgctgcattctacgcaggaccttttcttgccctttttcagtaatgttacttggtttcatgctatccatgtgtctggaactaacggaaccaagcgctttgacaaccccgtcctccctttcaacgatggcgtgtacttcgcttccacggaaaagtcaaacataattcgcggctggatctttggtacaacactcgactcaaagacgcagagcctgctgatcgttaataacgctacaaatgttgtgataaaggtgtgtgaatttcagttctgcaatgatcccttcctgggtgtgtactaccataagaataacaagagctggatggaatccgaatttagggtttacagttccgctaacaactgcacattcgaatacgtaagccagccatttcttatggatcttgagggcaagcaaggaaacttcaagaacttgagggagttcgtgttcaaaaatatcgacggctattttaagatatatagcaagcacactccaataaacttggtgcgcgacctgccccagggattctctgctctggagcccctggtggatctgcccattggaataaacataactcgctttcaaacactgctcgccctgcatcgcagttacctcacccctggtgatagtagttcaggatggacagcaggagccgccgcatactacgtcggctacctgcagcctaggaccttcttgctgaagtacaacgagaacggtacaataactgacgctgtggactgcgctctggaccctctgtccgagacgaagtgcaccctgaagagctttactgttgaaaaaggcatttaccaaaccagcaacttccgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttcaatttcaatggcctcactggaactggagtgctgaccgaatccaataagaagttcttgcccttccagcagttcggaagagacattgctgacacaaccgacgcggtgcgcgatcctcagactctggagatattggacattacaccatgttctttcggcggtgtgtctgtcattactccgggcacgaatactagcaaccaggtagccgtgctgtaccaagacgtgaattgcacagaggttcccgtcgcaattcacgctgaccagctgacccccacgtggagggtttacagcactggtagtaacgtcttccagacgagagccggttgcttgatcggagcggaacatgtgaataactcctacgagtgcgacatccccatcggagccggtatatgcgcctcttatcagacacaaactaactcacccaggagagcccgcagtgtggcttctcaaagcattatagcatacactatgtctcttggtgccgaaaattccgtggcctattctaacaattcaatcgccatcccaaccaacttcacaattagcgtgactaccgaaatactgcctgtgagcatgacgaaaaccagcgtagactgcactatgtatatctgtggagactccactgagtgctccaaccttctcctgcagtacggtagcttctgtacccaattgaaccgcgcccttacaggcatcgctgttgagcaagataagaatacccaggaagtttttgcccaggttaagcagatatacaaaacaccgcccattaaggacttcggaggcttcaacttctctcagatactgcctgacccctccaagccatcaaaacgcagcttcattgaggacctcttgttcaacaaagtgactctggctgatgctggcttcattaagcagtacggagattgcctgggagatattgctgccagggacctcatctgcgcccagaagtttaatggcctgacagtcttgcccccacttctgacagacgagatgattgctcagtacacatctgccctcctcgctggcaccataacatccggatggacatttggtgctggtgctgccctccagattcccttcgcaatgcagatggcgtatcgctttaacggcatcggtgtcacacaaaacgtgttgtatgagaaccaaaagctcatcgctaaccagtttaattctgctattggtaagattcaggacagcctgtcatcaaccgcgtctgcccttggtaagttgcaggacgtggtgaaccagaatgctcaggctttgaatactctggtgaagcaactctcttcaaatttcggcgctatctcttctgtgttgaacgacatcctgagtcgccttgataaggtggaagctgaagttcaaattgatagattgattactggcaggctccagtctttgcagacctacgttacacagcagctgattagggcggctgaaattagagcttccgccaatctggctgcaaccaagatgtccgaatgcgtcctgggtcagtcaaagcgcgttgacttttgtggtaaaggctaccacctcatgtcatttccccagtcagcacctcacggagtagtgttcctccacgtcacctacgttccagcacaggaaaagaattttaccactgcgccggcaatctgtcacgacggtaaggcacacttcccccgcgagggcgtattcgtgtctaacggaactcattggttcgtcacacagagaaacttctatgagcctcagatcattaccaccgacaatacatttgtgtccggtaactgcgacgttgtgattggaatcgtcaacaacactgtgtacgatccacttcagccagaactggatagcttcaaggaagaattggacaaatatttcaaaaatcacacttcacccgatgtggacctgggtgacattagtggtatcaatgcgtccgtggtcaatattcaaaaagagattgacaggctcaacgaagtggccaagaacctgaacgaaagtcttatcgatctgcaagaattgggaaagtatgagcagtacatcaagtggccgtggtacatttggttgggttttatcgccggtctgatcgccatcgttatggttaccattatgctttgctgcatgacgagctgttgctcctgtctgaagggatgctgctcttgcggatcatgttgcaagttcgatgaagacgatagcgaaccagttctgaagggcgtcaagctgcattacaca

SEQ ID NO: 49 - RNA, 669 nt, synthetic construct
  misc_feature 1..669: note = Synthetic Construct
  source 1..669: mol_type = other RNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 50 - RNA, 96 nt, synthetic construct
  misc_feature 1..96: note = Synthetic Construct
  source 1..96: mol_type = other RNA
ggaagcggctacatcccagaagcccctagagacggacaggcttacgtgcgaaaagacggcgagtgggtgctgctgagcacattcctgggaaggagc

SEQ ID NO: 51 - RNA, 99 nt, synthetic construct
  misc_feature 1..99: note = Synthetic Construct
  source 1..99: mol_type = other RNA
cgaatgaagcagattgaggataaaattgaggagattctcagcaaaatttaccacatagaaaatgagatcgctcggattaaaaaactgatcggagaaaga

SEQ ID NO: 52 - RNA, 30 nt, synthetic construct
  misc_feature 1..30: note = Synthetic Construct
  source 1..30: mol_type = other RNA
ggcggaggaggcagcggcggaggaggcagc

SEQ ID NO: 53 - RNA, 741 nt, synthetic construct
  misc_feature 1..741: note = Synthetic Construct
  source 1..741: mol_type = other RNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 54 - protein, 419 aa, Homo sapiens
MSFIPVAEDSDFPIHNLPYGVFSTRGDPRPRIGVAIGDQILDLSIIKHLFTGPVLSKHQDVFNQPTLNSFMGLGQAAWKEARVFLQNLLSVSQARLRDDTELRKCAFISQASATMHLPATIGDYTDFYSSRQHATNVGIMFRDKENALMPNWLHLPVGYHGRASSVVVSGTPIRRPMGQMKPDDSKPPVYGACKLLDMELEMAFFVGPGNRLGEPIPISKAHEHIFGMVLMNDWSARDIQKWEYVPLGPFLGKSFGTTVSPWVVPMDALMPFAVPNPKQDPRPLPYLCHDEPYTFDINLSVNLKGEGMSQAATICKSNFKYMYWTMLQQLTHHSVNGCNLRPGDLLASGTISGPEPENFGSMLELSWKGTKPIDLGNGQTRKFLLDGDEVIITGYCQGDGYRIGFGQCAGKVLPALLPS

SEQ ID NO: 55 - protein, 354 aa, Homo sapiens
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGLALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 56 - protein, 1163 aa, Homo sapiens [sequence omitted]

SEQ ID NO: 57 - protein, 1038 aa, Homo sapiens
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 58 - protein, 1195 aa, Homo sapiens
MPTAESEAKVKTKVRFEELLKTHSDLMREKKKLKKKLVRSEENISPDTIRSNLHYMKETTSDDPDTIRSNLPHIKETTSDDVSAANTNNLKKSTRVTKNKLRNTQLATENPNGDASVEEDKQGKPNKKVIKTVPQLTTQDLKPETPENKVDSTHQKTHTKPQPGVDHQKSEKANEGREETDLEEDEELMQAYQCHVTEEMAKEIKRKIRKKLKEQLTYFPSDTLFHDDKLSSEKRKKKKEVPVFSKAETSTLTISGDTVEGEQKKESSVRSVSSDSHQDDEISSMEQSTEDSMQDDTKPKPKKTKKKTKAVADNNEDVDGDGVHEITSRDSPVYPKCLLDDDLVLGVYIHRTDRLKSDFMISHPMVKIHVVDEHTGQYVKKDDSGRPVSSYYEKENVDYILPIMTQPYDFKQLKSRLPEWEEQIVFNENFPYLLRGSDESPKVILFFEILDFLSVDEIKNNSEVQNQECGFRKIAWAFLKLLGANGNANINSKLRLQLYYPPTKPRSPLSVVEAFEWWSKCPRNHYPSTLYVVRGLKVPDCIKPSYRSMMAPQEEKGKPVHCERHHESSSVDTEPGLEESKEVIKWKRLPGQACRIPNKHLFSLNAGERGCFCLDFSHNGRILAAACASRDGYPIILYEIPSGRFMRELCGHLNIIYDLSWSKDDHYILTSSSDGTARIWKNEINNTNTFRVLPHPSFVYTAKFHPAVRELVVTGCYDSMIRIWKVEMREDSAILVRQFDVHKSFINSLCFDTEGHHMYSGDCTGVIVVWNTYVKINDLEHSVHHWTINKEIKETEFKGIPISYLEIHPNGKRLLIHTKDSTLRIMDLRILVARKFVGAANYREKIHSTLTPCGTFLFAGSEDGIVYVWNPETGEQVAMYSDLPFKSPIRDISYHPFENMVAFCAFGQNEPILLYIYDFHVAQQEAEMFKRYNGTFPLPGIHQSQDALCTCPKLPHQGSFQIDEFVHTESSSTKMQLVKQRLETVTEVIRSCAAKVNKNLSFTSPPAVSSQQSKLKQSNMLTAQEILHQFGFTQTGIISIERKPCNHQVDTAPTVVALYDYTANRSDELTIHRGDIIRVFFKDNEDWWYGSIGKGQEGYFPANHVASETLYQELPPEIKERSPPLSPEEKTKIEKSPAPQKQSINKNKSQDFRLGSESMTHSEMRKEQSHEDQGHIMDTRMRKNKQAGRKVTLIE

SEQ ID NO: 59 - protein, 558 aa, Homo sapiens
MAQDSVDLSCDYQFWMQKLSVWDQASTLETQQDTCLHVAQFQEFLRKMYEALKEMDSNTVIERFPTIGQLLAKACWNPFILAYDESQKILIWCLCCLINKEPQNSGQSKLNSWIQGVLSHILSALRFDKEVALFTQGLGYAPIDYYPGLLKNMVLSLASELRENHLNGFNTQRRMAPERVASLSRVCVPLITLTDVDPLVEALLICHGREPQEILQPEFFEAVNEAILLKKISLPMSAVVCLWLRHLPSLEKAMLHLFEKLISSERNCLRRIECFIKDSSLPQAACHPAIFRVVDEMFRCALLETDGALEIIATIQVFTQCFVEALEKASKQLRFALKTYFPYTSPSLAMVLLQDPQDIPRGHWLQTLKHISELLREAVEDQTHGSCGGPFESWFLFIHFGGWAEMVAEQLLMSAAEPPTALLWLLAFYYGPRDGRQQRAQTMVQVKAVLGHLLAMSRSSSLSAQDLQTVAGQGTDTDLRAPAQQLIRHLLLNFLLWAPGGHTIAWDVITLMAHTAEITHEIIGFLDQTLYRWNRLGIESPRSEKLARELLKELRTQV

SEQ ID NO: 60 - protein, 1274 aa, Homo sapiens [sequence omitted]

SEQ ID NO: 61 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 62 - protein, 1272 aa, synthetic construct
  REGION 1..1272: note = Synthetic Construct
FVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 63 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 64 - DNA, 669 bp, SARS-CoV-2
  source 1..669: mol_type = unassigned DNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaacatcgcagattacaactacaaattgcctgatgatttcactggttgcgttatcgcgtggaactctaataacctcgattctaaggtcggtggtaactacaattacctgtaccgcttgtttaggaagtcaaacctgaagcctttcgagagggatatttcaaccgaaatctatcaagcgggttcaacaccgtgtaacggtgtgaaaggatttaactgctacttccccctgcagtcttacggattccagccaacctatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 65 - protein, 18 aa, SARS-CoV-2
GIYQTSNFRVQPTESIVR

SEQ ID NO: 66 - protein, 17 aa, SARS-CoV-2
RVQPTESIVRFPNITNL

SEQ ID NO: 67 - protein, 17 aa, SARS-CoV-2
IVRFPNITNLCPFGEVF

SEQ ID NO: 68 - protein, 18 aa, SARS-CoV-2
TNLCPFGEVFNATRFASV

SEQ ID NO: 69 - protein, 18 aa, SARS-CoV-2
VFNATRFASVYAWNRKRI

SEQ ID NO: 70 - protein, 17 aa, SARS-CoV-2
SVYAWNRKRISNCVADY

SEQ ID NO: 71 - protein, 17 aa, SARS-CoV-2
KRISNCVADYSVLYNSA

SEQ ID NO: 72 - protein, 18 aa, SARS-CoV-2
ADYSVLYNSASFSTFKCY

SEQ ID NO: 73 - protein, 17 aa, SARS-CoV-2
SASFSTFKCYGVSPTKL

SEQ ID NO: 74 - protein, 18 aa, SARS-CoV-2
KCYGVSPTKLNDLCFTNV

SEQ ID NO: 75 - protein, 18 aa, SARS-CoV-2
KLNDLCFTNVYADSFVIR

SEQ ID NO: 76 - protein, 18 aa, SARS-CoV-2
NVYADSFVIRGDEVRQIA

SEQ ID NO: 77 - protein, 18 aa, SARS-CoV-2
IRGDEVRQIAPGQTGKIA

SEQ ID NO: 78 - protein, 16 aa, SARS-CoV-2
IAPGQTGKIADYNYKL

SEQ ID NO: 79 - protein, 18 aa, SARS-CoV-2
GKIADYNYKLPDDFTGCV

SEQ ID NO: 80 - protein, 18 aa, SARS-CoV-2
KLPDDFTGCVIAWNSNNL

SEQ ID NO: 81 - protein, 18 aa, SARS-CoV-2
CVIAWNSNNLDSKVGGNY

SEQ ID NO: 82 - protein, 18 aa, SARS-CoV-2
NLDSKVGGNYNYLYRLFR

SEQ ID NO: 83 - protein, 17 aa, SARS-CoV-2
NYNYLYRLFRKSNLKPF

SEQ ID NO: 84 - protein, 18 aa, SARS-CoV-2
LFRKSNLKPFERDISTEI

SEQ ID NO: 85 - protein, 13 aa, SARS-CoV-2
PFERDISTEIYQA

SEQ ID NO: 86 - protein, 18 aa, SARS-CoV-2
RDISTEIYQAGSTPCNGV

SEQ ID NO: 87 - protein, 18 aa, SARS-CoV-2
YQAGSTPCNGVEGFNCYF

SEQ ID NO: 88 - protein, 17 aa, SARS-CoV-2
NGVEGFNCYFPLQSYGF

SEQ ID NO: 89 - protein, 18 aa, SARS-CoV-2
CYFPLQSYGFQPTNGVGY

SEQ ID NO: 90 - protein, 18 aa, SARS-CoV-2
GFQPTNGVGYQPYRVVVL

SEQ ID NO: 91 - protein, 17 aa, SARS-CoV-2
GYQPYRVVVLSFELLHA

SEQ ID NO: 92 - protein, 18 aa, SARS-CoV-2
VVLSFELLHAPATVCGPK

SEQ ID NO: 93 - protein, 17 aa, SARS-CoV-2
HAPATVCGPKKSTNLVK

SEQ ID NO: 94 - protein, 18 aa, SARS-CoV-2
GPKKSTNLVKNKCVNFNF

SEQ ID NO: 95 - protein, 18 aa, SARS-CoV-2
VKNKCVNFNFNGLTGTGV

SEQ ID NO: 96 - protein, 223 aa, synthetic construct
  REGION 1..223: note = Synthetic Construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 97 - protein, 223 aa, synthetic construct
  REGION 1..223: note = Synthetic Construct
RVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 98 - DNA, 669 bp, synthetic construct
  misc_feature 1..669: note = Synthetic Construct
  source 1..669: mol_type = other DNA
cgcgtccagccaaccgagagcatcgtcagatttcccaacattacaaatctgtgtcccttcggcgaggtgttcaacgccacacgcttcgcttcagtgtacgcatggaaccgcaagcgcatatctaactgcgtcgcggattattctgtcctctacaactccgcctctttctccaccttcaagtgctacggagtgtcaccgactaagctgaacgatctctgctttaccaacgtctacgcggactccttcgtgataagaggtgatgaagtgagacaaatagccccaggtcagactggtaagatcgcagattacaactacaaattgcctgatgatttcactggttgcgttatcgcgtggaactctaataacctcgattctaaggtcggtggtaactacaattacaggtaccgcttgtttaggaagtcaaacctgaagcctttcgagagggatatttcaaccgaaatctatcaagcgggttcaaagccgtgtaacggtgtggaaggatttaactgctacttccccctgcagtcttacggattccagccaaccaatggcgtgggttaccaaccttatcgcgtggtggttctgagtttcgaactgttgcacgctcccgccacggtatgcggtcccaagaagagcactaacttggtgaagaataagtgcgtgaatttc

SEQ ID NO: 99 - DNA, 669 bp, synthetic construct
  misc_feature 1..669: note = Synthetic Construct
  source 1..669: mol_type = other DNA
cgcgtccagccaaccgagagcatcgtcagatttcccaacattacaaatctgtgtcccttcgacgaggtgttcaacgccacacgcttcgcttcagtgtacgcatggaaccgcaagcgcatatctaactgcgtcgcggattattctgtcctctacaacctcgcccctttcttcaccttcaagtgctacggagtgtcaccgactaagctgaacgatctctgctttaccaacgtctacgcggactccttcgtgataagaggtgatgaagtgagacaaatagccccaggtcagactggtaacatcgcagattacaactacaaattgcctgatgatttcactggttgcgttatcgcgtggaactctaataagctcgattctaaggtcagtggtaactacaattacctgtaccgcttgtttaggaagtcaaacctgaagcctttcgagagggatatttcaaccgaaatctatcaagcgggtaacaagccgtgtaacggtgtggcaggatttaactgctacttccccctgcggtcttacagcttccggccaacctatggcgtgggtcaccaaccttatcgcgtggtggttctgagtttcgaactgttgcacgctcccgccacggtatgcggtcccaagaagagcactaacttggtgaagaataagtgcgtgaatttc

(19) United States
(12) Patent Application Publication
(10) Pub. No.: US 2025/0108134 A1
(43) Pub. Date: Apr. 3, 2025

(54) CIRCULAR RNA VACCINES AND METHODS OF USE THEREOF

(71) Applicants: Beijing Changping Laboratory, Beijing (CN); Peking University, Beijing (CN); Therorna Inc., Beijing (CN)

(72) Inventors: Wensheng WEI, Beijing (CN); Yong SHEN, Beijing (CN); Liang QU, Beijing (CN); Feng CHEN, Beijing (CN); Zongyi YI, Beijing (CN)

(21) Appl. No.: 18/832,465

(22) PCT Filed: Jan. 28, 2023
(86) PCT No.: PCT/CN2023/073619

§ 371 Date: Jul. 23, 2024

(30) Foreign Application Priority Data
Jan. 28, 2022 (WO) PCT/CN2022/074738

(51) Int. Cl. A61K 48/00; A61K 38/17; A61K 38/39; A61K 38/45; A61K 38/46; A61K 38/47; A61K 39/00; A61K 39/215; A61P 37/04

(52) U.S. Cl. CPC A61K 48/0066; A61K 38/1709; A61K 38/1719; A61K 38/179; A61K 38/1793; A61K 38/39; A61K 38/45; A61K 38/46; A61K 38/47; A61K 39/0011; A61K 39/215; A61P 37/04; A61K 2039/53; A61K 2039/54

(57) ABSTRACT

Provided are methods of treating a disease or condition by administering a circular RNA (circRNA) encoding a therapeutic polypeptide (e.g., an antigenic polypeptide, a functional protein, a receptor protein, or a targeting protein (e.g., antibody)), wherein the circRNA is naked; and pharmaceutical composition(s) comprising the circRNA(s) as disclosed herein.

### CROSS-REFERENCE TO RELATED APPLICATIONS

This application claims priority to International Patent Application No. PCT/CN2022/074738 filed Jan. 28, 2022, the disclosure of which is hereby incorporated by reference in its entirety.

### REFERENCE TO AN ELECTRONIC SEQUENCE LISTING

The content of the electronic sequence listing (165392001141SEQLIST.xml; Size: 144,976 bytes; and Date of Creation: Jan. 23, 2023) is incorporated herein by reference in its entirety.

### FIELD

The present application relates to circular RNA (circRNA) encoding a therapeutic polypeptide, such as circRNA vaccines against a coronavirus, and methods of use thereof.

### BACKGROUND

Coronavirus disease 2019 (COVID-19) is a serious worldwide public health emergency caused by a coronavirus infection with the severe acute respiratory syndrome coronavirus 2 (SARS-COV-2). Currently, no effective drugs or vaccines are available to completely prevent or treat SARS-COV-2 variants. Thus, there is an urgent need for the development of safe and effective vaccines for coronavirus infections, such as SARS-COV-2. Vaccines typically fall into two broad categories: vaccines comprising a complete virus (live attenuated vaccines or inactivated vaccines), or vaccines comprising a part of a virus, which can be recombinant protein or DNA or RNA-based vaccines. Vaccines based on a complete virus are subject to several disadvantages, including the need to handle large amounts of an infectious virus during vaccine production for an inactivated vaccine, and the need for extensive safety testing of live attenuated vaccines. Vaccines based on recombinant protein are also limited by global production capacity of recombinant proteins, while DNA-based vaccines suffer from difficulties related to safe delivery of DNA and effectiveness to generate immune responses (Amanat, F. & Krammer, F. SARS-COV-2 Vaccines: Status Report. (2020) Immunity 52, 583-589).

The development of RNA-based vaccines provides a potential pathway to an immunogenic vaccine without requiring handling of infectious virus during production. RNA molecules are considered to be significantly safer than DNA vaccines, as RNAs are more easily degraded. They are cleared quickly out of the organism and cannot integrate into the genome and influence the cell's gene expression in an uncontrollable manner. It is also less likely for RNA vaccines to cause severe side effects like the generation of autoimmune disease or anti-DNA antibodies (Bringmann A. et al., Journal of Biomedicine and Biotechnology (2010), vol. 2010, article ID623687, incorporated herein by reference in its entirety). Transfection with RNA requires only insertion into the cell's cytoplasm, which is easier to achieve than into the nucleus.

CircRNAs and uses therefor have been described in PCT/CN2021/113865 and PCT/CN2021/115029, each of which is incorporated herein by reference in its entirety. All references described herein are hereby incorporated by reference in their entireties.

### BRIEF SUMMARY

The present application in one aspect provides a method of treating or preventing an disease or condition in an individual, comprising administering to the individual an effective amount of a circular RNA (circRNA) comprising a nucleic acid sequence encoding a therapeutic polypeptide, wherein the circRNA is a naked circRNA.

In some embodiments according to any of the methods described above, the disease or condition is an infection. In some embodiments, the infection is a coronavirus infection. In some embodiments, the infection is SARS-COV-2 infection, optionally the SARS-COV-2 infection is caused by a SARS-COV-2 variant (e.g., a delta variant or an omicron variant or sub-variants thereof). In some embodiments, the SARS-COV-2 infection is caused by one or more SARS-COV-2 variants. In some embodiments, the SARS-COV-2 infection is caused by one or more omicron sub-variants (e.g. BA.1, BA.1.1, BA.2, BA.3, BA.4, or BA.5). In some embodiments, the SARS-CoV-2 infection is caused by one or more sublineages of omicron sub-variants (e.g. BA.2.75.2, BA.4.6, BF.7, or BQ.1.1). In some embodiments, the therapeutic polypeptide is selected from the group consisting of an antigenic polypeptide, a functional protein, a receptor protein, and a targeting protein. In some embodiments, the therapeutic polypeptide is an antigenic polypeptide, for example, an antigenic polypeptide present in a vaccine.

In some embodiments according to any of the methods described above, the disease or condition is a disease or condition associated with insufficient levels and/or activity of a protein corresponding to the therapeutic polypeptide, or wherein the disease or condition is a hereditary genetic disease associated with one or more mutations in the protein corresponding to the therapeutic polypeptide, wherein the therapeutic polypeptide is selected from the group consisting of an antigenic polypeptide, a functional protein, a receptor protein, and a targeting protein. In some embodiments, the (i) the therapeutic polypeptide is TP53 or PTEN, and the disease or condition is cancer; (ii) the therapeutic polypeptide is OTC, and the disease or condition is ornithine transcarbamylase deficiency; (iii) the therapeutic polypeptide is FAH, and the disease or condition is tyrosinemia; (iv) the therapeutic polypeptide is DMD, and the disease or condition is Duchenne and Becker muscular dystrophy, X-linked dilated cardiomyopathy, or familial dilated cardiomyopathy; (v) the therapeutic polypeptide is IDUA, and the disease or condition is Mucopolysaccharidosis type I (MPS I); (vi) the therapeutic polypeptide is COL3A1, and the disease or condition is Ehlers-Danlos syndrome; (vii) the therapeutic polypeptide is AHI1, and the disease or condition is Joubert syndrome; (viii) the therapeutic polypeptide is BMPR2, and the disease or condition is pulmonary arterial hypertension, or pulmonary veno-occlusive disease; (ix) the therapeutic polypeptide is FANCC, and the disease or condition is Fanconi anemia; (x) the therapeutic polypeptide is MYBPC3, and the disease or condition is primary familial hypertrophic cardiomyopathy; or (xi) the therapeutic polypeptide is IL2RG, and the disease or condition is X-linked severe combined immunodeficiency.

In some embodiments according to any of the methods described above, the circRNA is subject to rolling circle translation by a ribosome in the individual.

In some embodiments according to any of the methods described above, the circRNA is administered two or more times to the individual. In some embodiments, the interval between each administration is at least about four weeks, such as at least any of five, six, seven, or eight weeks.

In some embodiments according to any of the methods described above, the circRNA is formulated as a solution. In some embodiments, the concentration of the circRNA in the solution is about 1 μg/mL to about 10,000 μg/mL, including for example about 10 μg/mL to about 1,000 μg/mL, about 100 μg/mL to about 500 μg/mL, about 200 μg/mL to about 300 μg/mL. In some embodiments, the solution is substantially free of an adjuvant. In some embodiments, the solution comprises an adjuvant, including for example an adjuvant which is not aluminum hydroxide, optionally wherein the solution is substantially free of aluminum hydroxide.

In some embodiments according to any of the methods described above, the circRNA is administered intravenously, intramuscularly, subcutaneously, transdermally, or via lymph node. In some embodiments, the circRNA is administered at a dose of about 1 μg to about 10,000 μg, including for example about 10 μg to about 1,000 μg, about 100 μg to about 500, about 200 to about 300 μg.

In some embodiments according to any of the methods described above, the circRNA further comprises a Kozak sequence operably linked to the nucleic acid sequence encoding a therapeutic polypeptide.

In some embodiments according to any one of the methods described above, the circRNA further comprises an in-frame 2A peptide coding sequence operably linked to the 3′ end of the nucleic acid sequence encoding a therapeutic polypeptide.

In some embodiments according to any one of the methods described above, the circRNA further comprises an internal ribosomal entry site (IRES) sequence operably linked to the nucleic acid sequence encoding a therapeutic polypeptide. In some embodiments, the IRES sequence is a CVB3 virus, EV71 virus, EMCV virus, PV virus, or CSFV virus IRES sequence. In some embodiments, the circRNA comprises a nucleic acid sequence comprising from the 5′ end to the 3′ end: the IRES sequence, the Kozak sequence, and the nucleic acid sequence encoding a therapeutic polypeptide. In some embodiments according to any one of the methods involving circRNAs comprising an IRES sequence described above, the circRNA further comprises a polyAC or polyA sequence disposed at the 5′ end of the IRES.

In some embodiments according to any one of the methods described above, the circRNA further comprises an m6A modification motif sequence operably linked to the nucleic acid sequence encoding a therapeutic polypeptide. In some embodiments, the circRNA comprises a nucleic acid sequence comprising from the 5′ to the 3′ end: the m6A modification motif sequence, the Kozak sequence, and the nucleic acid sequence encoding a therapeutic polypeptide.

In some embodiments according to any one of the methods described above, the nucleic acid further encodes a signal peptide (SP) fused to the N-terminus of a therapeutic polypeptide. In some embodiments, the SP is an SP of a human tissue plasminogen activator (tPA) or an SP of a human IgE immunoglobulin (e.g., the sequence shown in SEQ ID NO: 16). In some embodiments, the SP is an SP of a human IgE immunoglobulin, (e.g., the sequence shown in SEQ ID NO: 17).

In some embodiments according to any one of the methods described above, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence comprises the nucleic acid sequence of SEQ ID NO: 21, and the 5′ exon sequence comprises the nucleic acid sequence of SEQ ID NO: 22.

In some embodiments according to any one of the methods described above for treating or preventing an infection, the therapeutic polypeptide is an antigenic peptide. In some embodiments, the infection is an infection by coronavirus. In some embodiments, the coronavirus is selected from the group consisting of SARS-COV, MERS-COV, and SARS-COV-2. In some embodiments, the coronavirus is SARS-COV-2 (e.g., of a delta strain or omicron strain or sub-variants thereof). In some embodiments, the SARS-COV-2 infection is caused by one or more SARS-COV-2 variants. In some embodiments, the SARS-COV-2 infection is caused by one or more omicron sub-variants (e.g. BA.1, BA.1.1, BA.2, BA.3, BA.4, or BA.5). In some embodiments, the SARS-COV-2 infection is caused by one or more sublineages of omicron sub-variants (e.g. BA.2.75.2, BA.4.6, BF.7, or BQ.1.1).

In some embodiments, the antigenic polypeptide comprises a Spike(S) protein or a fragment thereof of a coronavirus. In some embodiments, the S protein or fragment thereof comprises a D614G mutation. In some embodiments, the antigenic polypeptide comprises an amino acid sequence having at least about 80% (e.g., at least about 85%, 90%, 95%, 98%, or more, or 100%) sequence identity to an amino acid sequence selected from the group consisting of SEQ ID NOs: 8-10, 62, 63, and 65-97. In some embodiments, the circRNA comprises a nucleic acid sequence having at least about 80% (e.g., at least about 85%, 90%, 95%, 98%, or more, or 100%) sequence identity to a nucleic acid sequence selected from the group consisting of SEQ ID NOs: 11-15, 64, 98, and 99.

In some embodiments, the antigenic polypeptide comprises a receptor-binding domain (RBD) of the S protein, optionally wherein the RBD comprises amino acid residues 319 to 542 of a full-length S protein of SARS-COV-2, wherein the numbering is based on SEQ ID NO: 1.

In some embodiments according to any one of the method described above involving circRNAs encoding an antigenic polypeptide described above, the antigenic polypeptide comprises a receptor-binding domain (RBD) of the S protein. In some embodiments, the RBD comprises amino acid residues 319 to 542 of a full-length S protein of SARS-COV-2, wherein the numbering is based on SEQ ID NO: 1. In some embodiments, the RBD comprises an amino acid sequence having at least about 80% identity (e.g., at least about 85%, 90%, 95%, 98%, or more, or 100% identity) to the amino acid sequence of SEQ ID NO: 2. In some embodiments, the antigenic polypeptide comprises a receptor-binding domain (RBD) of the S protein, wherein the RBD comprises an amino acid sequence having at least about 80% identity (e.g., at least about 85%, 90%, 95%, 98%, 99% or more, or 100% identity) to the amino acid sequence of SEQ ID NO: 63.

In some embodiments according to any one of the methods described above involving circRNAs comprising an RBD described above, the antigenic polypeptide further comprises a multimerization domain. In some embodiments, the multimerization domain is a C-terminal Foldon (Fd) domain of a T4 fibritin protein or a GCN4-based isoleucine zipper domain. In some embodiments, the multimerization domain comprises an amino acid sequence having at least about 80% (e.g., at least about 85%, 90%, 95%, 98%, or more, or 100%) to the amino acid sequence of SEQ ID NO: 3 or SEQ ID NO: 4. In some embodiments, the RBD is fused to the multimerization domain via a peptide linker. In some embodiments, the peptide linker comprises the amino acid sequence of SEQ ID NO: 5.

In some embodiments according to any one of the methods described above involving circRNAs encoding an antigenic polypeptide described above, the antigenic polypeptide comprises an S2 region of the S protein. In some embodiments, the S2 region comprises amino acid residues 686 to 1273 of a full-length S protein of SARS-COV-2, wherein the numbering is based on SEQ ID NO: 1. In some embodiments, the S2 region comprises one or more mutations that stabilize a pre-fusion conformation of the S protein. In some embodiments, the one or more mutations comprise K986P and V987P. In some embodiments, the S2 region comprises an amino acid sequence having at least about 80% (e.g., at least about 85%, 90%, 95%, 98%, or more, or 100%) to the amino acid sequence of SEQ ID NO: 6 or 7.

In some embodiments according to any one of the methods described above involving circRNAs encoding an antigenic polypeptide described above, the antigenic polypeptide comprises amino acid residues 2-1273 of a full-length S protein of SARS-COV-2, wherein the numbering is based on SEQ ID NO: 1. In some embodiments, the antigenic polypeptide comprises one or more mutations that inhibit cleavage of the S protein. In some embodiments, the one or more mutations that inhibit cleavage of the S protein comprise deletion of amino acid residues 681-684, wherein the numbering is based on SEQ ID NO: 1. In some embodiments, the SARS-CoV-2 is a Delta variant, and wherein the antigenic polypeptide comprises the amino acid sequence of SEQ ID NO: 96. In some embodiments, the SARS-COV-2 is an Omicron variant, and wherein the antigenic polypeptide comprises the amino acid sequence of SEQ ID NO: 97. In some embodiments, the SARS-COV-2 is one or more sub-variants of an Omicron variant (e.g. BA.1, BA.1.1, BA.2, BA.3, BA.4, or BA.5). In some embodiments, the SARS-COV-2 is one or more sublineages of omicron sub-variants (e.g. BA.2.75.2, BA.4.6, BF.7, or BQ.1.1).

In some embodiments according to any one of the methods described above, the therapeutic polypeptide is a receptor protein, optionally a soluble receptor protein comprising an extracellular domain of a naturally occurring receptor. In some embodiments, the receptor protein is an ACE-2 receptor, including a high-affinity mutant ACE-2 receptor. In some embodiments, the therapeutic polypeptide is a targeting protein, including an antibody (such as a neutralizing antibody, or a therapeutic antibody). In some embodiments, the therapeutic polypeptide is a functional protein, for example, an enzyme (such as OTC, FAH, and IDUA), p53, and PTEN. In some embodiments, the functional protein is selected from the group consisting of DMD, COL3A1, BMPR2, AHI1, FANCC, MYBPC3, and IL2RG.

In some embodiments, the method comprises administering to the individual a plurality of circRNAs described herein, wherein the therapeutic polypeptides corresponding to the plurality of circRNAs are different with respect to each other.

Also provided are compositions, kits and articles of manufacture for use in any one the methods described above. For example, in some embodiments, there is provided a pharmaceutical composition (such as a vaccine composition) comprising the circRNAs described in any of the embodiments above, wherein the pharmaceutical composition is suitable for administration of naked circRNA, for example when not formulated with a transfection agent.

### BRIEF DESCRIPTION OF THE DRAWINGS

FIG. 1A shows an exemplary method of generating a circRNA vaccine in vitro based on a Group I catalytic intron. A typical Group I catalytic intron comprises, from the 5′ end to the 3′ end: a 5′ exon comprising a 5′ exon sequence recognizable by a 5′ catalytic Group I intron fragment (Exon 1), 5′ catalytic Group I intron fragment, 3′ catalytic Group I intron fragment, and a 3′ exon comprising a 3′ exon sequence recognizable by the 3′ catalytic Group I intron fragment (Exon 2). A linear RNA construct with an insert sequence can be made to allow auto-catalysis of the Group I intron fragments in order to join the two ends of the insert sequence and obtain a circular RNA after self-splicing by the Group I intron. The linear construct comprises, from 5′ to 3′, 3′ catalytic Group I intron fragment, a 3′ exon (Exon 2), an insert sequence, a 5′ exon (Exon 1), and 5′ Group I intron. The insert sequence may comprise the nucleic acid sequence encoding the antigenic polypeptide. FIG. 1B shows a schematic of an exemplary nucleotide sequence having an IRES, and an exemplary method of circularizing purified linear RNAs by ribozyme autocatalysis of the Group I catalytic Intron. FIG. 1C shows a schematic of an exemplary nucleotide sequence having an m6A modification motif sequence before the start codon, and an exemplary method of circularizing purified linearized RNAs by ribozyme autocatalysis of the Group I catalytic intron.

FIG. 2A shows a schematic of an exemplary nucleotide sequence having an IRES, and an exemplary method of circularizing linear RNAs by enzyme catalysis using T4 RNA ligases by supplying ssDNA adaptors. FIG. 2B shows a schematic of an exemplary nucleotide sequence wherein the IRES sequence is replaced with an m6A modification motif and the TAA stop codon is replaced with a 2A peptide coding sequence (in non-limiting examples, a T2A, P2A or other 2A peptide-coding sequence). Also shown is an exemplary method of circularizing linear RNAs by enzyme catalysis using T4 RNA ligases by supplying ssDNA adaptors. FIG. 2C shows a schematic illustration of the ribosome rolling circle translation of the circRNA vaccine. The translation factors can be recruited and translation initiated by either the IRES site or the m6A modification motifs.

FIG. 3A shows the results of agarose gel electrophoresis of an exemplary purified circRNA^{RBD} and precursor RNA (LinRNA^{RBD}, wherein the 3′ Intron sequence was mutated to random sequence), demonstrating that the circRNA^{RBD} ran faster than LinRNA^{RBD}, indicating circularization of the RNA. FIG. 3B shows the results of an endonuclease RNase R digestion assay of an exemplary circRNA (circRNA^{RBD}) or LinRNA (LinRNA^{RBD}). Following incubation with RNAse R for the indicated time periods, the reaction products were resolved in agarose gel electrophoresis, indicating that the circRNA, which lacks a 5′ or 3′ end, was more resistant to RNase R compared to the LinRNA. FIG. 3C shows the agarose gel electrophoresis result of the PCR products of linear RNA^{RBD} and circRNA^{RBD}, using the primers shown in FIG. 3E. FIG. 3D shows the results of a quantitative ELISA assay to measure the concentration of RBD antigens in the supernatant. The data were shown as the mean±S.E.M. (n=3). FIG. 3E shows a schematic diagram of circRNA^{RBD} circularization by the Group I ribozyme autocatalysis. SP, signal peptide sequence of human tPA protein. T4, the trimerization domain from bacteriophage T4 fibritin protein. RBD, the receptor binding domain of SARS-COV-2 Spike protein. The arrows indicate the the design of primers for PCR analysis shown in FIG. 3C.

FIGS. 4A-4B show Western Blot analysis demonstrating expression and secretion of an exemplary protein from eukaryotic cells after transfection with an exemplary circRNA. Human HEK293T cells (FIG. 4A) and mouse NIH3T3 (FIG. 4B) cells were transfected with circRNA^{RBD} or circRNA^{EGFP} or the precursor RNA named LinRNA^{RBD} as controls. After 48 hours, the culture supernatant of transfected cells was collected for Western Blot analysis. Using the SARS-COV-2 Spike RBD antibody (ABclonal, A20135) for detection, the Western Blot results showed that the circRNA^{RBD} could express and secret the SARS-COV-2 RBD antigen to the cellular supernatant efficiently. FIG. 4C shows results demonstrating the stability of an exemplary circRNA after extended incubation at room temperature. Purified circRNA^{RBD} was kept at room temperature about 25° C. for 3, 7 or 14 days, and then transfected into human HEK293T cells. The Western Blot results showed that the SARS-COV-2 RBD antigen could be expressed by the circRNA^{RBD} and secreted to the cellular supernatant efficiently, even when the circRNA^{RBD} was kept at room temperature for 14 days.

FIGS. 5A-5B show results of a pseudovirus competition experiment demonstrating that the secreted SARS-COV-2 RBD antigen produced by circRNA effectively interfered with infection of cells by a SARS-COV-2 pseudovirus. Collected supernatant from HEK293T cells transfected with circRNA^{RBD} or controls was incubated with a lentivirus-based SARS-COV-2 pseudovirus expressing an EGFP fluorescence marker at 37° C. for 2 hours. The resulting supernatant was then added into the culture medium of ACE2-overexpressing cells named HEK293-ACE2. After 48 hours, the cells were collected for FACS analysis for the EGFP marker, indicating infection of the cells by the pseudovirus. The results are shown as a bar graph in FIG. 5A, and the FACS plots are shown in FIG. 5B.

FIG. 6A shows an exemplary method of generating a circRNA and a schematic of an exemplary circRNA construct for expression of a neutralizing antibody, such as a SARS-COV-2 neutralizing antibody. Although the construct shown comprises an IRES sequence, it will be appreciated that any of the exemplary circRNA constructs described herein can be used for expression of a secreted neutralizing antibody (e.g., a variation of the construct comprising an m6A site and/or a 2A peptide instead of a stop codon, as shown in FIG. 1C). FIG. 6B shows the pseudovirus neutralization activity of a secreted nAb produced by exemplary circRNA-nAb constructs (circRNA^{nAb-1}, comprising a nucleotide sequence encoding nAb-1 (amino acid sequence shown in SEQ ID NO: 27), circRNA^{nAb-2}, comprising a nucleotide sequence encoding nAb-2 (amino acid sequence shown in SEQ ID NO: 28), and, and circRNA^{nAB-5}, comprising a nucleotide sequence encoding nAb-5 (amino acid sequence shown in SEQ ID NO: 31). A circRNA expressing Luciferase (circRNA^{Luc}) and a linear RNA encoding nAb-5 (LinRNA^{nAB-5}) were used as negative controls and a commercial SARS-COV-2 neutralizing antibody (ABclonal, A19215) was used as the positive control. FIG. 6C shows the results of lentivirial-based pseudovirus neutralization assay with the supernatant from cells transfected with circRNA encoding neutralizing nanobodies nAB1, nAB1-Tri, nAB2, nAB2-Tri, nAB3 and nAB3-Tri or ACE2 decoys. The luciferase value was normalized to the circRNA^{EGFP} control. The data were shown as the mean±S.E.M. (n=2). FIG. 6D shows the results of a neutralization assay of VSV-based D614G, B.1.1.7 or B.1.351 pseudovirus by the supernatant from cells transfected with neutralizing nanobodies nAB1-Tri, nAB3-Tri or ACE2 decoys expressed through the circRNA platform. The data were shown as the mean±S.E.M. (n=3).

FIG. 7 shows an exemplary method of generating a circRNA and a schematic of an exemplary circRNA construct for expression of a therapeutic polypeptide, such as IDUA. A mouse α-l-iduronidase (IDUA) coding sequence was inserted into the circRNA backbone. Although the construct shown comprises an IRES sequence, and a nucleotide sequence coding for IDUA, it will be appreciated that any of the exemplary circRNA constructs described herein can be used for expression of any of the therapeutic polypeptides described herein (e.g., a variation of the construct comprising an m6A site and/or a 2A peptide instead of a stop codon, as shown in FIG. 1C).

FIG. 8 shows the schematic diagram of circRNA^{RBD-501Y.V2} circularization by the Group I ribozyme autocatalysis. SP, signal peptide sequence of human tPA protein. T4, the trimerization domain from bacteriophage T4 fibritin protein. RBD-501Y.V2, the RBD antigen harboring the K417N-E484K-N501Y mutations in SARS-COV-2 501Y.V2 variant.

FIGS. 9A-9C show mice imaged by IVIS imaging system. FIG. 9A shows untreated mice. FIG. 9B shows mice injected with PBS. FIG. 9C shows mice injected with naked circRNA^{Luc}.

FIGS. 10A-10B show immunogenicity of naked SARS-COV-2 circRNA^{RBD-Beta} vaccines in mice. FIG. 10A shows schematic diagram of the circRNA^{RBD-Beta} vaccination process in BALB/c mice and serum collection schedule for specific antibodies analysis. FIG. 10B shows measurement of the SARS-COV-2 specific IgG antibody titer with ELISA. The data were shown as the geometric mean±geometric S.D.

FIGS. 11A-11B show immunogenicity of naked SARS-COV-2 circRNA^{RBD-Delta} vaccines in mice. FIG. 11A shows schematic diagram of the circRNA^{RBD-Delta} vaccination process in BALB/c mice and serum collection schedule for specific antibodies analysis. FIG. 11B shows measurement of the SARS-COV-2 specific IgG antibody titer with ELISA. The data were shown as the geometric mean±geometric S.D.

FIGS. 12A-12C show immunogenicity of naked RNA^{RBD-Delta} vaccines in mice. FIG. 12A shows a schematic diagram of the naked RNA RBD-Beta vaccines vaccination process in BALB/c mice and serum collection schedule for specific antibodies analysis. FIG. 12B shows results of comparing the SARS-COV-2 RBD-Delta specific antibodies titer induced by naked circRNA vaccine or naked mRNA vaccines. FIG. 12C shows results of comparing the SARS-COV-2^{RBD-Delta} specific antibodies titer induced by naked circRNA vaccine dissolved in PBS or Ringer's solution. The data were shown as the geometric mean±geometric S.D.

### DETAILED DESCRIPTION

The present application provides methods of treating or preventing a disease or condition (including infection) in an individual, comprising administering to the individual an effective amount of circRNAs encoding a therapeutic polypeptide, such as an antigenic polypeptide, a functional protein, a receptor protein, or a targeting protein (e.g., antibody), wherein the cirRNA is a naked circRNA.

The invention is based, at least in part, on the surprising finding that a circRNA encoding an antigenic polypeptide, namely, a circRNA encoding an RBD antigen of a SAR-Cov-2 virus variant, when injected in naked form, could induce a high level of RBD-specific neutralizing antibodies in vivo. A method comprising administration of naked circRNA would therefore be a useful option for treatment and prevention of a disease or condition avoiding any toxicity issues triggered by delivery vehicles traditionally used for RNA therapeutics, such as lipid nanoparticles (LNP).

Given their circular nature, circRNAs are particularly stable compared to many linear RNAs because they are resistant to exonucleolytic decay by the cellular exosome ribonuclease complex. The present application leverages the advantageous properties of circRNA, and provides novel dosing regimens for the administration of circRNA-based therapeutics and vaccines.

Thus, the present application in one aspect provides a method of treating or preventing an infection in an individual, comprising administering to the individual an effective amount of circRNAs encoding an antigenic polypeptide, wherein the circRNA is a naked circRNA. In another aspect, there is provided a method of treating or preventing a disease or condition in an individual, comprising administering to the individual an effective amount of circRNAs encoding a therapeutic polypeptide, wherein the circRNA is a naked circRNA. Also provided are kits, vaccines, and pharmaceutical compositions useful for such methods.

### I. Definitions

Terms are used herein as generally used in the art, unless otherwise defined as follows.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof.

“Naked circRNA” described herein refers to circRNA that is free of a delivery vehicle (e.g., liposomes, lipid nanoparticles, or colloidal parties).

The term “vaccine” is understood as being directed to an immunoactive pharmaceutical preparation. In certain embodiments, the vaccine induces adaptive immunity when administered to a host. The vaccine preparation may further contain a pharmaceutical carrier, which may be designed for the particular mode by which the vaccine is intended to be administered.

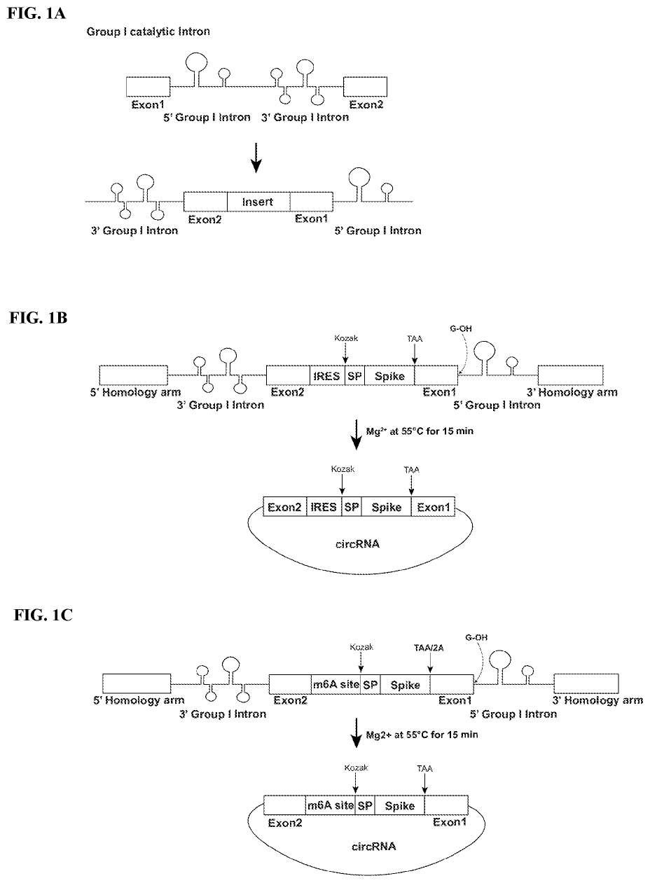

The terms “Group I intron” and “Group I catalytic intron” are used interchangeably to refer to a self-splicing ribozyme that can catalyze its own excision from an RNA precursor. Group I introns comprise two fragments, the 5′ catalytic Group I intron fragment and the 3′ catalytic Group I intron fragment, which retain their folding and catalytic function (i.e., self-splicing activity). In its native environment, the 5′ catalytic Group I intron fragment is flanked at its 5′ end by a 5′ exon, which comprises a 5′ exon sequence that is recognized by the 5′ catalytic Group I intron fragment; and the 3′ catalytic Group I intron fragment is flanked at its 3′ end by a 3′ exon, which comprises a 3′ exon sequence that is recognized by the 3′ catalytic Group I intron fragment. The terms “5′ exon sequence” and “3′ exon sequence” used herein are labeled according to the order of the exons with respect to the Group I intron in its natural environment, e.g., as shown in FIG. 1A.

The term “therapeutic polypeptide” refers to a polypeptide having a therapeutic effect. A therapeutic polypeptide may be a naturally-occurring protein or an engineered functional variant thereof, including functional fragments, derivatives having one or more mutations (e.g., insertion, deletion, substitution, etc.) to the amino acid sequence of the naturally-occurring protein, and fusion proteins comprising a naturally-occurring protein or fragment thereof. A therapeutic polypeptide may also be an engineered protein that does not have a naturally-occurring counterpart. Therapeutic polypeptide(s) may have a single polypeptide chain or multiple polypeptide chains.

The term “antigenic polypeptide” refers to a polypeptide that can be used to trigger the immune system of a mammal to develop antibodies specific to the polypeptide or a portion thereof. Antigenic polypeptides described herein include naturally-occurring proteins, protein domains, and short peptide fragments derived from a naturally-occurring protein. An antigenic polypeptide may contain one or more known epitopes of a naturally-occurring protein. The antigenic polypeptide may comprise a carrier protein or multimerization protein that improves immunogenicity.

As used herein, a “variant” virus refers to an isolate of a virus whose genome sequence differs from that of a reference virus and the difference in the genome sequence confers new phenotypic properties such as increased fitness compared to the reference virus. When referring to a viral species in the present application, such as SARS-COV-2, it is understood that the species encompass variants, sub-variants and sublineages as well as the reference virus that was first isolated and identified. In some embodiments, the variant virus is a variant being monitored (VBM) by the Centers for Disease Control and Prevention (“CDC”) of the United States, or by the World Health Organization (“WHO”). Variants designated as VBM include those where data indicates there is a potential or clear impact on approved or authorized medical countermeasures or that have been associated with more severe disease or increased transmission but are no longer detected, or are circulating at very low levels, in the United States. In some embodiments, the variant virus described herein is a “variant of interest”, i.e., a variant with specific genetic markers that have been associated with changes to receptor binding, reduced neutralization by antibodies generated against previous infection or vaccination, reduced efficacy of treatments, potential diagnostic impact, and/or predicted increase in transmissibility and/or disease severity. In some embodiments, the variant virus described herein is a “variant of concern”, i.e., a variant for which there is evidence of an increase in transmissibility, more severe disease (e.g., increased hospitalizations and/or deaths), significant reduction in neutralization by antibodies generated during previous infection or vaccination, reduced effectiveness of treatments or vaccines, and/or diagnostic detection failures. In some embodiments, the variant virus described herein is a “variant of high consequence”, i.e., a variant of high consequence has clear evidence that prevention measures or medical countermeasures (MCMs) have significantly reduced effectiveness relative to previously circulating variants.

The term “functional protein” refers to a naturally-occurring protein, functional variants thereof, or an engineered derivative thereof that is functional in treating a genetic disease or condition. The disease or condition may be caused in whole or in part by a change, such as a mutation, in the wildtype, naturally-occurring protein corresponding to the functional protein.

The term “targeting protein” refers to a polypeptide that specifically binds to a target molecule. Targeting proteins described herein include both antibody-based and non-antibody based binding proteins or target-binding portions thereof.

The term “antibody” is used in its broadest sense and encompasses various antibody structures, including but not limited to monoclonal antibodies, polyclonal antibodies, multispecific antibodies (e.g., bispecific antibodies), full-length antibodies and antigen-binding fragments thereof, so long as they exhibit the desired antigen-binding activity. The term “antigen-binding fragment” as used herein refers to an antibody fragment including, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain Fv (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure.

As use herein, the terms “specifically binds,” “specifically recognizing,” and “is specific for” refer to measurable and reproducible interactions, such as binding between a target and a targeting moiety. For example, a targeting moiety that specifically recognizes a target (which can be an epitope) is a targeting moiety (e.g., antibody) that binds this target with greater affinity, avidity, more readily, and/or with greater duration than its bindings to other molecules. In some embodiments, the extent of binding of a targeting moiety to an unrelated molecule is less than about 10% of the binding of the targeting moiety to the target as measured, e.g., by a radioimmunoassay (RIA). In some embodiments, a targeting moiety that specifically binds a target has a dissociation constant (KD) of ≤10⁻⁵ M, ≤10⁻⁶ M, ≤10⁻⁷ M, ≤10⁻⁸ M, ≤10⁻⁹ M, ≤10⁻¹⁰ M, ≤10⁻¹¹ M, or ≤10⁻¹² M. In some embodiments, specific binding can include, but does not require exclusive binding. Binding specificity of the targeting moiety can be determined experimentally by methods known in the art. Such methods comprise, but are not limited to Western blots, ELISA, RIA, ECL, IRMA, EIA, BIACORE™ and peptide scans.

The term “functional variant” of a reference protein refers to a variant polypeptide derived from the reference protein or a portion thereof, and the variant has substantially the same activity (e.g., binding to a target or enzymatic activity) as the reference protein. “Substantially the same activity” means an activity level that is at least about any one of 20%, 30%, 40%, 50%, 60%, 70%, 80%, 90%, or more as the activity of the reference protein.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein, “treatment” or “treating” is an approach for obtaining beneficial or desired results including clinical results. For purposes of this application, beneficial or desired clinical results include, but are not limited to, one or more of the following: decreasing one more symptoms resulting from the disease, diminishing the extent of the disease, stabilizing the disease (e.g., preventing or delaying the worsening of the disease), preventing or delaying the spread of the disease, preventing or delaying the occurrence or recurrence of the disease, delaying or slowing the progression of the disease, ameliorating the disease state, providing a remission (whether partial or total) of the disease, decreasing the dose of one or more other medications required to treat the disease, delaying the progression of the disease, increasing the quality of life, and/or prolonging survival. Also encompassed by “treatment” is a reduction of pathological consequence of the disease. The methods of the present application contemplate any one or more of these aspects of treatment.

The terms “individual,” “subject” and “patient” are used interchangeably herein to describe a mammal, including humans. In some embodiments, the individual is human. In some embodiments, the individual is a rodent, such as a mouse. In some embodiments, the individual suffers from a genetic disease or condition. In some embodiments, the individual suffers from a coronavirus infection. In some embodiments, the individual is at risk of contracting a coronavirus infection. In some embodiments, the individual is in need of treatment.

As is understood in the art, an “effective amount” refers to an amount of a composition sufficient to produce a desired therapeutic outcome (e.g., stimulating the production of antibodies and improving immunity against one or more coronaviruses, reducing the severity or duration of, stabilizing the severity of, or eliminating one or more symptoms of a coronavirus infection). For therapeutic use, beneficial or desired results include, e.g., decreasing one or more symptoms resulting from the disease (biochemical, histologic and/or behavioral), including its complications and intermediate pathological phenotypes presented during development of the disease, increasing the quality of life of those suffering from the disease, decreasing the dose of other medications required to treat the disease, enhancing effect of another medication, delaying the progression of the disease, and/or prolonging survival of patients. In some embodiments, an effective amount of the therapeutic agent may extend survival (including overall survival and progression free survival); result in an objective response (including a complete response or a partial response); relieve to some extent one or more signs or symptoms of the disease or condition; and/or improve the quality of life of the subject. In some embodiments, an effective amount is a prophylactically effective amount, which is an amount of a composition sufficient to prevent or reduce the severity of one or more future symptoms of a coronavirus infection when administered to an individual who is susceptible and/or who may develop the coronavirus infection. For prophylactic use, beneficial or desired results include, e.g., results such as eliminating or reducing the risk, lessening the severity of future disease, or delaying the onset of the disease (e.g., delaying biochemical, histologic and/or behavioral symptoms of the disease, its complications, and intermediate pathological phenotypes presenting during future development of the disease).

As used herein the term “wild type” is a term of the art understood by skilled persons and means the typical form of an organism, strain, gene or characteristic as it occurs in nature as distinguished from mutant or variant forms.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990), each of which is incorporated herein by reference in its entirety).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R.C., Nucleic Acids Research 32 (5): 1792-1797, 2004; Edgar, R.C., BMC Bioinformatics 5 (1): 113, 2004, each of which are incorporated herein by reference in their entirety for all purposes).

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The term “simultaneous administration,” as used herein, means that a first therapy and second therapy in a combination therapy are administered with a time separation of no more than about 15 minutes, such as no more than about any of 10, 5, or 1 minutes. When the first and second therapies are administered simultaneously, the first and second therapies may be contained in the same composition (e.g., a composition comprising both a first and second therapy) or in separate compositions (e.g., a first therapy in one composition and a second therapy is contained in another composition).

As used herein, the term “sequential administration” means that the first therapy and second therapy in a combination therapy are administered with a time separation of more than about 15 minutes, such as more than about any of 20, 30, 40, 50, 60, or more minutes. Either the first therapy or the second therapy may be administered first. The first and second therapies are contained in separate compositions, which may be contained in the same or different packages or kits.

As used herein, the term “concurrent administration” means that the administration of the first therapy and that of a second therapy in a combination therapy overlap with each other.

The term “pharmaceutical composition” refers to a preparation which is in such form as to permit the biological activity of an active ingredient contained therein to be effective, and which contains no additional components which are unacceptably toxic to a subject to which the formulation would be administered. In some embodiments, a pharmaceutical composition comprises any one of circRNAs described herein, and a pharmaceutically acceptable carrier.

A “pharmaceutically acceptable carrier” refers to one or more ingredients in a pharmaceutical formulation, other than an active ingredient, which is nontoxic to a subject. A pharmaceutically acceptable carrier includes, but is not limited to, a buffer, excipient, stabilizer, cryoprotectant, tonicity agent, preservative, and combinations thereof. Pharmaceutically acceptable carriers or excipients have preferably met the required standards of toxicological and manufacturing testing and/or are included on the Inactive Ingredient Guide prepared by the U.S. Food and Drug administration or other state/federal government or listed in the U.S. Pharmacopeia or other generally recognized pharmacopeia for use in mammals, and more particularly in humans.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or condition (e.g., coronavirus infection), or a probe for specifically detecting a biomarker described herein. In certain embodiments, the manufacture or kit is promoted, distributed, or sold as a unit for performing the methods described herein.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Treatment by Administering Naked circRNA

The present application provides a method of treating or preventing an disease or condition in an individual, comprising administering to the individual an effective amount of the a circular RNA (circRNA) comprising a nucleic acid sequence encoding a therapeutic polypeptide, wherein the circRNA is a naked circRNA.

In some embodiments, the disease or condition is an infection. In some embodiments, the infection is a coronavirus infection. In some embodiments, the infection is SARS-COV-2 infection, optionally the SARS-COV-2 infection is caused by a SARS-COV-2 variant (e.g., a delta variant or an omicron variant or sub-variants thereof). In some embodiments, the SARS-COV-2 infection is caused by one or more SARS-COV-2 variants. In some embodiments, the SARS-COV-2 infection is caused by one or more omicron sub-variants (e.g. BA.1, BA.1.1, BA.2, BA.3, BA.4, or BA.5). In some embodiments, the SARS-COV-2 infection is caused by one or more sublineages of omicron sub-variants (e.g. BA.2.75.2, BA.4.6, BF.7, or BQ.1.1). In some embodiments, the therapeutic polypeptide is selected from the group consisting of an antigenic polypeptide, a functional protein, a receptor protein, and a targeting protein. In some embodiments, the therapeutic polypeptide is an antigenic polypeptide, for example an antigenic polypeptide present in a vaccine.

RNAs (such as circular RNAs) are typically delivered to an individual by using delivery vehicles, such as liposomes, lipid nanoparticles, or other colloidal particles. It is surprisingly found in the present application that a circRNA encoding an antigenic polypeptide, namely, a circRNA encoding an RBD antigen of a SAR-Cov-2 virus variant, when injected in naked form, could induce a high level of RBD-specific neutralizing antibodies in vivo. This allows circRNA be administered without delivery vehicles traditionally used for RNA therapeutics, such as LNPs, avoiding may of the complications associated with those delivery vehicles.

Due to their circular nature, circRNAs are particularly stable compared to many linear RNAs because they are resistant to exonucleolytic decay by the cellular exosome ribonuclease complex. The methods of the present application leverage the advantageous properties of circRNA, and allow longer intervals between different administrations. Thus, for example, in some embodiments, the circRNA is administered two or more times, such as any of two, three, four, or five times. It is believed that, when two or more doses of the circRNA are administered to an individual, it is desirable that the interval between each administration be two weeks or longer. In some embodiments, the interval between each administration is at least about four weeks, such as at least any of five, six, seven, or eight weeks. In some embodiments, the circRNA is administered with an initial dose, followed by a secondary dose at about any of 4, 6, 8, or 10 weeks.

In some embodiments, the circRNA is formulated as a solution. In some embodiments, the concentration of the circRNA in the solution is about 1 μg/mL to about 10,000 μg/mL, including for example, about 10 μg/mL to about 1,000 μg/mL, about 10 μg/mL to about 2,000 μg/mL, about 10 μg/mL to about 3,000 μg/mL, about 10 μg/mL to about 4,000 μg/mL, about 10 μg/mL to about 5,000 μg/mL, about 10 μg/mL to about 6,000 μg/mL, about 10 μg/mL to about 7,000 μg/mL, about 10 μg/mL to about 8,000 μg/mL, about 10 μg/mL to about 9,000 μg/mL, about 10 μg/mL to about 10,000 μg/mL, about 100 μg/mL to about 500 μg/mL, about 100 μg/mL to about 1,000 μg/mL, about 100 μg/mL to about 2,000 μg/mL, about 100 μg/mL to about 3,000 μg/mL, about 100 μg/mL to about 4,000 μg/mL, about 100 μg/mL to about 5,000 μg/mL, about 100 μg/mL to about 6,000 μg/mL, about 100 μg/mL to about 7,000 μg/mL, about 100 μg/mL to about 8,000 μg/mL, about 100 μg/mL to about 9,000 μg/mL, about 100 μg/mL to about 10,000 μg/mL, about 200 μg/mL to about 300 μg/mL, about 200 μg/mL to about 500 μg/mL, about 200 μg/mL to about 1,000 μg/mL, about 200 μg/mL to about 2,000 μg/mL, about 200 μg/mL to about 3,000 μg/mL, about 200 μg/mL to about 4,000 μg/mL, about 200 μg/mL to about 5,000 μg/mL, about 200 μg/mL to about 6,000 μg/mL, about 200 μg/mL to about 7,000 μg/mL, about 200 μg/mL to about 8,000 μg/mL, about 200 μg/mL to about 9,000 μg/mL, about 200 μg/mL to about 10,000 μg/mL, about 500 μg/mL to about 1,000 μg/mL, about 500 μg/mL to about 2,000 μg/mL, about 500 μg/mL to about 3,000 μg/mL, about 500 μg/mL to about 4,000 μg/mL, about 500 μg/mL to about 5,000 μg/mL, about 500 μg/mL to about 6,000 μg/mL, about 500 μg/mL to about 7,000 μg/mL, about 500 μg/mL to about 8,000 μg/mL, about 500 μg/mL to about 9,000 μg/mL, about 500 μg/mL to about 10,000 μg/mL, about 1,000 μg/mL to about 2,000 μg/mL, about 1,000 μg/mL to about 3,000 μg/mL, about 1,000 μg/mL to about 4,000 μg/mL, about 1,000 μg/mL to about 5,000 μg/mL, about 1,000 μg/mL to about 6,000 μg/mL, about 1,000 μg/mL to about 7,000 μg/mL, about 1,000 μg/mL to about 8,000 μg/mL, about 1,000 μg/mL to about 9,000 μg/mL, about 1,000 μg/mL to about 10,000.

The circRNA can be administered, for example, intravenously, intramuscularly, subcutaneously, transdermally, or via lymph node. In some embodiments, the circRNA is administered at a dose of about 1 μg to about 10,000 μg, including for example, about 10 μg to about 1,000 μg, about 100 μg to about 500 μg, about 200 μg to about 300 μg.

The circRNA for administration in some embodiments is formulated in a composition such as a vaccine or a pharmaceutical composition. In some embodiments, the circRNA composition is substantially free of a transfection agent (including for example, a polyethylenimine (PEI), a liposome, and/or a lipid nanoparticle (LNP)).

In some embodiments, the circRNA composition is a circRNA vaccine, and the circRNA vaccine is substantially free of an adjuvant. In some embodiments, the circRNA vaccine comprises an adjuvant, which may enable the vaccine to elicit a higher immune response. In some embodiments, the adjuvant is not aluminum hydroxide. In some embodiments, the circRNA composition is substantially free of aluminum hydroxide.

In some embodiments, the circRNA composition is substantially free or one or more (or all of) the following: protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents, nonliposomal transfection reagents, or any combination thereof.

In some embodiments the circRNA is administered by systemic injection into the vasculature, systemic injection into the lymph nodes, subcutaneous injection or depots, or by local injection. In some embodiments, the circRNA is administered intranasally. In some embodiments, the circRNA is administered intramuscularly or intradermally.

In some embodiments, a circRNA vaccine herein (e.g., encoding an S protein or fragment thereof of a coronavirus) is administered via intramuscular (i.m) injection. In some embodiments, one or more doses of the circRNA vaccine are administered. In some embodiments, two or more doses of the circRNA vaccine are administered. In some embodiments, the interval between doses is at least 2 weeks (e.g., at least about any of 3, 4, 5, 6, 7, or 8 weeks). In some embodiments, the method comprises administering a first dose of the circRNA vaccine and administering a second dose of the circRNA vaccine after 2 weeks or more, for example about any of 3, 4, 5, 6, 7, or 8 weeks.

In some embodiments, the circRNA functions as an adjuvant. As an example, RNA-sensing in the cytoplasm can trigger innate immunity, and innate immune signaling is known to contribute to adaptive immunity by diverse routes. Thus, the circRNA comprising the antigenic polypeptide or a second circRNA (e.g., a circRNA that does not encode a polypeptide) can be used as an adjuvant for boosting the adaptive immune response to the antigenic polypeptide.

In some embodiments, the circRNA compositions may be administered with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

### III. Therapeutic Circular RNA

The method of the present application involves administration of circular RNAs (circRNAs) encoding polypeptides, such as therapeutic polypeptides, including any one of therapeutic polypeptides described in Section A, “Therapeutic polypeptides” below.

In some embodiments, the circRNA comprises a nucleic acid sequence encoding a therapeutic polypeptide, wherein the therapeutic polypeptide is selected from the group consisting of an antigenic polypeptide, a functional protein, a receptor protein, and a targeting protein.

In some embodiments, the circRNA is stable for at least 7, 8, 9, 10, 11, 12, 13, 14, 15, 16, 17, 18, 19, 20, or 20 days when stored at 4° C. or at room temperature. In some embodiments, the circRNA is stable for at least 7 days when stored at 4° C. or at room temperature. In some embodiments, the circRNA is stable for at least 14 days when stored at 4° C. or at room temperature. In some embodiments, the circRNA is stable for at least 30 days when stored at 4° C. In some embodiments, the circRNA is less than 40% degraded after storage at room temperature for 14 days. In some embodiments, the circRNA is less than 40% degraded after storage at 4° C. for 14 days. In some embodiments, the circRNA is less than 30% degraded after storage at 4° C. or at room temperature for 14 days. In some embodiments, the circRNA is less than 20% degraded after storage at 4° C. or at room temperature for 14 days. In some embodiments, the circRNA is less than 10% degraded after storage at 4° C. or at room temperature for 14 days. In some embodiments, the circRNA is less than 40% degraded after storage at 4° C. or at room temperature for 7 days. In some embodiments, the circRNA is less than 30% degraded after storage at 4° C. or at room temperature for 7 days. In some embodiments, the circRNA is less than 20% degraded after storage at 4° C. or at room temperature for 7 days. In some embodiments, the circRNA is less than 10% degraded after storage at 4° C. or at room temperature for 7 days. In some embodiments, the circRNA is less than 5% degraded after storage at 4° C. or at room temperature for 7 days.

In some embodiments, the circRNA comprises: (a) a nucleic acid sequence encoding a therapeutic polypeptide, wherein the therapeutic polypeptide is selected from the group consisting of an antigenic polypeptide, a functional protein, a receptor protein, and a targeting protein, and (b) an internal ribosomal entry site (IRES) sequence, wherein the IRES sequence is operably linked to the nucleic acid sequence encoding the therapeutic polypeptide. In some embodiments, the nucleic acid sequence further encodes a SP (e.g., human tPA or IgE SP) fused to the N-terminus of the therapeutic polypeptide. In some embodiments, the circRNA further comprises a Kozak sequence operably linked to the nucleic acid sequence encoding the therapeutic polypeptide. In some embodiments, the circRNA comprises a nucleic acid sequence comprising from the 5′ end to the 3′ end: the IRES sequence, the Kozak sequence, and the nucleic acid sequence encoding the therapeutic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase).

In some embodiments, the circRNA comprises: (a) a nucleic acid sequence encoding a therapeutic polypeptide, wherein the therapeutic polypeptide is selected from the group consisting of an antigenic polypeptide, a functional protein, a receptor protein, and a targeting protein; (b) an IRES sequence, wherein the IRES sequence is operably linked to the nucleic acid sequence encoding the therapeutic polypeptide; and (c) an in-frame 2A peptide coding sequence operably linked to the 3′ end of the nucleic acid sequence encoding the therapeutic polypeptide. In some embodiments, the nucleic acid sequence further encodes a SP (e.g., human tPA or IgE SP) fused to the N-terminus of the therapeutic polypeptide. In some embodiments, the circRNA further comprises a Kozak sequence operably linked to the nucleic acid sequence encoding the therapeutic polypeptide. In some embodiments, the circRNA comprises a nucleic acid sequence comprising from the 5′ end to the 3′ end: the IRES sequence, the Kozak sequence, the nucleic acid sequence encoding the therapeutic polypeptide, and the in-frame 2A peptide coding sequence. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase).

In some embodiments, the circRNA comprises: (a) a nucleic acid sequence encoding a therapeutic polypeptide, wherein the therapeutic polypeptide is selected from the group consisting of an antigenic polypeptide, a functional protein, a receptor protein, and a targeting protein; and (b) an m6A modification motif sequence operably linked to the nucleic acid sequence encoding the therapeutic polypeptide. In some embodiments, the nucleic acid sequence further encodes a SP (e.g., human tPA or IgE SP) fused to the N-terminus of the therapeutic polypeptide. In some embodiments, the circRNA further comprises a Kozak sequence operably linked to the nucleic acid sequence encoding the therapeutic polypeptide. In some embodiments, the circRNA comprises a nucleic acid sequence comprising from the 5′ end to the 3′ end: the m6A modification motif sequence, the Kozak sequence, and the nucleic acid sequence encoding the therapeutic polypeptide. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase).

In some embodiments, the circRNA comprises a nucleic acid sequence encoding an antigenic polypeptide. In some embodiments, the antigenic polypeptide is a protein or fragment thereof of an infectious agent. In some embodiments, the infectious agent is a virus. In some embodiments, the virus is a coronavirus. In some embodiments, the coronavirus is selected from the group consisting of SARS-COV, MERS-COV, and SARS-COV-2. In some embodiments, the coronavirus is SARS-COV-2. The circRNA may comprise any one of the circRNA expression and/or circularization elements described in Section B, “Additional circRNA expression and circularization elements” below.

In some embodiments, the circRNA comprises a nucleic acid sequence encoding a receptor protein. In some embodiments, the receptor protein is a soluble receptor comprising an extracellular domain of a naturally occurring receptor. In some embodiments, the receptor protein is a receptor of an infectious agent (e.g., a virus such as a coronavirus). In some embodiments, the receptor is an ACE2 receptor, such as a soluble ACE2 receptor. In some embodiments, the receptor is a high-affinity mutant ACE2 receptor. The circRNA may comprise any one of the circRNA expression and/or circularization elements described in Section B, “Additional circRNA expression and circularization elements” below.

In some embodiments, the circRNA comprises a nucleic acid sequence encoding a targeting protein. In some embodiments, the targeting protein is an antibody. In some embodiments, the antibody is a neutralizing antibody, e.g., a neutralizing antibody targeting a coronavirus such as SARS-COV-2. In some embodiments, the targeting protein is a therapeutic antibody. The circRNA may comprise any one of the circRNA expression and/or circularization elements described in Section B, “Additional circRNA expression and circularization elements” below.

In some embodiments, the circRNA is provided as a circular RNA (circRNA) vaccine comprising a circRNA comprising a nucleic acid sequence encoding an antigenic polypeptide comprising a Spike(S) protein or a fragment thereof of a coronavirus (e.g., SARS-COV, MERS-COV, or SARS-COV-2).

In some embodiments, the circRNA vaccine comprises a circRNA comprising a nucleic acid sequence encoding an antigenic polypeptide comprising an S protein or a fragment thereof of SARS-COV-2.

In some embodiments, the circRNA vaccine comprises a circRNA comprising a nucleic acid sequence encoding an antigenic polypeptide comprising: (a) an S protein or a fragment thereof of a coronavirus (e.g., SARS-COV, MERS-COV, or SARS-COV-2); and (b) a multimerization domain. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises an amino acid sequence set forth in SEQ ID NOs: 3-4. In some embodiments, the multimerization domain is fused to the RBD domain of the S protein via a peptide linker, e.g., a peptide linker comprising the amino acid sequence of SEQ ID NO: 5.

In some embodiments, the circRNA vaccine comprises a circRNA comprising a nucleic acid sequence encoding an antigenic polypeptide comprising a receptor-binding domain (RBD) of an S protein of a coronavirus (e.g., SARS-COV2). In some embodiments, the RBD comprises amino acid residues 319 to 542 of a full-length S protein of SARS-COV-2, wherein the numbering is based on SEQ ID NO: 1. In some embodiments, the RBD comprises the amino acid sequence of SEQ ID NO: 2. In some embodiments, the RBD comprises the amino acid sequence of SEQ ID NO: 63.

In some embodiments, the circRNA vaccine comprises a circRNA comprising a nucleic acid sequence encoding an antigenic polypeptide comprising: (a) a RBD of an S protein fragment of a coronavirus (e.g., SARS-COV, MERS-COV, or SARS-COV-2) and (b) a multimerization domain. In some embodiments, the RBD comprises amino acid residues 319 to 542 of a full-length S protein of SARS-COV-2, wherein the numbering is based on SEQ ID NO: 1. In some embodiments, the RBD comprises the amino acid sequence of SEQ ID NO: 2. In some embodiments, the RBD comprises the amino acid sequence of SEQ ID NO: 63. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises an amino acid sequence set forth in SEQ ID NOs: 3-4. In some embodiments, the multimerization domain is fused to the RBD domain of the S protein via a peptide linker, e.g., a peptide linker comprising the amino acid sequence of SEQ ID NO: 5.

In some embodiments, the circRNA vaccine comprises a circRNA comprising a nucleic acid sequence encoding an antigenic polypeptide comprising an S2 region of an S protein of a coronavirus (e.g., SARS-COV2). In some embodiments, the S2 region comprises amino acid residues 686 to 1273 of a full-length S protein of SARS-COV-2, wherein the numbering is based on SEQ ID NO: 1. In some embodiments, the S2 region comprises one or more mutations (e.g., K986P and V987P) that stabilize a pre-fusion conformation of the S protein. In some embodiments, the S2 region comprises the amino acid sequence of SEQ ID NO: 6 or 7.

In some embodiments, the circRNA vaccine comprises a circRNA comprising a nucleic acid sequence encoding an antigenic polypeptide comprising amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an amino acid sequence selected from the group consisting of SEQ ID NOs: 8-10, 62-63, and 96-97.

In some embodiments, the circRNA vaccine comprises a circRNA comprising: (a) a nucleic acid sequence encoding an antigenic polypeptide comprising an S protein or a fragment thereof of a coronavirus (e.g., SARS-COV-2), and (b) an internal ribosomal entry site (IRES) sequence, wherein the IRES sequence is operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, and the nucleic acid sequence encoding the antigenic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 11-15.

In some embodiments, the circRNA vaccine comprises a circRNA comprising: (a) a nucleic acid sequence encoding an antigenic polypeptide comprising an S protein or a fragment thereof of a coronavirus (e.g., SARS-COV-2); (b) an IRES sequence, wherein the IRES sequence is operably linked to the nucleic acid sequence encoding the antigenic polypeptide; and (c) an in-frame 2A peptide coding sequence operably linked to the 3′ end of the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, the nucleic acid sequence encoding the antigenic polypeptide, and the in-frame 2A peptide coding sequence. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 11-15.

In some embodiments, the circRNA vaccine comprises a circRNA comprising: (a) a nucleic acid sequence encoding an antigenic polypeptide comprising an S protein or a fragment thereof of a coronavirus (e.g., SARS-COV-2), and (b) an m6A modification motif sequence operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the m6A modification motif sequence, the Kozak sequence, the SP, and the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the circRNA comprises a nucleic acid sequence selected from the group consisting of SEQ ID NOs: 11-15.

The circRNA described herein may also be provided in a cocktail composition comprising a plurality of circRNAs each comprising a nucleic acid sequence encoding an antigenic polypeptide, a receptor protein of an infectious agent, or a targeting protein (e.g., an antibody such as a neutralizing antibody). In some embodiments, the plurality of circRNA encode antigenic polypeptides that are different with respect to each other, such as different mutants of an antigenic polypeptide (e.g., S protein or fragment thereof). In some embodiments, the plurality of circRNA encode receptor proteins that are different with respect to each other, such as different mutants of a receptor protein (e.g., ACE2). In some embodiments, the plurality of circRNA encode targeting proteins that are different with respect to each other, such as different antibodies (e.g., neutralizing antibodies).

### A. Therapeutic Polypeptides

In some aspects, the circRNA comprises a therapeutic polypeptide. In some embodiments, the therapeutic polypeptide is an antigenic polypeptide, a functional protein, a receptor protein or a targeting protein (e.g., an antibody).

In some embodiments, the nucleic acid sequence may be codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the IRES.

i. Antigenic Polypeptides

The circRNA described herein may encode an antigenic polypeptide. In some embodiments, the antigenic polypeptide comprises a Spike(S) protein or a fragment thereof of a coronavirus, such as any one of the S proteins or fragments thereof as described in the “Spike protein or fragment thereof” subsection below. In some embodiments, the antigenic polypeptide comprises a multimerization domain, such as a native multimerization domain of the S protein, or an exogenous multimerization domain. Suitable multimerization domains are described in the “Multimerization domain” subsection below. The S protein or fragment thereof may be fused to the multimerization domain via a peptide linker, such as any one of the peptide linkers described in the “peptide linker” subsection below.

An antigenic polypeptide comprises at least one epitope recognizable by a T cell receptor (TCR). In some embodiments, the antigenic polypeptide is a full-length protein or a fragment thereof, or an antigenic fusion protein that can trigger an immune response in a subject. In some embodiments, the antigenic polypeptide is a short peptide of no more than 100 amino acids long. The antigenic polypeptide can be a naturally derived peptide fragment from a protein antigen containing one or more epitopes, or an artificially designed peptide with one or more natural epitope sequences, wherein a peptide linker may optionally be placed in between adjacent epitope sequences. In some embodiments, the antigenic polypeptide comprises a single epitope of an antigenic protein. In some embodiments, the antigenic polypeptide comprises about any one of 1, 2, 3, 4, 5, 10 or more epitopes from a single antigenic protein. In some embodiments, the antigenic polypeptide comprises epitopes from a plurality (e.g., 2, 3, 4, 5, 10 or more) of different antigenic proteins. In some embodiments, the antigenic polypeptide comprises a Major Histocompatibility Complex (MHC) class I-restricted epitope. In some embodiments, the antigenic polypeptide comprises a MHC class II-restricted epitope. In some embodiments, the antigenic polypeptide comprises both MHC class I-restricted and MHC class II-restricted epitopes.

In some embodiments, the antigenic polypeptide is an antigenic protein or fragment thereof or a variant thereof from a pathogenic agent, such as a bacterium or a virus. In some embodiments, the antigenic polypeptide is an antigenic protein or fragment of a coronavirus, such as SARS-COV2, including variants thereof.

In some embodiments, the antigenic polypeptide is an antigenic protein or fragment thereof or a variant thereof of a self antigen, such as an antigen involved in a disease or condition. In some embodiments, the antigenic polypeptide is a tumor antigen peptide. Tumor antigen peptide sequences are known in the art and can be found at public databases, such as the Cancer Antigenic Peptide Database (van der Bruggen P et al. (2013) “Peptide database: T cell-defined tumor antigens.” Cancer Immunity. URL: caped.icp.ucl.ac.be, incorporated herein by reference in its entirety). The coding RNA sequence in the linear RNA or circRNA described herein may encode any of the known tumor antigen peptides or combinations thereof. In some embodiments, the antigenic polypeptide comprises an epitope of a tumor associated antigen (TAA). In some embodiments, the antigenic polypeptide comprises an epitope of a tumor specific antigen. In some embodiments, the antigenic polypeptide comprises an epitope of a neoantigen, i.e., newly acquired and expressed antigens present in tumor cells of an individual.

In some embodiments, the amino acid sequences of one or more epitope peptides are predicted based on the sequence of the antigen protein (including neoantigens) using a bioinformatics tool for T cell epitope prediction. Exemplary bioinformatics tools for T cell epitope prediction are known in the art, for example, see Yang X. and Yu X. (2009) “An introduction to epitope prediction methods and software” Rev. Med. Virol. 19 (2): 77-96, incorporated herein by reference in its entirety. In some embodiments, the sequence of the antigen protein is known in the art or available in public databases. In some embodiments, the sequence of the antigen protein (including neoantigens) is determined by sequencing a sample (such as a tumor sample) of the individual being treated.

In some embodiments, the antigenic polypeptide comprises a Spike(S) protein or a fragment thereof of a coronavirus, such as a SARS-COV, MERS-COV, or SARS-COV-2 virus. In some embodiments, the antigenic polypeptide is a full-length S protein. In some embodiments, the antigenic polypeptide is a fragment of a naturally occurring S protein. In some embodiments, the antigenic polypeptide comprises a Spike(S) protein or a fragment thereof of SARS-COV-2.

In some embodiments, the antigenic polypeptide comprises a variant of an S protein or fragment thereof of a coronavirus. In some embodiments, the antigenic polypeptide comprises a naturally occurring variant of an S protein or fragment thereof of a coronavirus (e.g., SARS-COV-2). Variants of the SARS-COV-2 genome have been described. See, for example, Forster et al. (2020). Phylogenetic network analysis of SARS-COV-2 genomes. PNAS 117 (17) 9241-9243, which is incorporated herein by reference in its entirety. In some embodiments, the antigenic polypeptide comprises a variant of an S protein or fragment thereof that confers a fitness advantage to a coronavirus, such as enhanced infectivity. In some embodiments, the antigenic polypeptide comprises an S protein or fragment thereof of SARS-COV-2 having a D614G mutation. In some embodiments, the antigenic polypeptide is capable of eliciting an immune response in an individual against different strains and variants of a coronavirus, such as SARS-COV-2 variants. In some embodiments, the antigenic polypeptide is capable of eliciting an immune response in an individual against a specific strain or variant of a coronavirus.

In some embodiments, the antigenic polypeptide comprises a receptor-binding domain (RBD) of an S protein of a coronavirus (e.g., SARS-COV2). In some embodiments, the RBD comprises amino acid residues 319 to 542 of a full-length S protein of SARS-COV-2, wherein the numbering is based on SEQ ID NO: 1 In some embodiments, the RBD comprises an amino acid sequence having at least about 80% (e.g., at least about 85%, 90%, 95%, 98% or more, or 100%) sequence identity to the amino acid sequence of SEQ ID NO: 2. In some embodiments, the RBD comprises an amino acid sequence having at least about 80% (e.g., at least about 85%, 90%, 95%, 98% or more, or 100%) sequence identity to the amino acid sequence of SEQ ID NO: 63.

In some embodiments, the antigenic polypeptide comprises an S2 region of an S protein of a coronavirus (e.g., SARS-COV2). In some embodiments, the S2 region comprises amino acid residues 686 to 1273 of a full-length S protein of SARS-COV-2, wherein the numbering is based on SEQ ID NO: 1. In some embodiments, the S2 region comprises an amino acid sequence having at least about 80% (e.g., at least about 85%, 90%, 95%, 98% or more, or 100%) sequence identity to the amino acid sequence of SEQ ID NO: 6. In some embodiments, the S2 region comprises one or more mutations that stabilize a pre-fusion conformation of the S protein. In some embodiments, the S2 region comprises K986P and V987P mutations. In some embodiments, the S2 region comprises an amino acid sequence having at least about 80% (e.g., at least about 85%, 90%, 95%, 98% or more, or 100%) sequence identity to the amino acid sequence of SEQ ID NO: 7.

In some embodiments, the antigenic polypeptide comprises both an RBD and an S2 region of an S protein of a coronavirus (e.g., SARS-COV2). In some embodiments, the antigenic polypeptide comprises an amino acid sequence having at least about 80% (e.g., at least about 85%, 90%, 95%, 98% or more, or 100%) sequence identity to the amino acid sequence of SEQ ID NO: 1.

In some embodiments, the antigenic polypeptide comprises a Spike(S) protein fragment of a coronavirus (e.g., SARS-COV, MERS-COV, or SARS-COV-2) and a multimerization domain, which can be operably linked to the S protein fragment. In some embodiments, the multimerization domain is a C-terminal Foldon (Fd) domain of a T4 fibritin protein that mediates trimerization of the T4 fibritin protein. In some embodiments, the multimerization domain is a GCN-4 based isoleucine zipper domain. In some embodiments, the multimerization domain comprises the amino acid sequence as set forth in SEQ ID NO: 3 or 4. In some embodiments, the multimerization domain is fused to the S protein fragment via a peptide linker. In some embodiments, the antigenic polypeptide comprises a RBD domain of an S protein fused to a multimerization domain via a peptide linker. In some embodiments, the peptide linker comprises the amino acid sequence of SEQ ID NO: 5.

In some embodiments, the antigenic polypeptide comprises a Spike(S) protein or a fragment thereof of SARS-COV-2 fused to a multimerization domain. In some embodiments, the antigenic polypeptide comprises an S protein fragment fused to a C-terminal Foldon (Fd) domain (e.g., SEQ ID NO: 3) of a T4 fibritin protein that mediates trimerization of the T4 fibritin protein (e.g., SEQ ID NO: 4). In some embodiments, the antigenic polypeptide comprises an S protein fragment fused to a GCN-4 based isoleucine zipper domain. In some embodiments, the antigenic polypeptide comprises a receptor-binding domain (RBD) of an S protein of SARS-COV-2 fused to a multimerization domain via a peptide linker. In some embodiments, the peptide linker comprises the amino acid sequence of SEQ ID NO: 5.

The antigenic polypeptide may comprise a signal peptide (SP). In some embodiments, the SP is fused to the N-terminus of the S protein or fragment thereof. In non-limiting examples, the signal peptide is the signal sequence and propeptide from human tissue plasminogen activator (tPA), the signal sequence from human IgE Immunoglobulin, or the signal peptide sequence of MHC I. In some embodiments, the signal peptide can facilitate secretion of the antigenic polypeptide encoded by the circRNA vaccine.

In some embodiments, the circRNA comprises an in-frame 2A peptide coding sequence operably linked to the 3′ end of the nucleic acid sequence encoding the antigenic polypeptide. In some embodiments, the circRNA does not comprise a stop codon at the 3′ end of the nucleic acid sequence encoding the antigenic polypeptide. In some embodiments, the in-frame 2A peptide coding sequence replaces the stop codon. In some embodiments, the circRNA contains no stop codon and the number of nucleotides composing the RNA is a multiple of three. In some embodiments, the circRNA having no stop codon and the number of nucleotides composing the RNA being a multiple of three allows for rolling circle translation of the circRNA. In some embodiments, the 2A peptide coding sequence allows for rolling circle translation of the circRNA. In some embodiments, the 2A peptide allows cleavage of a polypeptide generated by rolling circle translation into monomeric polypeptide sequences. In non-limiting examples, the 2A peptide coding sequence encodes a P2A or T2A peptide, such as the sequence set forth in SEQ ID NO: 44 or 45.

The nucleic acid sequences encoding the antigenic polypeptides described herein may be codon-optimized. In some embodiments, the circRNA comprises a nucleic acid sequence having at least about 80% (e.g., at least about 85%, 90%, 95%, 98% or more, or 100%) sequence identity to a nucleic acid sequence selected from the group consisting of SEQ ID NOs: 11-15 and SEQ ID NOs: 48-49.

### Spike Protein or Fragment Thereof.

The circRNA described herein in some embodiments comprises a nucleic acid sequence encoding an antigenic polypeptide comprising a Spike(S) protein or a fragment thereof of a coronavirus (e.g., SARS-COV-2, MERS-COV, or SARS-COV). Sequences of S proteins of coronaviruses are known in the art, including, for example, NCBI RefSeq ID: YP_009047204.1 (MERS-COV), GenBank Accession number: AAT74874 (SARS-COV), or NCBI RefSeq ID: YP_009724390 (SARS-COV-2, provided as SEQ ID NO: 1 of the present application).

In some embodiments, the S protein or fragment thereof comprises amino acid residues 2-1273 of a full-length S protein of SARS-COV-2, wherein the numbering is based on SEQ ID NO: 1. In some embodiments, the S protein or fragment thereof comprises a deletion of amino acid residues 681-684. In some embodiments, the S protein or fragment thereof comprises at least one point mutation in the S2 region, for example, a K986P, V987P, F817P, A892P, A899P, or A942P mutation or combinations thereof. In some embodiments, the S protein of fragment thereof comprises at least one mutation selected from A222V, E406W, K417N, K417T, N439K, L452R, L452Q, L455N, L478K, E484K, Q493F, F490S, N501Y, A570D, D614G, P681H, A701V, T716I, S982A, or combinations thereof. In some embodiments, the S protein or fragment thereof comprises a N501Y point mutation. In some embodiments, the S protein or fragment thereof comprises K417N, E484K, and/or N501Y point mutations. In some embodiments, the S protein or fragment thereof comprises an E484K point mutation. In some embodiments, the S protein or fragment thereof comprises K417T, E484K, and N501Y point mutations. In some embodiments, the S Protein or fragment thereof of SARS-COV-2 comprises K986P and V987P point mutations, either alone or in combination with a deletion of amino acid residues 681-684. In some embodiments, the S protein or fragment thereof comprises an amino acid sequence set forth in any one of SEQ ID NO: 1-2, SEQ ID NO: 6-10, or SEQ ID NO: 63. In some embodiments, the S protein or fragment thereof comprises an amino acid sequence set forth in SEQ ID NO: 2. In some embodiments, the S protein or fragment thereof comprises an amino acid sequence set forth in SEQ ID NO: 63.

In some embodiments, the S protein or fragment thereof is an Alpha (B.1.1.7), Beta (B.1.351, B.1.351.2, B.1.351.3), Delta (B.1.617.2, AY.1, AY.2, AY.3), or Gamma (P.1, P.1.1, P.1.2) S protein or fragment thereof. In some embodiments, the S protein or fragment thereof comprises two, three, four, five, or more mutations selected from the group consisting of T19R, V70F, T95I, G142D, E156-, F157-, R158G, A222V, W258L, K417N, L452R, T478K, D614G, P681R, and D950N, wherein the amino acid numbering is based on SEQ ID NO. 1. In some embodiments, the S protein or fragment thereof comprises an RBD comprising the S protein or fragment thereof comprises one, two, or three of the or more mutations selected from the group K417N, L452R, and T478K, wherein the amino acid numbering is based on SEQ ID NO: 1. In some embodiments, the S protein or fragment thereof comprises two, three, four, five, or more mutations selected from the group consisting of residue 69 deletion, residue 70 deletion, residue 144 deletion, E484K, S494P, N501Y, A570D, D614G, P681H, T716I, S982A, D1118H, and K1191N, wherein the amino acid numbering is based on SEQ ID NO: 1. In some embodiments, the S protein or fragment thereof comprises an RBD domain comprising one, two, or three of the mutations selected from the group consisting of E484K, S494P, and N501Y, wherein the amino acid numbering is based on SEQ ID NO: 1. In some embodiments, the S protein or fragment thereof comprises one, two, three, four, five, or more mutations selected from the group consisting of D80A, D215G, 241del, 242del, 243del, K417N, E484K, N501Y, D614G, and A701V, wherein the amino acid numbering is based on SEQ ID NO: 1. In some embodiments, the S protein or fragment thereof comprises an RBD comprising one, two, or three of the mutations selected from the group consisting of K417N, E484K, and N501Y, wherein the amino acid numbering is based on SEQ ID NO: 1. In some embodiments, the S protein or fragment thereof comprises one, two, three, four, five, or more mutations selected from the group consisting of L18F, T20N, P26S, D138Y, R190S, K417T, E484K, N501Y, D614G, H655Y, and T1027I, wherein the amino acid numbering is based on SEQ ID NO: 1. In some embodiments, the S protein or fragment thereof comprises an RBD domain comprising one, two, or three of the mutations selected from the group consisting of K417T, E484K, and N501Y, wherein the amino acid numbering is based on SEQ ID NO. 1. In some embodiments, the S protein or fragment thereof comprises an RBD domain comprising one, two, or three of the mutations selected from the group consisting of E484K, N501Y, and L452R mutations, wherein the amino acid numbering is based on SEQ ID. NO: 1.

In some embodiments, the S protein or fragment thereof comprises an N-terminal domain (NTD) of an S protein of a coronavirus (e.g., SARS-COV-2, MERS-COV, or SARS-COV).

In some embodiments, the S protein or fragment thereof comprises an amino acid sequence having about 80%, at least 85%, at least about 90%, at least about 95%, at least about 98% or more sequence identity to a wild-type S protein or a fragment thereof of a coronavirus, or with any one of the sequences set forth in SEQ ID NOs: 1-2, SEQ ID NOs: 6-10, SEQ ID NOS: 62-63, and SEQ ID Nos: 96-97.

### RBD Domain

In some embodiments, the S protein or fragment thereof described herein comprises a receptor-binding domain (RBD) of the S protein. In some embodiments, the RBD comprises amino acid residues 319 to 542 of a full-length S protein of SARS-COV-2, wherein the numbering is based on SEQ ID NO: 1 In some embodiments, the RBD comprises the amino acid sequence of SEQ ID NO: 2. In some embodiments, the RBD comprises a sequence having about 80%, at least 85%, at least about 90%, at least about 95%, at least about 98% or more sequence identity with the amino acid sequence of SEQ ID NO: 2. In some embodiments, the RBD comprises a sequence having about 80%, at least 85%, at least about 90%, at least about 95%, at least about 98% or more sequence identity with the amino acid sequence of SEQ ID NO: 63. In some embodiments, the RBD is linked to a multimerization domain. In some embodiments, the RBD is fused to a multimerization domain by a flexible peptide linker.

### S2 Region

In some embodiments, the S protein or fragment thereof comprises an S2 region of the S protein. In some embodiments, the S2 region comprises amino acid residues 686 to 1273 of a full-length S protein of SARS-COV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises K986P and V987P mutations, for example, as in the sequence set forth in SEQ ID NO: 7. In some embodiments, the S2 region comprises a single point mutation, for example, a K986P, V987P, F817P, A892P, A899P or A942P mutation. In some embodiments, the S2 region comprises a combination of point mutations including K986P, V987P, F817P, A892P, A899P or A942P. In some embodiments, the S2 region comprises the wild type sequence of an S protein of a coronavirus, such as the sequence of SEQ ID NO: 6, or a sequence having about 80%, at least 85%, at least about 90%, at least about 95%, at least about 98% or more sequence identity with the amino acid sequence of SEQ ID NO: 6.

### Multimerization Domain

In some embodiments, the antigenic polypeptide further comprises a multimerization domain, such as a dimerization domain, a trimerization domain, or a domain that mediates formation of higher order multimers. In some embodiments, the multimerization domain is a trimerization domain. In non-limiting examples, the multimerization domain comprises a C-terminal Foldon (Fd) domain of a T4 fibritin protein, wherein the C-terminal Foldon domain is the domain that mediates trimerization of the T4 fibritin protein, such as the amino acid sequence set forth in SEQ ID NO: 3. In another example, the multimerization domain comprises a GCN4-based isoleucine zipper (IZ) domain based on the trimerization domain of the GCN4 transcriptional activator from Saccharomyces cerevisiae, such as the amino acid sequence set forth in SEQ ID NO: 4. In some embodiments, the multimerization domain has about 80%, at least 85%, at least about 90%, at least about 95%, at least about 98% or more sequence identity with the amino acid sequence of SEQ ID NO: 3 or SEQ ID NO: 4. In some embodiments, the GCN4 IZ domain or T4 fibritin Fd domain can be modified to reduce their immunogenicity according to known techniques in the art. For example, the GCN4 IZ domain can be modified with N-linked glycosylation sites to reduce its immunogenicity (Sliepen et al. Immunosilencing a Highly Immunogenic Protein Trimerization Domain. The Journal of Biol. Chem. Vol. 290, No. 12, pp. 7436-7442, incorporated herein by reference in its entirety). In some embodiments, the multimerization domain is fused to the N-terminus of the S protein or fragment thereof. In some embodiments, the multimerization domain is fused to the C-terminus of the S protein or fragment thereof.

ii. Targeting Proteins

In some embodiments, the therapeutic polypeptide described herein is a targeting protein. In some embodiments, the targeting protein is an antibody or an antigen-binding fragment thereof.

In some embodiments, the therapeutic polypeptide is an antibody. In some embodiments, the therapeutic polypeptide is a neutralizing antibody, i.e., an antibody that blocks an interaction between a protein and its binding partner. In some embodiments, the antibody inhibits activity of a protein, e.g., by blocking binding of the protein to a binding partner. In some embodiments, the targeting protein is a therapeutic antibody. In some embodiments, the antibody is a checkpoint inhibitor, e.g., an antibody inhibitor of CTLA-4, PD-1, or PD-L1. In some embodiments, the antibody can be an antibody against a viral protein or a receptor that binds to a viral protein.

The antibody can be an antigen-binding fragment of an antibody, e.g., a portion or fragment of an intact or complete antibody having fewer amino acid residues than the intact or complete antibody, which is capable of binding to an antigen or competing with the intact antibody (i.e., the intact antibody from which the antigen-binding fragment is derived) for binding to an antigen. Antigen-binding fragments can be prepared by recombinant DNA techniques, or by enzymatic or chemical cleavage of intact antibodies. Antigen binding fragments include, but are not limited to, Fab′, F(ab′)2Fv, single chain Fv (scFv), single chain Fab, diabody (diabody), single domain antibody (sdAb, nanobody), camel Ig, Ig NAR, F (ab)′3Fragment, bis-scFv, (scFv)2Minibodies, diabodies, triabodies, tetradiabodies, and disulfide stabilized Fv proteins (“dsFv”). In some embodiments, the neutralizing antibody can be a genetically engineered antibody, such as a chimeric antibody (e.g., humanized murine antibodies), heteroconjugate antibody (e.g., bispecific antibodies), or antigen-binding fragments thereof.

In some embodiments, the antibody is a neutralizing antibody that binds to a viral protein. In some embodiments, the antibody is a neutralizing antibody that binds to a receptor for a viral protein. In some embodiments, the antibody binds to a receptor that is required for viral entry into a cell (e.g., an ACE2 receptor). In some embodiments, the antibody is a neutralizing antibody (nAb) that binds to the S protein of a coronavirus and prevents or reduces its ability to infect cells. In some embodiments, the coronavirus is SARS-COV-2. In some embodiments, the nAb is a monoclonal antibody (mAb), a functional antigen-binding fragment (Fab), a single-chain variable region fragment (scFv), or a single-domain antibody (a VHH or nanobody).

In some embodiments, the nAb binds to the RBD of a S protein of a coronavirus. In some embodiments, the nAb binds to the NTD of a S protein of a coronavirus. In some embodiments, the nAB binds to the S2 region of a S protein of a coronavirus. In some embodiments, the nAb binds the S1/S2 proteolytic cleavage site of a S protein of a coronavirus. In some embodiments, the coronavirus is SARS-COV-2. In some embodiments, binding of the nAb to the S protein interferes with interaction of the RBD of the S protein with an ACE2 receptor. In some embodiments, the nAb binds to the ACE2 binding site of the RBD. In some embodiments, binding of the nAb to the S protein interferes with S2-mediated membrane fusion. In some embodiments, binding of the nAb to the S protein interferes with viral entry into the host cell.

In some embodiments, the nAb binds to a S protein comprising one or more mutations. In some embodiments, the nAb binds to a S protein or fragment thereof that comprises at least one point mutation in the S2 region, for example, a K986P, V987P, F817P, A892P, A899P or A942P mutation or combinations thereof. In some embodiments, the nAb binds to a S protein or fragment thereof that comprises at least one point mutation selected from A222V, E406W, K417N, K417T, N439K, L452R, L452Q, L455N, L478K, E484K, Q493F, F490S, N501Y, A570D, D614G, P681H, A701V, T716I, S982A, or combinations thereof. In some embodiments, the nAb binds to a S protein or fragment thereof that comprises a N501Y point mutation. In some embodiments, the nAb binds to a S protein or fragment thereof that comprises K417N, E484K, and N501Y point mutations. In some embodiments, the nAb binds to a S protein or fragment thereof that comprises an E484K point mutation. In some embodiments, the nAb binds to a S protein or fragment thereof that comprises K417T, E484K, and N501Y point mutations. In some embodiments, the nAb binds to a S Protein or fragment thereof of SARS-COV-2 that comprises K986P and V987P point mutations, either alone or in combination with a deletion of amino acid residues 681-684. In some embodiments, binding of the nAb to an S protein having any of the combinations of mutations described above (e.g., K417N, K417T, E484K, and/or N501Y) interferes with interaction of the RBD of the S protein with an ACE2 receptor. In some embodiments, binding of the nAb to an S protein any of the combinations of mutations described above (e.g., K417N, K417T, E484K, and/or N501Y) interferes with S2-mediated membrane fusion. In some embodiments, binding of the nAb to an S protein any of the combinations of mutations described above (e.g., K417N, K417T, E484K, and/or N501Y) interferes with viral entry into the host cell.

Exemplary nAbs for binding and neutralization of the S protein of SARS-COV-2 have been described, for example, in Barnes, C. O. et al. SARS-COV-2 neutralizing antibody structures inform therapeutic strategies. Nature 588, 682-687 (2020), and Chinese Patent Application No. CN111690058A, the contents of which are herein incorporated by reference in their entirety.

In some embodiments, the nAb comprises a sequence selected from SEQ ID NOs: 26-33. In some embodiments, the nAb comprises a sequence having at least 80% (e.g., at least 85%, at least 88%, at least 90%, at least 91%, at least 92%, at least 93%, at least 94%, at least 95%, at least 96%, at least 97%, at least 98%, or at least 99%) amino acid sequence identity to a sequence selected from SEQ ID NOs: 26-33.

In some embodiments, the antibody is an antibody against the S protein of SARS-COV-2. In some embodiments, the antibody comprises a sequence having at least 80% (e.g., at least 85%, at least 88%, at least 90%, at least 91%, at least 92%, at least 93%, at least 94%, at least 95%, at least 96%, at least 97%, at least 98%, at least 99%, or 100%) amino acid sequence identity to SEQ ID NO: 26. In some embodiments, the antibody comprises a sequence having at least 80% (e.g., at least 85%, at least 88%, at least 90%, at least 91%, at least 92%, at least 93%, at least 94%, at least 95%, at least 96%, at least 97%, at least 98%, at least 99%, or 100%) amino acid sequence identity to SEQ ID NO: 27. In some embodiments, the antibody comprises a sequence having at least 80% (e.g., at least 85%, at least 88%, at least 90%, at least 91%, at least 92%, at least 93%, at least 94%, at least 95%, at least 96%, at least 97%, at least 98%, at least 99%, or 100%) amino acid sequence identity to SEQ ID NO: 30.

In some embodiments, the targeting protein is not an antibody. Examples of non-antibody-based targeting proteins include, but are not limited to, a lipocalin, an anticalin (artificial antibody mimetic proteins that are derived from human lipocalins), “T-body”, a peptide (e.g., a BICYCLE™ peptide), an affibody (antibody mimetics composed of alpha helices, e.g. an three-helix bundle), a peptibody (peptide-Fc fusion), a DARPin (designd ankyrin repeat proteins, engineered antibody mimetic proteins consisting repeat motifs), an affimer, an avimer, a knottin (a protein structural motif containing 3 disulfide bridges), a monobody, an affinity clamp, an ectodomain, a receptor ectodomain, a receptor, a cytokine, a ligand, an immunocytokine, and a centryin. See, for example, Vazquez-Lombardi, Rodrigo, et al. Drug discovery today 20.10 (2015): 1271-1283, incorporated herein by reference in its entirety.

iii. Soluble Receptors

In some embodiments, the therapeutic polypeptide is a soluble receptor. Soluble receptors (sometimes referred to as soluble receptor decoys or “traps”) can comprise all or a portion of the extracellular domain of a receptor protein. In some embodiments, a nucleotide sequence encoding all or a portion of the extracellular domain of a receptor protein is operably linked to a signal peptide for secretion from cells.

In some embodiments, the soluble receptor comprises an extracellular domain of a naturally occurring receptor. In some embodiments, the soluble receptor variant comprises an engineered variant of an extracellular domain of a naturally occurring receptor, such as a variant comprising one or more mutations in the extracellular domain. In some embodiments, the soluble receptor comprises one or more mutations that increase the affinity of the soluble receptor for its ligand compared to the affinity of the naturally occurring receptor for its ligand.

In some embodiments, the soluble receptor is a fusion protein comprising one or more additional protein domains operably linked to the extracellular domain of the receptor or a variant thereof. In some embodiments, the soluble receptor comprises an Fc domain of an immunoglobulin (Ig), e.g., a human immunoglobulin. In some embodiments, the soluble receptor comprises an Fc domain of a human IgG1.

In some embodiments, the soluble receptor comprises the extracellular domain of a signaling receptor, and the soluble receptor can reduce or inhibit activity of the signaling pathway by blocking binding between the endogenous receptor and its ligand.

In some embodiments, the soluble receptor is a receptor that binds to a viral protein and/or that mediates viral entry. In some embodiments, soluble receptor is a soluble ACE2 receptor. In some embodiments, the therapeutic polypeptide is a soluble ACE2 receptor variant capable of binding to a S protein of a coronavirus. In some embodiments, the soluble ACE2 can have a great advantage over antibodies due to resistance to escape mutations. The virus with escape mutation from sACE2 should have limited binding affinity to cell surface native ACE2 receptors, leading to a diminished or eliminated virulence.

In some embodiments, the ACE2 receptor fragment is engineered to have higher affinity to a S protein of a coronavirus. In some embodiments, the soluble ACE2 receptor variant is capable of binding to a S protein of a coronavirus and blocking or reducing binding of the S protein to an endogenous ACE2 receptor. In some embodiments, the soluble ACE2 receptor variant binds to the receptor binding domain (RBD) of the S protein. In some embodiments, the ACE2 receptor variant is enzymatically active. In other embodiments, the ACE2 receptor variant is enzymatically inactive.

In some embodiments, the soluble ACE2 receptor variant comprises the soluble extracellular domain of wild-type (WT) human recombinant ACE2 (APN01). APN01 has been found to be safe in healthy volunteers and in a small cohort of patients with acute respiratory distress syndrome by virtue of ACE2's intrinsic angiotensin-converting activity, which is not required for viral entry. APN01 has been investigated in phase 2 clinical trials in Europe for treatment of SARS-COV-2 (NCT04335136). In some embodiments, the soluble ACE2 receptor variant comprises one or more mutations in the extracellular domain of human ACE2. In some embodiments, the soluble ACE2 receptor variant is engineered via affinity maturation to have increased binding affinity to the RBD of the S protein. For example, a nucleotide sequence encoding a wild-type extracellular domain of ACE2 may be subjected to one or more cycles of random mutation and cell sorting to identify ACE2 variants having a higher affinity for the RBD of the S protein wild-type ACE2.

In some embodiments, the soluble ACE2 receptor variant comprises a sequence having at least 80% (e.g., at least 85%, at least 88%, at least 90%, at least 91%, at least 92%, at least 93%, at least 94%, at least 95%, at least 96%, at least 97%, at least 98%, at least 99%, or 100%) amino acid sequence identity to SEQ ID NO: 34 or 35.

In some embodiments, the soluble ACE2 receptor variant is a fusion protein, e.g., a fusion of the extracellular ACE2 receptor domain to the Fc region of the human IgG1.

In some embodiments, the K_{D} of the soluble ACE2 receptor variant for the RBD of the S protein is about 15-20 nM. In some embodiments, the K_{D} of the soluble ACE2 receptor variant for the RBD of the S protein is less than 15 nM, less than 10 nM, less than 5 nM, less than 1 nM, less than 500 pM, less than 250 pM, less than 200 pM, or less than 150 pM.

Soluble ACE2 receptor variants have been described, for example in Haschke M et al., Clin Pharmacokinet. 2013 September; 52 (9): 783-92; Glasgow A et al., Proceedings of the National Academy of Sciences November 2020, 117 (45) 28046-28055; and Higuchi Y. et al., bioRxiv 2020. 09.16.299891, the contents of which are herein incorporated by reference in their entirety.

iv. Functional Proteins

In some embodiments, the therapeutic polypeptide can be any polypeptide that is capable of being expressed by target cells (e.g., human or mouse cells) for the production (and in certain instances, the excretion) of a functional enzyme or protein as disclosed, for example, in International Application No. PCT/US2010/058457 and WO2020237227, the contents of which are herein incorporated by reference in their entirety. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

In some embodiments, the therapeutic polypeptide comprises a protein such as IDUA, OTC, FAH, miniDMD, DMD, p53, PTEN, COL3A1, BMPR2, AHI1, FANCC, MYBPC3, ILRG2, or ARG1, wherein deficiency of functional protein is associated with a disease or disorder.

In some embodiments, the therapeutic polypeptide comprises a protein (e.g., a lysosomal enzyme) wherein deficiency of the protein is associated with a lysosomal storage disorder.

In some embodiments, the therapeutic polypeptide comprises a protein (e.g., an enzyme), wherein deficiency of the protein is associated with a metabolic disorder. In some embodiments, the therapeutic polypeptide comprises a urea cycle enzyme (e.g., ARG1).

In some embodiments, the therapeutic polypeptide comprises a protein (e.g., p53 or PTEN), wherein deficiency of the protein is associated with a cancer. In some embodiments, the therapeutic polypeptide comprises a tumor suppressor.

In some embodiments, the therapeutic polypeptide comprises an amino acid sequence having at least about 80% (e.g., at least about 85%, 90%, 95%, 98%, or more, or 100%) to the amino acid sequence of a wildtype mouse IDUA protein (e.g., SEQ ID NO: 18).

In some embodiments, the therapeutic polypeptide comprises an amino acid sequence having at least about 80% (e.g., at least about 85%, 90%, 95%, 98%, or more, or 100%) to the amino acid sequence of a wildtype human IDUA protein (e.g., SEQ ID NO: 19).

In some embodiments, the therapeutic polypeptide comprises an amino acid sequence having at least about 80% (e.g., at least about 85%, 90%, 95%, 98%, or more, or 100%) to the amino acid sequence of a wildtype mouse OTC protein (e.g., SEQ ID NO: 20).

In some embodiments, the therapeutic polypeptide comprises an amino acid sequence having at least about 80% (e.g., at least about 85%, 90%, 95%, 98%, or more, or 100%) to the amino acid sequence of a wildtype mouse FAH protein (e.g., SEQ ID NO: 21).

In some embodiments, the therapeutic polypeptide comprises an amino acid sequence having at least about 80% (e.g., at least about 85%, 90%, 95%, 98%, or more, or 100%) to the amino acid sequence of a human miniDMD protein (e.g., SEQ ID NO: 22).

In some embodiments, the therapeutic polypeptide comprises an amino acid sequence having at least about 80% (e.g., at least about 85%, 90%, 95%, 98%, or more, or 100%) to the amino acid sequence of a wildtype human DMD protein (e.g., SEQ ID NO: 23).

In some embodiments, the therapeutic polypeptide comprises an amino acid sequence having at least about 80% (e.g., at least about 85%, 90%, 95%, 98%, or more, or 100%) to the amino acid sequence of a wildtype human p53 protein (e.g., SEQ ID NO: 24).

In some embodiments, the therapeutic polypeptide comprises an amino acid sequence having at least about 80% (e.g., at least about 85%, 90%, 95%, 98%, or more, or 100%) to the amino acid sequence of a wildtype human PTEN protein (e.g., SEQ ID NO: 25).

In some embodiments, the therapeutic polypeptide comprises an amino acid sequence having at least about 80% (e.g., at least about 85%, 90%, 95%, 98%, or more, or 100%) to the amino acid sequence of a wildtype human COL3A1 protein (e.g., SEQ ID NO: 56).

In some embodiments, the therapeutic polypeptide comprises an amino acid sequence having at least about 80% (e.g., at least about 85%, 90%, 95%, 98%, or more, or 100%) to the amino acid sequence of a wildtype human BMPR2 protein (e.g., SEQ ID NO: 57).

In some embodiments, the therapeutic polypeptide comprises an amino acid sequence having at least about 80% (e.g., at least about 85%, 90%, 95%, 98%, or more, or 100%) to the amino acid sequence of a wildtype human AHI1 protein (e.g., SEQ ID NO: 58).

In some embodiments, the therapeutic polypeptide comprises an amino acid sequence having at least about 80% (e.g., at least about 85%, 90%, 95%, 98%, or more, or 100%) to the amino acid sequence of a wildtype human FANCC protein (e.g., SEQ ID NO: 59).

In some embodiments, the therapeutic polypeptide comprises an amino acid sequence having at least about 80% (e.g., at least about 85%, 90%, 95%, 98%, or more, or 100%) to the amino acid sequence of a wildtype human MYBPC3 protein (e.g., SEQ ID NO: 60).

In some embodiments, the therapeutic polypeptide comprises an amino acid sequence having at least about 80% (e.g., at least about 85%, 90%, 95%, 98%, or more, or 100%) to the amino acid sequence of a wildtype human ILRG2 protein (e.g., SEQ ID NO: 61).

In some embodiments, the therapeutic polypeptide comprises an amino acid sequence having at least about 80% (e.g., at least about 85%, 90%, 95%, 98%, or more, or 100%) to the amino acid sequence of a wildtype human OTC protein (e.g., SEQ ID NO: 55).

In some embodiments, the therapeutic polypeptide comprises an amino acid sequence having at least about 80% (e.g., at least about 85%, 90%, 95%, 98%, or more, or 100%) to the amino acid sequence of a wildtype human FAH protein (e.g., SEQ ID NO: 54).

v. Peptide Linker

In some embodiments, the various domains in the therapeutic polypeptide (e.g., the various domains of a Spike protein or fragment thereof) may be fused to each other or comprises domains (e.g., an antigenic polypeptide domain and a carrier protein or a multimerization domain) that are fused to each other via a peptide linker. In some embodiments, the antigenic polypeptide a domain of an S protein of a coronavirus fused to a multimerization domain via a peptide linker. Flexible peptide linkers such as glycine linkers, glycine-serine linkers, and linkers containing other amino acids are known in the art (for example, suitable peptide linkers are described by Chen et al. in Fusion Protein Linkers: Property, Design and Functionality. Adv. Drug Deli Rev. 2013 Oct. 15; 65 (10): 1357-1369, incorporated herein by reference in its entirety). Peptide linkers can also be designed by computation methods. The peptide linker can be of any length from 1 to 10, 10 to 20, 20 to 30, 30 to 40, 40 to 50, or greater than 50 amino acids. In some embodiments, the peptide linker comprises the amino acid sequence of SEQ ID NO: 5.

B. Additional circRNA Expression and Circularization Elements

The circRNAs described herein may comprise one or more additional expression elements that facilitate expression and/or circularization of the circRNA.

In some embodiments, the circRNA comprises a Kozak sequence operably linked to a nucleic acid sequence encoding an antigenic polypeptide comprising a Spike(S) protein or a fragment thereof of a coronavirus (e.g., SARS-COV-2). In some embodiments, the Kozak sequence functions as a protein translation initiation site.

In some embodiments, the circRNA comprises a nucleic acid sequence encoding an antigenic polypeptide comprising a Spike(S) protein or a fragment thereof of a coronavirus (e.g., SARS-COV-2), which is operably linked to an internal ribosomal entry site (IRES). In non-limiting examples, the IRES sequence can be a CVB3 virus, EV71 virus, EMCV virus, PV virus, or a CSFV virus IRES sequence. See, for example, Searching for IRES. RNA. 2006 October; 12 (10): 1755-1785, which is incorporated herein by reference in its entirety. In some embodiments, the IRES sequence is a cellular IRES sequence. In some embodiments, the IRES sequence is followed by a Kozak sequence operably linked to the nucleic acid sequence encoding the antigenic polypeptide. In some embodiments, the circRNA further comprises a polyA or polyAC sequence disposed at the 5′ end of the IRES sequence.

In some embodiments, a polyA sequence or polyAC spacer is disposed at the 5′ end of an IRES. In some embodiments, the polyA or polyAC sequence is disposed between the 5′ end of the IRES and the exon-exon splice junction. The internal polyA sequence or polyAC spacer may range from 1 to 500 nucleotides in length (e.g., at least 20, 30, 40, 50, 60, 70, 80, 90, 120, 140, 160, 180, 200, 250, 300, 350, 400, 450, or 500 nucleotides). In some embodiments, the polyA sequence or polyAC sequence may range from 10-70, 20-60, or 30-60 nucleotides in length. In some embodiments, the circRNA comprises the polyAC sequence set forth in SEQ ID NO: 37 disposed at the 5′ end of the IRES sequence. In some embodiments, no polyA sequence or polyAC sequence is disposed at the 5′ end of the IRES sequence. Without being bound by any theory or hypothesis, an internal polyA sequence or a polyAC spacer added before IRES sequences can help to keep the functional second structure of IRES elements for efficient protein translation initiated by IRES. In some embodiments, the polyA sequence or polyAC spacer increases expression of the RNA construct.

In some embodiments, the circRNA comprises a nucleic acid sequence encoding an antigenic polypeptide comprising a Spike(S) protein or a fragment thereof of a coronavirus (e.g., SARS-COV-2), which is operably linked to an m6A (N⁶-methyladenosine) modification motif sequence. The m6A modification sequence can comprise an m6A consensus sequence. M6A consensus sequences are known in the art (for example, consensus sequences identified by Ke et al., 2017 (m⁶A mRNA modifications are deposited in nascent pre-mRNA and are not required for splicing but do specify cytoplasmic turnover. Genes & Dev. 2017. 31:990-1006) and available for download from GEO (GSE86336), the contents of which are incorporated herein by reference in their entireties. In some embodiments, the m6A modification motif sequence comprises the sequence set forth in SEQ ID NO: 38. In some embodiments, the m6A modification motif sequence is followed by a Kozak sequence operably linked to the nucleic acid sequence encoding the antigenic polypeptide.

In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence recognizable by a 3′ catalytic Group I intron fragment comprises the nucleic acid sequence of SEQ ID NO: 39. In some embodiments, the 5′ exon sequence recognizable by a 5′ catalytic Group I intron fragment comprises the nucleic acid sequence of SEQ ID NO: 40. In some embodiments, the 3′ catalytic Group I intron fragment comprises the nucleic acid sequence of SEQ ID NO: 46, and the 5′ catalytic Group I intron fragment sequence comprises the nucleic acid sequence of SEQ ID NO: 47.

In some embodiments, the Group I catalytic intron of the T4 phage Td gene is bisected in such a way to preserve structural elements critical for ribozyme folding. Exon fragment 2 is then ligated upstream of exon fragment 1, and a nucleic acid sequence comprising a sequence encoding the antigenic polypeptide comprising a Spike(S) protein or fragment thereof of a coronavirus is inserted between the exon-exon junction. In some embodiments, the sequence comprising an IRES or m6A sequence, a Kozak sequence, a signal peptide encoding sequence, an antigenic polypeptide comprising an S protein or fragment thereof of a coronavirus, and a stop codon or in-frame 2A peptide sequence is inserted between the exon-exon junction.

In some embodiments, the circRNA comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase).

C. Exemplary Therapeutic circRNAs
i. Exemplary circRNAs for Expression of a Therapeutic Polypeptide

In some embodiments, the circRNA comprises a nucleic acid sequence encoding a therapeutic polypeptide (e.g., any of the therapeutic polypeptides described in Section A above) and further comprising an internal ribosomal entry site (IRES) sequence or an m6A modification motif sequence, wherein the IRES or m6A modification motif sequence is operably linked to the nucleic acid sequence encoding the therapeutic polypeptide. In some embodiments, the IRES sequence is a CVB3 virus, EV71 virus, EMCV virus, PV virus, or a CSFV virus IRES sequence. In some embodiments, the nucleic acid sequence further encodes a signal peptide (SP) fused to the N-terminus of the therapeutic polypeptide (e.g., an antigenic polypeptide, soluble receptor, or antibody). In non-limiting examples, the signal peptide is human tissue plasminogen activator (tPA) or IgE signal peptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide. In some embodiments, the 3′ exon sequence comprises the nucleic acid sequence of SEQ ID NO: 39. In some embodiments, the 5′ exon sequence comprises the nucleic acid sequence of SEQ ID NO: 40. In some embodiments, the 3′ exon sequence comprises the nucleic acid sequence of SEQ ID NO: 39, and the 5′ exon sequence comprises the nucleic acid sequence of SEQ ID NO: 40.

In some embodiments, the circRNA comprises a nucleic acid sequence, the nucleic acid sequence comprising from the 5′ end to the 3′ end: an m6A modification motif sequence, a Kozak sequence, a nucleic acid sequence encoding a therapeutic polypeptide. In some embodiments, the circRNA further comprises an in-frame 2A peptide coding sequence operably linked to the 3′ end of the nucleic acid sequence encoding the therapeutic polypeptide. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide.

In some embodiments, the circRNA comprises a nucleic acid sequence, the nucleic acid sequence comprising from the 5′ end to the 3′ end: an internal ribosomal entry site (IRES) sequence, a Kozak sequence, and a nucleic acid sequence encoding a therapeutic polypeptide. In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide.

In some embodiments, the circRNA comprises a nucleic acid sequence encoding a therapeutic polypeptide, further comprising an in-frame 2A peptide coding sequence operably linked to the 3′ end of the nucleic acid sequence encoding the therapeutic polypeptide (e.g., instead of a stop codon). In some embodiments, the circRNA further comprises an internal ribosomal entry site (IRES) sequence or an m6A modification motif sequence operably linked to the nucleic acid sequence encoding the therapeutic polypeptide. In some embodiments, the nucleic acid sequence further encodes a SP (e.g., human tPA or IgE SP) fused to the N-terminus of the therapeutic polypeptide for secretion of the therapeutic polypeptide.

In some embodiments, the circRNA comprises a nucleic acid sequence, the nucleic acid sequence comprising from the 5′ end to the 3′ end: an internal ribosomal entry site (IRES) sequence or an m6A modification motif sequence, a Kozak sequence, a nucleic acid sequence encoding a therapeutic polypeptide, and an in-frame 2A peptide coding sequence. In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the therapeutic polypeptide, and a 5′ exon sequence recognizable by a 5′ catalytic Group I intron fragment flanking the 3′ end of the nucleic acid sequence encoding the therapeutic polypeptide.

ii. Exemplary circRNA Vaccines

In some embodiments, the method comprises administration of a circRNA vaccine (such as any of the circRNA vaccines described herein, or any vaccine comprising the naked circRNA described herein). In some embodiments, the circRNA vaccine comprises a naked circRNA comprising a nucleic acid sequence encoding an antigenic polypeptide comprising a Spike(S) protein or a fragment thereof of a coronavirus (e.g., SARS-COV2), and further comprising an internal ribosomal entry site (IRES) sequence or an m6A modification motif sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof. In non-limiting examples, the signal peptide is human tissue plasminogen activator (tPA) or IgE signal peptide. In some embodiments, the circRNA further comprises a polyA or polyAC sequence disposed at the 5′ end of an IRES sequence.

In some embodiments, the circRNA vaccine comprises a naked circRNA comprising a nucleic acid sequence encoding an antigenic polypeptide comprising a Spike(S) protein or a fragment thereof of a coronavirus (e.g., SARS-COV-2), and an internal ribosomal entry site (IRES) sequence or an m6A modification motif sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof. In non-limiting examples, the signal peptide is human tissue plasminogen activator (tPA) or IgE signal peptide. In some embodiments, the circRNA further comprises a polyA or polyAC sequence disposed at the 5′ end of an IRES sequence.

In some embodiments, the circRNA vaccine comprises a naked circRNA comprising a nucleic acid sequence encoding an antigenic polypeptide comprising a Spike(S) protein or a fragment thereof of a coronavirus (e.g., SARS-COV-2), and further comprising an internal ribosomal entry site (IRES) or m6A modification motif sequence, and a Kozak sequence operably linked to the nucleic acid sequence encoding the antigenic polypeptide. In some embodiments, the nucleic acid sequence further encodes a signal peptide (SP) fused to the N-terminus of the S protein or fragment thereof. In some embodiments, the signal peptide is, for example, human tissue plasminogen activator (tPA) or IgE signal peptide. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence comprises the nucleic acid sequence of SEQ ID NO: 39. In some embodiments, the 5′ exon sequence comprises the nucleic acid sequence of SEQ ID NO: 40. In some embodiments, the 3′ exon sequence comprises the nucleic acid sequence of SEQ ID NO: 39, and the 5′ exon sequence comprises the nucleic acid sequence of SEQ ID NO: 40.

In some embodiments, the circRNA vaccine comprises a naked circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), and a nucleic acid sequence encoding an antigenic polypeptide comprising a Spike(S) protein or a fragment thereof of a coronavirus (e.g., SARS-COV-2). In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a naked circRNA comprising a nucleic acid sequence, the nucleic acid sequence comprising from the 5′ end to the 3′ end: an m6A modification motif sequence, a Kozak sequence, a nucleic acid sequence encoding a signal peptide (SP), and a nucleic acid sequence encoding an antigenic polypeptide comprising a Spike(S) protein or a fragment thereof of a coronavirus (e.g., SARS-COV-2). In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a naked circRNA comprising a nucleic acid sequence encoding an antigenic polypeptide comprising a receptor-binding domain (RBD) of a Spike(S) protein of a coronavirus (e.g., SARS-COV-2) and a multimerization domain (e.g., C-terminal domain of a T4 fibritin protein that mediates trimerization of the T4 fibritin protein). In some embodiments, the circRNA further comprises an internal ribosomal entry site (IRES) sequence or an m6A modification sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof (e.g., human tPA or IgE signal peptide). In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a naked circRNA comprising a nucleic acid sequence encoding an antigenic polypeptide comprising a receptor-binding domain (RBD) of a Spike(S) protein of a coronavirus (e.g., derived from the wild type or B.1.351/501Y.V2/BA.5/BF.7 (sub-)variant of SARS-COV-2) and a multimerization domain (e.g., C-terminal domain of a T4 fibritin protein that mediates trimerization of the T4 fibritin protein). In some embodiments, the circRNA further comprises an internal ribosomal entry site (IRES) sequence or an m6A modification sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof (e.g., human tPA or IgE signal peptide). In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments the RBD domain has at least 90% (e.g., at least 95%, 96%, 97%, 98%, 99%) sequence identity to the amino acid sequence of SEQ ID NO: 2. In some embodiments, the RBD domain comprises the amino acid sequence of SEQ ID NO: 2. In some embodiments the RBD domain has at least 90% (e.g., at least 95%, 96%, 97%, 98%, 99%) sequence identity to the amino acid sequence of SEQ ID NO: 63. In some embodiments, the RBD domain comprises the amino acid sequence of SEQ ID NO: 63.

In some embodiments, the circRNA vaccine comprises a circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), and a nucleic acid sequence encoding an antigenic polypeptide comprising a receptor-binding domain (RBD) of a Spike(S) protein of a coronavirus (e.g., derived from the wild type SARS-COV-2 or a variant such as the B.1.351, B.1.617.2, BA.5 or BF.7 (sub-)variant of SARS-COV-2). In some embodiments, the IRES sequence is a CVB3 virus, EV71 virus, EMCV virus, PV virus, or a CSFV virus IRES sequence. In some embodiments, the circRNA further comprises a polyAC sequence disposed at the 5′ end of the IRES sequence. In some embodiments, the circRNA further comprises the polyAC sequence set forth in SEQ ID NO: 37 disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence comprises the nucleic acid sequence of SEQ ID NO: 39. In some embodiments, the 5′ exon sequence comprises the nucleic acid sequence of SEQ ID NO: 40. In some embodiments, the 3′ exon comprises the nucleic acid sequence of SEQ ID NO: 39, and the 5′ exon sequence comprises the nucleic acid sequence of SEQ ID NO: 40.

In some embodiments, the circRNA vaccine comprises a circRNA comprising a nucleic acid sequence, the nucleic acid sequence comprising from the 5′ end to the 3′ end: an m6A modification motif sequence, a Kozak sequence, a nucleic acid sequence encoding a signal peptide (SP), and a nucleic acid sequence encoding an antigenic polypeptide comprising a receptor-binding domain (RBD) of a Spike(S) protein of a coronavirus (e.g., derived from the wild type SARS-CoV-2 or a variant such as the B.1.351, B.1.617.2, BA.5 or BF.7 (sub-)variant of SARS-COV-2). In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence comprises the nucleic acid sequence of SEQ ID NO: 39. In some embodiments, the 5′ exon sequence comprises the nucleic acid sequence of SEQ ID NO: 40. In some embodiments, the 3′ exon comprises the nucleic acid sequence of SEQ ID NO: 39, and the 5′ exon sequence comprises the nucleic acid sequence of SEQ ID NO: 40.

In some embodiments, the circRNA vaccine comprises a circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), and a nucleic acid sequence encoding an antigenic polypeptide comprising a receptor-binding domain (RBD) of a Spike(S) protein of a coronavirus (e.g., derived from the wild type SARS-COV-2 or a variant such as the B.1.351, B.1.617.2, BA.5 or BF.7 (sub-)variant of SARS-COV-2) and a multimerization domain (e.g., C-terminal domain of a T4 fibritin protein that mediates trimerization of the T4 fibritin protein). In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments the RBD domain has at least 90% (e.g., at least 95%, 96%, 97%, 98%, 99%) sequence identity to the amino acid sequence of SEQ ID NO: 2. In some embodiments, the RBD domain comprises the amino acid sequence of SEQ ID NO: 2. In some embodiments the RBD domain has at least 90% (e.g., at least 95%, 96%, 97%, 98%, 99%) sequence identity to the amino acid sequence of SEQ ID NO: 63. In some embodiments, the RBD domain comprises the amino acid sequence of SEQ ID NO: 63.

In some embodiments, the circRNA vaccine comprises a circRNA comprising a nucleic acid sequence encoding an antigenic polypeptide comprising a receptor-binding domain (RBD) and an S2 region of a Spike(S) protein of a coronavirus (e.g., SARS-COV-2) and a multimerization domain (e.g., C-terminal domain of a T4 fibritin protein that mediates trimerization of the T4 fibritin protein). In some embodiments, the circRNA further comprises an internal ribosomal entry site (IRES) or m6A modification motif sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof (e.g., human tPA or IgE signal peptide). In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), and a nucleic acid sequence encoding an antigenic polypeptide comprising a receptor-binding domain (RBD) and an S2 region of a Spike(S) protein of a coronavirus (e.g., derived from the wild type SARS-COV-2 or a variant such as the B.1.351, B.1.617.2, BA.5 or BF.7 (sub-)variant of SARS-COV-2) and a multimerization domain (e.g., C-terminal domain of a T4 fibritin protein that mediates trimerization of the T4 fibritin protein). In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence, the nucleic acid sequence comprising from the 5′ end to the 3′ end: a m6A modification motif sequence, a Kozak sequence, a nucleic acid sequence encoding a signal peptide (SP), and a nucleic acid sequence encoding an antigenic polypeptide comprising a receptor-binding domain (RBD) and an S2 region of a Spike(S) protein of a coronavirus (e.g., derived from the wild type SARS-COV-2 or a variant such as the B.1.351, B.1.617.2, BA.5 or BF.7 (sub-)variant of SARS-COV-2) and a multimerization domain (e.g., C-terminal domain of a T4 fibritin protein that mediates trimerization of the T4 fibritin protein). In some embodiments, the IRES sequence is a CVB3 virus, EV71 virus, EMCV virus, PV virus, or a CSFV virus IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide, the antigenic polypeptide comprising amino acid residues 2-1273 of a full-length S protein of SARS-COV-2, wherein the numbering is based on SEQ ID NO: 1. In some embodiments, the circRNA further comprises an internal ribosomal entry site (IRES) or m6A modification motif sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide, the antigenic polypeptide comprising amino acid residues 2-1273 of a full-length S protein of SARS-COV-2, wherein the numbering is based on SEQ ID NO: 1. In some embodiments, the circRNA further comprises an internal ribosomal entry site (IRES) or m6A modification motif sequence, and a Kozak sequence operably linked to the nucleic acid sequence encoding the antigenic polypeptide. In some embodiments, the nucleic acid sequence further encodes a signal peptide (SP) fused to the N-terminus of the S protein or fragment thereof. In some embodiments, the signal peptide is, for example, human tissue plasminogen activator (tPA) or IgE signal peptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), and a nucleic acid sequence encoding an antigenic polypeptide, the antigenic polypeptide comprising amino acid residues 2-1273 of a full-length S protein of SARS-COV-2, wherein the numbering is based on SEQ ID NO: 1. In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence, the nucleic acid sequence comprising from the 5′ end to the 3′ end: a m6A modification motif sequence, a Kozak sequence, a nucleic acid sequence encoding a signal peptide (SP), and a nucleic acid sequence encoding an antigenic polypeptide, the antigenic polypeptide comprising amino acid residues 2-1273 of a full-length S protein of SARS-COV-2, wherein the numbering is based on SEQ ID NO: 1. In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a Spike(S) protein or a fragment thereof of SARS-COV-2 wherein the antigenic polypeptide comprises an S2 region of the S protein. In some embodiments, the S2 region comprises amino acid residues 686 to 1273 of a full-length S protein of SARS-COV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises K986P and V987P mutations. In some embodiments, the S2 region comprises the amino acid sequence of SEQ ID NO: 7. In some embodiments, the circRNA further comprises an internal ribosomal entry site (IRES) or m6A modification motif sequence, wherein the IRES or m6A modification motif sequence is operably linked to the nucleic acid sequence encoding the antigenic polypeptide. In some embodiments, the nucleic acid sequence further encodes a signal peptide (SP) fused to the N-terminus of the S protein or fragment thereof (e.g., human tPA or IgE signal peptide). In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the present application provides a circular RNA (circRNA) vaccine comprising a circRNA comprising a nucleic acid sequence set forth in any one of SEQ ID NOs: 11-15 or SEQ ID NOs: 48-49.

In some embodiments, the circRNA vaccine comprises a circRNA comprising a nucleic acid sequence encoding an antigenic polypeptide comprising a Spike(S) protein or a fragment thereof of a coronavirus (e.g., SARS-COV-2), further comprising an in-frame 2A peptide coding sequence operably linked to the 3′ end of the nucleic acid sequence encoding the antigenic polypeptide. In some embodiments, the circRNA further comprises an internal ribosomal entry site (IRES) sequence operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof.

In some embodiments, the circRNA vaccine comprises a circRNA comprising a nucleic acid sequence, the nucleic acid sequence comprising from the 5′ end to the 3′ end: an internal ribosomal entry site (IRES) sequence, a Kozak sequence, a nucleic acid sequence encoding a signal peptide (SP), a nucleic acid sequence encoding an antigenic polypeptide comprising an S protein or fragment thereof of a coronavirus, and an in-frame 2A peptide coding sequence. In some embodiments, the IRES sequence is a CVB3 virus, EV71 virus, EMCV virus, PV virus, or a CSFV virus IRES sequence. In some embodiments, the circRNA further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence, the nucleic acid sequence comprising from the 5′ end to the 3′ end: an m6A modification motif sequence, a Kozak sequence, a nucleic acid sequence encoding a signal peptide (SP), a nucleic acid sequence encoding an antigenic polypeptide comprising an S protein or fragment thereof of a coronavirus, and an in-frame 2A peptide coding sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a Spike(S) protein or a fragment thereof of a coronavirus (e.g., derived from the wild type SARS-COV-2 or a variant such as the B.1.351,B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the circRNA further comprises an m6A modification motif sequence operably linked to the nucleic acid sequence encoding the antigenic polypeptide. In some embodiments, the nucleic acid sequence further comprises a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a Spike(S) protein or a fragment thereof of a coronavirus (e.g., derived from the wild type SARS-COV-2 or a variant such as the B.1.351, B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof, and a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the 3′ exon sequence comprises the nucleic acid sequence of SEQ ID NO: 39. In some embodiments, the 5′ exon sequence comprises the nucleic acid sequence of SEQ ID NO: 40. In some embodiments, the 3′ exon sequence comprises the nucleic acid sequence of SEQ ID NO: 39, and the 5′ exon sequence comprises the nucleic acid sequence of SEQ ID NO: 40.

In some embodiments, the circRNA vaccine comprises a circRNA comprising a nucleic acid sequence encoding an antigenic polypeptide comprising a receptor-binding domain (RBD) of a Spike(S) protein of a coronavirus (e.g., derived from the wild type SARS-COV-2 or a variant such as the B.1.351, B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the circRNA further comprises an internal ribosomal entry site (IRES) or m6A modification motif sequence, wherein the IRES or m6A modification motif sequence is operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) of a Spike(S) protein of a coronavirus (e.g., derived from the wild type SARS-COV-2 or a variant such as the B.1.351, B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) or m6A modification motif sequence, wherein the IRES or m6A modification motif sequence is operably linked to the nucleic acid sequence encoding the antigenic polypeptide. In some embodiments, the nucleic acid sequence further comprises a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) and an S2 region of a Spike(S) protein of a coronavirus (e.g., derived from the wild type SARS-CoV-2 or a variant such as the B.1.351, B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the antigenic polypeptide further comprises a multimerization domain (e.g., a C-terminal Foldon domain of a T4 fibritin protein or a GCN4-based isoleucine zipper domain). In some embodiments, the circRNA further comprises an internal ribosomal entry site (IRES) sequence (e.g., a CVB3 virus, EV71 virus, EMCV virus, PV virus, or CSFV virus IRES sequence) or an m6A modification motif sequence, wherein the IRES or m6A modification motif sequence is operably linked to the nucleic acid sequence encoding the antigenic polypeptide. In some embodiments, the nucleic acid sequence further comprises a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) and an S2 region of a Spike(S) protein of a coronavirus (e.g., derived from the wild type SARS-CoV-2 or a variant such as the B.1.351, B.1.617.2, BA.5 or BF.7 (sub-)variant of SARS-COV-2), further comprising an in-frame 2A peptide coding sequence operably linked to the 3′ end of the nucleic acid sequence encoding the antigenic polypeptide. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof, and a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments the RBD domain has at least 90% (e.g., at least 95%, 96%, 97%, 98%, 99%) sequence identity to the amino acid sequence of SEQ ID NO: 2. In some embodiments, the RBD domain comprises the amino acid sequence of SEQ ID NO: 2. In some embodiments the RBD domain has at least 90% (e.g., at least 95%, 96%, 97%, 98%, 99%) sequence identity to the amino acid sequence of SEQ ID NO: 63. In some embodiments, the RBD domain comprises the amino acid sequence of SEQ ID NO: 63.

In some embodiments, the circRNA vaccine comprises a circRNA comprising a nucleic acid sequence encoding amino acid residues 2-1273 of a full-length S protein of SARS-COV-2, wherein the numbering is based on SEQ ID NO: 1, further comprising an in-frame 2A peptide coding sequence operably linked to the 3′ end of the nucleic acid sequence encoding the antigenic polypeptide. In some embodiments, the antigenic polypeptide further comprises a multimerization domain. In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further comprises a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding amino acid residues 2-1273 of a full-length S protein of SARS-COV-2, wherein the numbering is based on SEQ ID NO: 1, further comprising an in-frame 2A peptide coding sequence operably linked to the 3′ end of the nucleic acid sequence encoding the antigenic polypeptide. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding amino acid residues 2-1273 of a full-length S protein of SARS-COV-2, wherein the numbering is based on SEQ ID NO: 1, further comprising an in-frame 2A peptide coding sequence operably linked to the 3′ end of the nucleic acid sequence encoding the antigenic polypeptide. In some embodiments, the antigenic polypeptide further comprises a multimerization domain. In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof, and a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) and an S2 region of a Spike (S) protein of a coronavirus (e.g., SARS-COV-2), further comprising an in-frame 2A peptide coding sequence operably linked to the 3′ end of the nucleic acid sequence encoding the antigenic polypeptide. In some embodiments, the S2 region comprises amino acid residues 686 to 1273 of a full-length S protein of SARS-COV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises the amino acid sequence of SEQ ID NO: 7. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further comprises a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) and an S2 region of a Spike(S) protein of a coronavirus (e.g., SARS-COV-2), further comprising an in-frame 2A peptide coding sequence operably linked to the 3′ end of the nucleic acid sequence encoding the antigenic polypeptide. In some embodiments, the S2 region comprises amino acid residues 686 to 1273 of a full-length S protein of SARS-COV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises the amino acid sequence of SEQ ID NO: 7. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine comprises a circRNA comprising a nucleic acid sequence encoding an antigenic polypeptide comprising a receptor-binding domain (RBD) and an S2 region of a Spike(S) protein of a coronavirus (e.g., SARS-COV-2), further comprising an in-frame 2A peptide coding sequence operably linked to the 3′ end of the nucleic acid sequence encoding the antigenic polypeptide. In some embodiments, the S2 region comprises amino acid residues 686 to 1273 of a full-length S protein of SARS-COV-2, wherein the numbering is based on SEQ ID NO: 1. In some embodiments, the S2 region comprises the amino acid sequence of SEQ ID NO: 6. In some embodiments, the S2 region comprises one or more mutations that stabilize a pre-fusion conformation of the S protein. In some embodiments, the S2 region comprises the amino acid sequence of SEQ ID NO: 7. In some embodiments, the antigenic polypeptide further comprises a multimerization domain (e.g., C-terminal domain of a T4 fibritin protein that mediates trimerization of the T4 fibritin protein). In some embodiments, the circRNA further comprises an internal ribosomal entry site (IRES) sequence (e.g., a CVB3 virus, EV71 virus, EMCV virus, PV virus, or CSFV virus IRES sequence) operably linked to the nucleic acid sequence encoding the antigenic polypeptide. In some embodiments, the nucleic acid sequence further encodes a SP (e.g., human tPA or IgE SP) fused to the N-terminus of the S protein or fragment thereof, and a Kozak sequence operably linked to the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine provided comprises a nucleic acid sequence comprising from the 5′ end to the 3′ end: the IRES sequence, the Kozak sequence, the SP, the nucleic acid sequence encoding the antigenic polypeptide, and the in-frame 2A peptide coding sequence. In some embodiments, the circRNA vaccine further comprises a polyA or polyAC sequence disposed at the 5′ end of the IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA vaccine provided comprises a nucleic acid sequence comprising from the 5′ end to the 3′ end: the polyA or polyAC sequence, the IRES sequence, the Kozak sequence, the SP, the nucleic acid sequence encoding the antigenic polypeptide, and the in-frame 2A peptide coding sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide.

In some embodiments, the circRNA comprises a nucleic acid sequence comprising from the 5′ end to the 3′ end: the m6A modification motif sequence, the Kozak sequence, the SP, the nucleic acid sequence encoding antigenic polypeptide, and the in-frame 2A peptide coding sequence. In some embodiments, the antigenic polypeptide comprises a receptor-binding domain (RBD) of the S protein. In some embodiments, the antigenic polypeptide further comprises a multimerization domain (e.g., C-terminal domain of a T4 fibritin protein that mediates trimerization of the T4 fibritin protein).

In some embodiments, the circRNA comprises a nucleic acid sequence comprising from the 5′ end to the 3′ end: the m6A modification motif sequence, the Kozak sequence, the SP, and the nucleic acid sequence encoding the antigenic polypeptide. In some embodiments, the antigenic polypeptide comprises a receptor-binding domain (RBD) and an S2 region of the S protein. In some embodiments, the antigenic polypeptide further comprises a multimerization domain (e.g., C-terminal domain of a T4 fibritin protein that mediates trimerization of the T4 fibritin protein).

In some embodiments, the circRNA comprises a nucleic acid sequence comprising from the 5′ end to the 3′ end: the m6A modification motif sequence, the Kozak sequence, the SP, and a sequence encoding the antigenic polypeptide. In some embodiments, the antigenic polypeptide comprises amino acid residues 2-1273 of a full-length S protein of SARS-COV-2, wherein the numbering is based on SEQ ID NO: 1. In some embodiments, the antigenic polypeptide further comprises a multimerization domain (e.g., C-terminal domain of a T4 fibritin protein that mediates trimerization of the T4 fibritin protein). In some embodiments, the circRNA comprises an in-frame 2A peptide coding sequence following the antigenic polypeptide.

### SARS-Cov-2 Variants

The antigenic polypeptides described herein can be derived from a SARS-COV-2 variant. Exemplary SARS-COV-2 variants and spike protein mutations associated with these variants are shown in Table A below. The circRNA vaccines and compositions described herein may be used for treating any one of the SARS-COV-2 variants described herein or combinations thereof. The SARS-COV-2 variants described herein are named by the World Health Organization or according to the Phylogenetic Assignment of Named Global Outbreak (PANGO) Lineages software. It is understood that the same variants may be referred to using different naming systems and algorithms in the art. SARS-COV-2 variant classifications and definitions, as well as a list of known SARS-COV-2 variants can be found at world wide web.cdc.gov/coronavirus/2019-ncov/variants/variant-classifications.html.

TABLE A
SARS-CoV-2 variants

| WHO label | Pango Lineage | Type | S protein mutation(s) |
|---|---|---|---|
| Alpha | B.1.1.7 and Q | Variant being | 69del, 70del, 144del, (E484K*), (S494P*), |
| | lineages | monitored | N501Y, A570D, D614G, P681H, T716I, S982A, |
| | | (VBM) | D1118H (K1191N*) |
| Beta | B.1.351 and | VBM | D80A, D215G, 241del, 242del, 243del, K417N, |
| | descendent | | E484K, N501Y, D614G, A701V |
| | lineages | | |
| Gamma | P.1and descendent | VBM | L18F, T20N, P26S, D138Y, R190S, K417T, |
| | lineages | | E484K, N501Y, D614G, H655Y, T1027I |
| Epsilon | B.1.427 | VBM | L452R, D614G |
| | B.1.429 | | S13I, W152C |
| Eta | B.1.525 | VBM | A67V, 69del, 70del, 144del, E484K, D614G, |
| | | | Q677H, F888L |
| Iota | B.1.526 | VBM | (L5F*), T95I, D253G, (S477N*), (E484K*), |
| | | | D614G, (A701V*) |
| Kappa | B.1.617.1 | VBM | (T95I), G142D, E154K, L452R, E484Q, D614G, |
| | | | P681R, Q1071H |
| N/A | B.1.617.3 | VBM | T19R, G142D, L452R, E484Q, D614G, P681R, |
| | | | D950N |
| Zeta | P.2 | VBM | E484K, (F565L*), D614G, V1176F |
| Mu | B.1.621, B.1.621.1 | VBM | D80G, 144del, F157S, L452R, D614G, (T791I*), |
| | | | (T859N*), D950H |
| Delta | B.1.617.2 and | Variant of | T19R, (V70F*), T95I, G142D, E156-, F157-, |
| | AY lineages | Concern | R158G, (A222V*), (W258L*), (K417N*), L452R, |
| | | (VOC) | T478K, D614G, P681R, D950N |
| Omicron | B.1.1.529 and BA | VOC | A67V, del69-70, T95I, del142-144, Y145D, |
| | lineages | | del211, L212I, ins214EPE, G339D, S371L, |
| | | | S373P, S375F, K417N, N440K, G446S, S477N, |
| | | | T478K, E484A, Q493R, G496S, Q498R, N501Y, |
| | | | Y505H, T547K, D614G, H655Y, N679K, P681H, |
| | | | N764K, D796Y, N856K, Q954H, N969K, L981F |

The reference SARS-COV-2 virus is BetaCoV/WIV04/2019 (Accession ID: EPI-ISL-402124). In some embodiments, the SARS-COV-2 variant is a variant being monitored, a variant of interest, a variant of concern, or a variant of high consequence. In some embodiments, the SARS-COV-2 variant is selected from the group consisting of an Alpha (i.e., B.1.1.7 and Q) variant, a Beta (i.e., B.1.351) variant, a Gamma (i.e., P.1, also known as B.1.1.28.1) variant, an Epsilon (i.e., B.1.427 or B.1.429) variant, an Eta (i.e., B.1.525) variant, an Iota (i.e., B.1.526) variant, a Kappa (i.e., B.1.617.1) variant, a B. 1.617.3 variant, a Zeta (i.e., P.2) variant, a Mu (i.e., B.1.621 or B.1.621.1) variant, a Delta (i.e., B.1.617.2 or AY) variant, and an Omicron (i.e., B.1.1.529 or BA) variant. In some embodiments, the SARS-COV-2 variant is a Delta variant, such as a B.1.617.2 variant, or an AY variant. In some embodiments, the SARS-COV-2 variant is an Omicron variant, such as a B.1.529 variant or a BA variant or sub-variants thereof. In some embodiments, the SARS-COV-2 variant is a BA variant of an Omicron variant (e.g. BA.1, BA.1.1, BA.2, BA.3, BA.4, or BA.5). In some embodiments, the SARS-COV-2 variant is a sublineage of omicron sub-variants (e.g. BA.2.75.2, BA.4.6, BF.7 or BQ.1.1). In some embodiments, the SARS-CoV-2 variant has one or more mutations (e.g., insertion, deletion, and/or substitution) in the spike protein. In some embodiments, the one or more mutations in the spike protein may affect viral fitness, such as transmissibility, virulence, and/or drug resistance (e.g., resistance to neutralizing antibodies and/or resistance to a vaccine). In some embodiments, the one or more mutations in the spike protein do not substantially alter viral fitness. In some embodiments, the SARS-COV-2 variant does not have a mutation in the spike protein.

### IV. Methods of Treating or Preventing a Disease or Condition

The circRNAs and compositions derived herein may be used to treat or prevent a disease or condition in an individual, including, but not limited to genetic diseases (e.g., hereditary genetic diseases, metabolic diseases and cancer), and infections (e.g., viral infections such as coronavirus infections). In some embodiments, the circRNA is subject to rolling circle translation by a ribosome in the individual.

In some embodiments, there is provided a method of treating or preventing a disease or condition in an individual, comprising administering to the individual an effective amount of a circRNA comprising a nucleic acid sequence encoding an antigenic polypeptide, wherein the circRNA is a naked circRNA. In some embodiments, the circRNA is administered two or more times. In some embodiments, the interval between each administration is at least about four weeks, such as at least any of five, six, seven, or eight weeks. In some embodiments, the antigenic polypeptide is a protein or a fragment thereof of an infectious agent, such as a virus, e.g., a coronavirus. In some embodiments, the infectious agent is SARS-COV-2. In some embodiments, the antigenic polypeptide is an S protein or fragment thereof. In some embodiments, the disease or condition is a coronavirus infection. In some embodiments, the method comprises administering an effective amount of a cocktail composition comprising a plurality of circRNA encoding different antigenic polypeptides.

In some embodiments, there is provided a method of treating or preventing a disease or condition in an individual, comprising administering to the individual an effective amount of a circRNA comprising a nucleic acid sequence encoding a functional protein, wherein the circRNA is a naked RNA. In some embodiments, the circRNA is administered two or more times. In some embodiments, the interval between each administration is at least about four weeks, such as at least any of five, six, seven, or eight weeks. In some embodiments, the functional protein is an enzyme, a receptor, a ligand, a signaling molecule, or a transcription factor. In some embodiments, the disease or condition is a metabolic disease. In some embodiments, the disease or condition is a lysosomal storage disorder. In some embodiments, the disease or condition is a cancer.

In some embodiments, there is provided a method of treating or preventing a disease or condition in an individual, comprising administering to the individual an effective amount of a circRNA comprising a nucleic acid sequence encoding a receptor protein, wherein the circRNA is a naked circRNA. In some embodiments, the circRNA is administered two or more times. In some embodiments, the interval between each administration is at least about four weeks, such as at least any of five, six, seven, or eight weeks. In some embodiments, the receptor protein is a receptor of an infectious agent, such as a virus, e.g., a coronavirus. In some embodiments, the receptor protein is a soluble receptor, such as a soluble ACE2 receptor.

In some embodiments, there is provided a method of treating or preventing a disease or condition in an individual, comprising administering to the individual an effective amount of a circRNA comprising a nucleic acid sequence encoding a targeting protein, such as an antibody, wherein the circRNA is a naked circRNA. In some embodiments, the circRNA is administered two or more times. In some embodiments, the interval between each administration is at least about four weeks, such as at least any of five, six, seven, or eight weeks. In some embodiments, the targeting protein is a neutralizing antibody. In some embodiments, the targeting protein is a therapeutic antibody. In some embodiments, the targeting protein specifically binds an infectious agent, such as a virus, e.g., a coronavirus.

In some embodiments, the present application provides naked circRNAs for use in treating or preventing a disease or condition in an individual.

In some embodiments, the present application provides naked circRNA vaccines for use in treating or preventing a coronavirus (e.g., SARS-COV, MERS-COV, or SARS-COV-2) infection in an individual.

In some embodiments, the present application provides use of a naked circRNA comprising a nucleic acid sequence encoding a therapeutic polypeptide for the manufacture of a medicament for treating or preventing a disease or condition in an individual.

In some embodiments, the present application provides use of a circRNA vaccine comprising a naked circRNA comprising a nucleic acid sequence encoding an antigenic polypeptide comprising a Spike(S) protein or a fragment thereof of a coronavirus (e.g., SARS-CoV-2) for the manufacture of a vaccine for treating or preventing a coronavirus infection in an individual.

### A. Treating a Genetic Disease or Condition

The circRNAs described herein may be used for treating a genetic disease or condition that is associated with a mutation or deficiency in a naturally-occurring protein corresponding to the therapeutic polypeptide encoded by the circRNA. In some embodiments, the disease or condition is a disease or condition associated with insufficient levels and/or activity of a naturally-occurring protein corresponding to the therapeutic polypeptide. In some embodiments, the disease or condition is a hereditary genetic disease associated with one or more mutations in naturally-occurring protein corresponding to the therapeutic polypeptide. In some embodiments, the therapeutic polypeptide is a wildtype protein, or a functional variant thereof (e.g., a functional fragment, fusion protein, or mutant).

In some aspects, the present application provides methods and compositions for treatment of a disease or condition associated with a deficiency of a functional protein, such as an enzyme (e.g., IDUA) using a circRNA expressing a therapeutic polypeptide, wherein the circRNA is a naked circRNA. In some embodiments, the therapeutic polypeptide comprises a nucleotide sequence encoding the protein or a derivative thereof. In some embodiments, the circRNA is capable of expressing a functional protein or functional derivative of a protein that is capable of restoring function of the protein associated with the disease or condition. In some embodiments, the circRNA is capable of restoring 10%, 20%, 30%, 40%, 50%, 60%, 70%, 80%, 90%, or 100% of the activity of the protein as compared to the endogenous wild-type protein in a cell or organism (e.g., a mouse or a human), e.g., up to 8, 12, 16, 24, 30, 36, or 40 hours after administration of the circRNA.

In some embodiments, the therapeutic polypeptide can be any polypeptide that is capable of being expressed by target cells (e.g., human or mouse cells) for the production (and in certain instances, the excretion) of a functional enzyme or protein as disclosed, for example, in International Application No. PCT/US2010/058457, incorporated herein by reference in its entirety. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

Examples of disease-associated mutations that may be treated by the methods of the present application include, but are not limited to, TP53^{W53X} (e.g., 158G>A) associated with cancer, IDUA^{W402X} (e.g., TGG>TAG mutation in exon 9) associated with Mucopolysaccharidosis type I (MPS I), COL3A1^{W1278X} (e.g., 3833G>A mutation) associated with Ehlers-Danlos syndrome, BMPR2^{W298X} (e.g., 893G>A) associated with primary pulmonary hypertension, AHI1^{W725X} (e.g., 2174G>A) associated with Joubert syndrome, FANCC^{W506X} (e.g., 1517G>A) associated with Fanconi anemia, MYBPC3^{W1098X} (e.g., 3293G>A) associated with primary familial hypertrophic cardiomyopathy, and IL2RG^{W237X} (e.g., 710G>A) associated with X-linked severe combined immunodeficiency. In some embodiments, the disease or condition is a cancer. In some embodiments, the disease or condition is a monogenetic disease. In some embodiments, the disease or condition is a polygenetic disease.

In some embodiments, the disease or condition is a liver disease or condition. In some embodiments, the disease or condition is a disease or condition of the respiratory tract of the individual, such as a lung disease or condition.

In some embodiments, there is provided a method of treating a cancer in an individual, comprising administering to the individual an effective amount of a circRNA comprising a nucleic acid sequence encoding a tumor suppressor, wherein the circRNA is a naked circRNA. In some embodiments, the tumor suppressor is TP53 (including a functional variant thereof). In some embodiments, the tumor suppressor is PTEN (including a functional variant thereof). In some embodiments, the tumor suppressor comprises an amino acid sequence having at least about 80% (e.g., at least about any one of 85%, 90%, 93%, 95%, 97%, 98%, or 99%, or 100%) to the amino acid sequence of SEQ ID NO: 24 or 25.

In some embodiments, there is provided a method of treating a lysosomal storage disorder in an individual, comprising administering to the individual an effective amount of a circRNA comprising a nucleic acid sequence encoding a lysosomal enzyme, wherein the circRNA is a naked circRNA.

In some embodiments, there is provided a method of treating a liver disease or condition in an individual, comprising administering to the individual an effective amount of a circRNA comprising a nucleic acid sequence encoding a liver protein (e.g., an enzyme), wherein the circRNA is a naked circRNA.

In some embodiments, there is provided a method of treating Mucopolysaccharidosis type I (MPS I) in an individual, comprising administering to the individual an effective amount of a circRNA comprising a nucleic acid sequence encoding IDUA (including a functional variant thereof), wherein the circRNA is a naked circRNA. In some embodiments, the IDUA comprises an amino acid sequence having at least about 80% (e.g., at least about any one of 85%, 90%, 93%, 95%, 97%, 98%, or 99%, or 100%) to the amino acid sequence of SEQ ID NO: 18. In some embodiments, the IDUA comprises an amino acid sequence having at least about 80% (e.g., at least about any one of 85%, 90%, 93%, 95%, 97%, 98%, or 99%, or 100%) to the amino acid sequence of SEQ ID NO: 19.

In some embodiments, there is provided a method of treating ornithine transcarbamylase deficiency in an individual, comprising administering to the individual an effective amount of a circRNA comprising a nucleic acid sequence encoding OTC (including a functional variant thereof), wherein the circRNA is a naked circRNA. In some embodiments, the OTC comprises an amino acid sequence having at least about 80% (e.g., at least about any one of 85%, 90%, 93%, 95%, 97%, 98%, or 99%, or 100%) to the amino acid sequence of SEQ ID NO: 20. In some embodiments, the OTC comprises an amino acid sequence having at least about 80% (e.g., at least about any one of 85%, 90%, 93%, 95%, 97%, 98%, or 99%, or 100%) to the amino acid sequence of SEQ ID NO: 56.

In some embodiments, there is provided a method of treating tyrosinemia in an individual, comprising administering to the individual an effective amount of a circRNA comprising a nucleic acid sequence encoding FAH (including a functional variant thereof), wherein the circRNA is a naked circRNA. In some embodiments, the FAH comprises an amino acid sequence having at least about 80% (e.g., at least about any one of 85%, 90%, 93%, 95%, 97%, 98%, or 99%, or 100%) to the amino acid sequence of SEQ ID NO: 54. In some embodiments, the FAH comprises an amino acid sequence having at least about 80% (e.g., at least about any one of 85%, 90%, 93%, 95%, 97%, 98%, or 99%, or 100%) to the amino acid sequence of SEQ ID NO: 21.

In some embodiments, there is provided a method of treating Duchenne and Becker muscular dystrophy, X-linked dilated cardiomyopathy, or familial dilated cardiomyopathy in an individual, comprising administering to the individual an effective amount of a circRNA comprising a nucleic acid sequence encoding DMD (including a functional variant thereof, e.g., miniDMD), wherein the circRNA is a naked circRNA. In some embodiments, the DMD comprises an amino acid sequence having at least about 80% (e.g., at least about any one of 85%, 90%, 93%, 95%, 97%, 98%, or 99%, or 100%) to the amino acid sequence of SEQ ID NO: 23. In some embodiments, the DMD comprises an amino acid sequence having at least about 80% (e.g., at least about any one of 85%, 90%, 93%, 95%, 97%, 98%, or 99%, or 100%) to the amino acid sequence of SEQ ID NO: 22.

In some embodiments, there is provided a method of treating Ehlers-Danlos syndrome in an individual, comprising administering to the individual an effective amount of a circRNA comprising a nucleic acid sequence encoding COL3A1 (including a functional variant thereof), wherein the circRNA is a naked circRNA. In some embodiments, the COL3A1 comprises an amino acid sequence having at least about 80% (e.g., at least about any one of 85%, 90%, 93%, 95%, 97%, 98%, or 99%, or 100%) to the amino acid sequence of SEQ ID NO: 56.

In some embodiments, there is provided a method of treating Joubert syndrome in an individual, comprising administering to the individual an effective amount of a circRNA comprising a nucleic acid sequence encoding AHI1 (including a functional variant thereof), wherein the circRNA is a naked circRNA. In some embodiments, the AHI1 comprises an amino acid sequence having at least about 80% (e.g., at least about any one of 85%, 90%, 93%, 95%, 97%, 98%, or 99%, or 100%) to the amino acid sequence of SEQ ID NO: 58.

In some embodiments, there is provided a method of treating pulmonary arterial hypertension, or pulmonary veno-occlusive disease in an individual, comprising administering to the individual an effective amount of a circRNA comprising a nucleic acid sequence encoding FANCC (including a functional variant thereof), wherein the circRNA is a naked circRNA. In some embodiments, the FANCC comprises an amino acid sequence having at least about 80% (e.g., at least about any one of 85%, 90%, 93%, 95%, 97%, 98%, or 99%, or 100%) to the amino acid sequence of SEQ ID NO: 59.

In some embodiments, there is provided a method of treating primary familial hypertrophic cardiomyopathy in an individual, comprising administering to the individual an effective amount of a circRNA comprising a nucleic acid sequence encoding MYBPC3 (including a functional variant thereof), wherein the circRNA is a naked circRNA. In some embodiments, the MYBPC3 comprises an amino acid sequence having at least about 80% (e.g., at least about any one of 85%, 90%, 93%, 95%, 97%, 98%, or 99%, or 100%) to the amino acid sequence of SEQ ID NO: 60.

In some embodiments, there is provided a method of treating X-linked severe combined immunodeficiency in an individual, comprising administering to the individual an effective amount of a circRNA comprising a nucleic acid sequence encoding IL2RG (including a functional variant thereof), wherein the circRNA is a naked circRNA. In some embodiments, the IL2RG comprises an amino acid sequence having at least about 80% (e.g., at least about any one of 85%, 90%, 93%, 95%, 97%, 98%, or 99%, or 100%) to the amino acid sequence of SEQ ID NO: 61.

In some embodiments, the circRNA has a functional half-life of at least or at least about 20 hours, 24 hours, 30 hours, or 36 hours. In some embodiments, the circRNA has a duration of therapeutic effect in a human cell of at least or at least about 20 hours, 24 hours, 30 hours, or 36 hours. In some embodiments, the circRNA has a duration of therapeutic effect in a human cell greater than or equal to that of an equivalent linear RNA comprising the same expression sequence. In some embodiments, the circRNA has a functional half-life in a human cell greater than or equal to that of an equivalent linear RNA comprising the same expression sequence.

In some embodiments, the therapeutic polypeptide comprises IDUA, and the disease or condition is Hurler Syndrome. In some embodiments, administration of the circRNA restores at least 10%, at least 15%, at least 20%, at least 25%, at least 30%, at least 40%, at least 50%, at least 60%, at least 70%, at least 80%, at least 90%, or at least 100% of α-l-iduronidase in a human or an animal model with a mutation in IDUA compared to wild-type. In some embodiments, the catalytic activity of IDUA increases from 4 to 24 hours (e.g., from 4 to 8 hours, from 8 to 12 hours, from 12 to 16 hours, from 16 to 20 hours, and/or from 16 to 24 hours) following administration of the circRNA encoding IDUA. In some embodiments, the circRNA encoding IDUA has a functional half-life of at least or at least about 20 hours, 24 hours, 30 hours, or 36 hours.

### B. Treating or Preventing a Coronavirus Infection

The present application provides methods of treating or preventing a coronavirus (e.g., SARS-COV-2 infection) infection in an individual, comprising administering to the individual an effective amount of the circRNAs of any one of the embodiments described herein, wherein the circRNA encodes an antigenic polypeptide or a receptor protein (e.g., soluble receptor) of the coronavirus, or a neutralizing antibody specifically binding the coronavirus, wherein the circRNA is a naked circRNA. In some embodiments, the coronavirus is SARS-COV, MERS-COV, or SARS-COV-2. In some embodiments, the coronavirus is SARS-COV-2. In some embodiments, the present application provides methods of preventing or decreasing the risk of a coronavirus (e.g., SARS-COV-2 infection) infection in an individual, comprising administering to the individual an effective amount of the circRNA of any one of the embodiments described above, wherein the circRNA encodes an antigenic polypeptide or a receptor protein (e.g., soluble receptor) of the coronavirus, or a neutralizing antibody specifically binding the coronavirus, wherein the circRNA is a naked circRNA. In some embodiments, the method comprises administering a cocktail composition comprising a plurality of circRNAs encoding different antigenic polypeptides, receptor proteins, or neutralizing antibodies. In some embodiments, the circRNA is subject to rolling circle translation by a ribosome in the individual. In some embodiments, the circRNA is administered as naked circRNA, or as a pharmaceutical composition comprising a transfection agent.

In some embodiments, there is provided a method of treating or preventing a coronavirus (e.g., SARS-COV-2 infection) infection in an individual, comprising administering to the individual an effective amount of a circRNA comprising a nucleic acid sequence encoding a receptor protein of the coronavirus. In some embodiments, the coronavirus is SARS-COV-2, wherein the circRNA is a naked circRNA. In some embodiments, the receptor protein is a soluble receptor, such as a soluble ACE2 receptor. In some embodiments, the method comprises administering an effective amount of a cocktail composition comprising a plurality of circRNA encoding different receptor proteins.

In some embodiments, there is provided a method of treating or preventing a coronavirus (e.g., SARS-COV-2 infection) infection in an individual, comprising administering to the individual an effective amount of a circRNA comprising a nucleic acid sequence encoding a neutralizing antibody that specifically binds the coronavirus, wherein the circRNA is a naked circRNA. In some embodiments, the coronavirus is SARS-COV-2. In some embodiments, the method comprises administering an effective amount of a cocktail composition comprising a plurality of circRNA encoding different neutralizing antibodies.

In some embodiments, the present application provides methods of treating or preventing a coronavirus infection in an individual, comprising administering to the individual an effective amount of the circRNA vaccine of any one of the embodiments described herein, wherein the circRNA in the circRNA vaccine is a naked circRNA. In some embodiments, the coronavirus is SARS-COV, MERS-COV, or SARS-COV-2. In some embodiments, the coronavirus is SARS-COV-2. In some embodiments, the present application provides methods of preventing or decreasing the risk of a coronavirus (e.g., SARS-COV-2 infection) infection in an individual, comprising administering to the individual an effective amount of the circRNA vaccine of any one of the embodiments described above. In some embodiments, the circRNA is subject to rolling circle translation by a ribosome in the individual. In some embodiments, the circRNA vaccine is administered as naked circRNA, or as a pharmaceutical composition comprising a transfection agent.

In some embodiments, the present application provides methods of treating or preventing a coronavirus infection in an individual, comprising administering to the individual an effective amount of the circRNA vaccine of any one of the embodiments described herein, wherein the circRNA in the circRNA vaccine is a naked circRNA. In some embodiments, the coronavirus is a wild-type strain of SARS-COV-2 or a variant strain of SARS-COV-2. In some embodiments, the coronavirus is SARS-COV-2. In some embodiments, the SARS-COV-2 is an Alpha (B.1.1.7), Beta (B.1.351, B.1.351.2, B.1.351.3), Delta (B.1.617.2, AY.1, AY.2, AY.3), or Gamma (P.1, P.1.1, P.1.2) variant of SARS-COV-2. In some embodiments, the variant can be any variant described on cdc.gov/coronavirus/2019-ncov/variants/. In some embodiments, the present application provides methods of preventing or decreasing the risk of a coronavirus (e.g., SARS-COV-2 infection, such as an infection with any of the variant SARS-COV-2 strains described herein) infection in an individual, comprising administering to the individual an effective amount of the circRNA vaccine of any one of the embodiments described above, wherein the circRNA is a naked circRNA. In some embodiments, the circRNA vaccine encodes an S protein or fragment thereof comprising one, two, or three of the mutations selected from the group K417N, L452R, and T478K, wherein the amino acid numbering is based on SEQ ID NO: 1. In some embodiments, the circRNA vaccine encodes an S protein or fragment thereof comprising one, two, three, four, five, or more mutations selected from the group consisting of residue 69 deletion, residue 70 deletion, residue 144 deletion, E484K, S494P, N501Y, A570D, D614G, P681H, T716I, S982A, D1118H, and K1191N, wherein the amino acid numbering is based on SEQ ID NO: 1. In some embodiments, the circRNA vaccine encodes an S protein or fragment thereof comprising one, two, or three of the mutations selected from the group consisting of E484K, S494P, and N501Y, wherein the amino acid numbering is based on SEQ ID NO: 1. In some embodiments, the circRNA vaccine encodes an S protein or fragment thereof comprising one, two, three, four, five, or more mutations selected from the group consisting of D80A, D215G, 241del, 242del, 243del, K417N, E484K, N501Y, D614G, and A701V, wherein the amino acid numbering is based on SEQ ID NO: 1. In some embodiments, the circRNA vaccine encodes an S protein or fragment thereof comprising one, two, or three of the mutations selected from the group consisting of K417N, E484K, and N501Y, wherein the amino acid numbering is based on SEQ ID NO: 1. In some embodiments, the circRNA vaccine encodes an S protein or fragment thereof comprising one, two, three, four, five, or more mutations selected from the group consisting of L18F, T20N, P26S, D138Y, R190S, K417T, E484K, N501Y, D614G, H655Y, and T1027I, wherein the amino acid numbering is based on SEQ ID NO: 1. In some embodiments, the circRNA vaccine encodes an S protein or fragment thereof comprising one, two, or three of the mutations selected from the group consisting of K417T, E484K, and N501Y.

In some embodiments, the present application provides methods of treating or preventing a multiple strains of a coronavirus (e.g., multiple strains of SARS-COV-2) infection in an individual, comprising administering to the individual an effective amount of the circRNA vaccine of any one of the embodiments described herein, wherein the circRNA in the circRNA vaccine is a naked circRNA. In some embodiments, the present application provides methods of treating or preventing a multiple strains of a coronavirus (e.g., multiple strains of SARS-COV-2) infection in an individual, comprising administering to the individual an effective amount of multiple different circRNA vaccines of any one of the embodiments described herein. In some embodiments, the method comprises administering to the individual a composition comprising a plurality (e.g., two or more) circRNAs, wherein a first circRNA encodes an S protein or fragment thereof of a first strain of a coronavirus, and a second circRNA encodes an S protein or fragment thereof of a second strain of a coronavirus, wherein the first circRNA and the second circRNA are naked circRNA. In some embodiments, at least one of the circRNAs of the plurality encodes an S protein or fragment thereof comprising the mutations found in the D614G, B.1.1.7/501Y.V1 variant of SARS-COV-2 or the B.1.351/501Y.V2 variant of SARS-COV-2.

In some embodiments, the present application provides methods of treating or preventing a coronavirus infection in an individual, comprising administering to the individual an effective amount of a circRNA vaccine comprising a circRNA comprising a nucleic acid sequence encoding an antigenic polypeptide comprising an S protein or a fragment thereof of the coronavirus (e.g., SARS-COV-2), wherein the circRNA in the circRNA vaccine is a naked circRNA. In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence selected from the group consisting of SEQ ID NOs: 11-15. In some embodiments, the circRNA is subject to rolling circle translation by a ribosome in the individual.

In some embodiments, the present application provides methods of treating or preventing a coronavirus infection in an individual, comprising administering to the individual an effective amount of a circRNA vaccine comprising a circRNA comprising: (a) a nucleic acid sequence encoding an antigenic polypeptide comprising an S protein or a fragment thereof of a coronavirus (e.g., SARS-COV-2), and (b) an IRES sequence, wherein the IRES sequence is operably linked to the nucleic acid sequence encoding the antigenic polypeptide, wherein the circRNA is naked circRNA. In some embodiments, the circRNA further comprises an in-frame 2A peptide coding sequence operably linked to the 3′ end of the nucleic acid sequence encoding the antigenic polypeptide. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the IRES sequence, the Kozak sequence, the SP, and the nucleic acid sequence encoding the antigenic polypeptide. In some embodiments, the circRNA further comprises a polyA or polyAC sequence disposed at the 5′ end of an IRES sequence. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence selected from the group consisting of SEQ ID NOs: 11-15. In some embodiments, the circRNA is subject to rolling circle translation by a ribosome in the individual. In some embodiments, the circRNA vaccine is administered via intramuscular (i.m) injection. In some embodiments, one or more doses of the circRNA vaccine are administered. In some embodiments, the interval between doses is about 2 weeks (e.g., 12, 13, 14, 15, or 16 days). In some embodiments, the method comprises administering a first dose of the circRNA vaccine and administering a second dose of the circRNA vaccine after 2 weeks or about 2 weeks.

In some embodiments, the present application provides methods of treating or preventing a coronavirus infection in an individual, comprising administering to the individual an effective amount of a circRNA vaccine comprising a circRNA comprising: (a) a nucleic acid sequence encoding an antigenic polypeptide comprising an S protein or a fragment thereof of a coronavirus (e.g., SARS-COV-2), and (b) an m6A modification motif sequence operably linked to the nucleic acid sequence encoding the antigenic polypeptide, wherein the circRNA is a naked circRNA. In some embodiments, the nucleic acid sequence further encodes a SP (e.g., human tPA or IgE SP) fused to the N-terminus of the S protein or fragment thereof. In some embodiments, the circRNA further comprises a Kozak sequence operably linked to the nucleic acid sequence encoding the antigenic polypeptide. In some embodiments, the circRNA comprises a nucleic acid sequence comprising from the 5′ end to the 3′ end: the m6A modification motif sequence, the Kozak sequence, the SP, and the nucleic acid sequence encoding the antigenic polypeptide. In some embodiments, the circRNA further comprises a 3′ exon sequence recognizable by a 3′ catalytic Group I intron fragment flanking the 5′ end of the nucleic acid sequence encoding the antigenic polypeptide, and a 5′ exon sequence recognizable by a 5′ catalytic Group I intron fragment flanking the 3′ end of the nucleic acid sequence encoding the antigenic polypeptide. In some embodiments, the circRNA further comprises a 5′ ligation sequence at the 5′ end of the circRNA, and a 3′ ligation sequence at the 3′ end of the circRNA, wherein the 5′ ligation sequence and the 3′ ligation sequence are ligated to each other via a ligase (e.g., T4 RNA ligase). In some embodiments, the antigenic polypeptide comprises a RBD of the S protein. In some embodiments, the antigenic polypeptide further comprises a multimerization domain (e.g., C-terminal Fd domain, or a GCN-4 based isoleucine zipper domain). In some embodiments, the antigenic polypeptide comprises an S2 region of the S protein. In some embodiments, the antigenic polypeptide comprises amino acid residues 2-1273 of a full-length S protein of SARS-COV-2, wherein the numbering is based on SEQ ID NO: 1. In some embodiments, the S2 region of the S protein comprises one or more mutations (e.g., K986P and V987P) that stabilize a pre-fusion conformation of the S protein. In some embodiments, the antigenic polypeptide comprises one or more mutations (e.g., deletion of amino acid residues 681-684) that inhibit cleavage of the S protein. In some embodiments, the antigenic polypeptide comprises an S protein or fragment thereof of SARS-CoV-2 having a D614G mutation. In some embodiments, the circRNA comprises a nucleic acid sequence selected from the group consisting of SEQ ID NOs: 11-15. In some embodiments, the circRNA is subject to rolling circle translation by a ribosome in the individual. In some embodiments, the circRNA vaccine is administered via intramuscular (i.m) injection. In some embodiments, one or more doses of the circRNA vaccine are administered. In some embodiments, the interval between doses is at least about 2 weeks (e.g., at least about any of 2, 3, 4, 5, 6, 7, or 8 weeks). In some embodiments, the method comprises administering a first dose of the circRNA vaccine and administering a second dose of the circRNA vaccine after at least any of 3, 4, 5, 6, 7, or 8 weeks.

### V. Method of Preparation

The circRNAs described herein can be prepared, for example, by chemical ligation, enzymatic ligation, or ribozyme autocatalysis of linear RNAs. In some embodiments, the circRNA is prepared by circularizing a linear RNA in vitro.

### Linear RNA and Nucleic Acid Constructs Encoding Thereof

In some embodiments, a linear RNA capable of forming the circRNA of any one of the embodiments described above can circularized by chemical circularization methods using cyanogen bromide or a similar condensing agent. In some embodiments, the linear RNA can be circularized by autocatalysis of a Group I intron comprising a 5′ catalytic Group I intron fragment and a 3′ catalytic Group I intron fragment. In some embodiments, the linear RNA can be circularized by a ligase. In some embodiments, the linear RNA can be circularized by a T4 RNA ligase. In some embodiments, the linear RNA can be circularized by a DNA ligase. Suitable ligases include, but are not limited to a T4 DNA ligase (T4 Dnl), a T4 RNA ligase 1 (T4 Rnl1) and a T4 RNA ligase 2 (T4 Rnl2).

In some embodiments, the linear RNA can be circularized by autocatalysis of a Group I intron. In some embodiments, the Group I intron comprises a 5′ catalytic Group I intron fragment and a 3′ catalytic Group I intron fragment. In some embodiments, the linear RNA comprises a 3′ catalytic Group I intron fragment (such as the sequence set forth in SEQ ID NO: 46) flanking the 5′ end of a 3′ exon sequence recognizable by the 3′ catalytic Group I intron fragment (such as the sequence set forth in SEQ ID NO: 39), and the 5′ catalytic Group I intron fragment (such as the sequence set forth in SEQ ID NO: 47) flanking the 3′ end of a 5′ exon sequence recognizable by the 5′ catalytic Group I intron fragment (such as the sequence set forth in SEQ ID NO: 40).

In some embodiments, the linear RNA comprises, from 5′ to 3′ end, a 3′ Intron-IRES-Kozak-SP-Spike-5′ Intron sequence. In some embodiments, the Spike sequence comprises one of the sequences set forth in SEQ ID NOs: 11-15 and SEQ ID NOs: 48-49.

In some embodiments, the linear RNA comprises, from 5′ to 3′ end, a 3′ Intron-IRES-Kozak-SP-RBD-5′ Intron sequence. In some embodiments, the RBD sequence comprises amino acid residues 319 to 542 of a full-length S protein of SARS-COV-2, wherein the numbering is based on SEQ ID NO: 1

In some embodiments, the linear RNA comprises, from 5′ to 3′ end, a 3′ Intron-IRES-Kozak-SP-nAb-5′ Intron sequence. In some embodiments, the nAb sequence encodes the amino acid sequence of SEQ ID NO: one of SEQ ID NOS: 26-35. In some embodiments, the nAb sequence encodes the amino acid sequence of SEQ ID NO: 26. In some embodiments, the nAb sequence encodes the amino acid sequence of SEQ ID NO: 27. In some embodiments, the nAb sequence encodes the amino acid sequence of SEQ ID NO: 30.

In some embodiments, the linear RNA comprises, from 5′ to 3′ end, a 3′ Intron-IRES-Kozak-IDUA-5′ Intron sequence. In some embodiments, the IDUA sequence encodes the amino acid sequence of SEQ ID NO: 18. In some embodiments, the IDUA sequence encodes the amino acid sequence of SEQ ID NO: 19.

In some embodiments, the linear RNA further comprises a 5′ homology sequence flanking the 5′ end of the 3′ catalytic Group I intron fragment, and a 3′ homology sequence flanking the 3′ end of the 5′ catalytic Group I intron fragment. In some embodiments, the linear RNA comprises, from 5′ to 3′ end, a 5′ homology arm-3′ catalytic Group I Intron fragment-3′ exon sequence-IRES-Kozak-SP-antigenic polypeptide (e.g., Spike protein or fragment thereof)-5′ exon sequence-5′ catalytic Group I Intron fragment-3′ homology arm sequence. In some embodiments, the homology sequence can be between 1 and 100, between 5 and 80, between 5 and 60, between 10 and 50, or between 12 and 50 nucleotides in length. In some embodiments, the homology sequence is about 20-30 nucleotides in length. In some embodiments, the 5′ homology sequence comprises the nucleic acid sequence of SEQ ID NO: 41, and the 3′ homology sequence comprises the nucleic acid sequence of SEQ ID NO: 42. In some embodiments, the homology arms increase the efficiency of RNA circularization by about 0 to 20%, more than 20%, more than 30%, more than 40%, or more than 50%.

In some embodiments, there is provided a nucleic acid construct comprising a nucleic acid sequence encoding the linear RNA. In some embodiments, a T7 promoter is operably linked to the nucleic acid sequence encoding the linear RNA. In some embodiments, the T7 promoter comprises the sequence set forth in SEQ ID NO: 43. In some embodiments, the T7 promoter is capable of driving in vitro transcription.

### Linear RNA Circularized by Chemical Ligation

In some embodiments, the circRNA described herein can be prepared by a method comprising: (a) chemically ligating the 5′ end and the 3′ end of a linear RNA comprising a nucleic acid sequence encoding the circRNA; and (b) isolating the circularized RNA product, thereby providing the circRNA.

In some embodiments, the step of circularizing the linear RNA comprises chemical circularization methods using cyanogen bromide or a similar condensing agent.

In some embodiments, the linear RNA can be circularized by chemical methods. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS ester reactive group and the 3′-end may contain a 3′-amino terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60, at least about 70%, at least about 80%, at least about 90%, at least about 95%, or 100%. In some embodiments, the circularization efficiency of the circularization methods provided herein is at least about 40%.

### Linear RNA Circularized by Ribozyme Autocatalysis

In some embodiments, the circRNA can be obtained by circularizing a linear RNA by ribozyme autocatalysis. In some embodiments, the linear RNA is circularized in vitro. In some embodiments, circularization by ribozyme autocatalysis comprises (a) subjecting the linear RNA to a condition that activates autocatalysis of the Group I intron (or 5′ and 3′ catalytic Group I intron fragments thereof) to provide a circularized RNA product; and (b) isolating the circularized RNA product, thereby providing the circRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the circRNA is made by a method comprising cyclizing a linear RNA (e.g., purified linear RNA) by ribozyme autocatalysis of the Group I intron. During splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron. In some embodiments, the condition that activates autocatalysis of the Group I intron or 5′ and 3′ catalytic Group I intron fragments is the addition of GTPs and Mg²⁺. In some embodiments, there is provided a step of cyclizing the linear RNAs by adding GTPs and Mg²⁺ at 55° C. for 15 min. In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, the circularization has an efficiency of at least 2%, at least 5%, at least 10%, at least 15%, at least 20%, at least 25%, at least 30%, at least 32%, at least 34%, at least 36%, at least 38%, at least 40%, at least 42%, at least 44%, at least 46%, at least 48%, or at least 50%. In some embodiments, the circularization has an efficiency of about 40% to about 50% or more than 50%.

### Linear RNA Circularized by Ligation

In some embodiments, the circRNA can be obtained by circularizing a linear RNA using a ligase such as a RNA ligase. In some embodiments, the linear RNA is circularized in vitro. In some embodiments, the linear RNA can be circularized by a T4 RNA ligase. In some embodiments, the linear RNA comprises a 5′ ligation sequence at the 5′ end of the nucleic acid sequence encoding the circRNA, and a 3′ ligation sequence at the 3′ end of the nucleic acid sequence encoding the circRNA, wherein the 5′ ligation sequence and the 3′ ligation sequence can be ligated to each other via the RNA ligase. In non-limiting examples, the linear RNA can be circularized by a ligase such as a T4 DNA ligase (T4 Dnl), T4 RNA ligase 1 (T4 Rnl1), and T4 RNA ligase 2 (T4 Rnl2). The linear RNA may be circularized with or without the presence of a single stranded nucleic acid adaptor, e.g., a splint DNA.

In some embodiments, the circRNA can be prepared by a method comprising: (a) contacting any one of the linear RNAs comprising a 5′ ligation sequence at the 5′ end of the nucleic acid sequence encoding the circRNA, and a 3′ ligation sequence at the 3′ end of the nucleic acid sequence encoding the circRNA described above with a single-stranded adaptor nucleic acid comprising from the 5′ end to the 3′ end: a first sequence complementary to the 3′ ligation sequence and a second sequence complementary to the 5′ ligation sequence, and wherein the 5′ ligation sequence and the 3′ ligation sequence hybridize to the single-stranded adaptor nucleic acid to provide a duplex nucleic acid intermediate comprising a single strand break between the 3′ end of the 5′ ligation sequence and the 5′ end of the 3′ ligation sequence; (b) contacting the intermediate with an RNA ligase under a condition that allows ligation of the 5′ ligation sequence to the 3′ ligation sequence to provide a circularized RNA product; and (c) isolating the circularized RNA product, thereby providing the circRNA.

In some embodiments, the method described herein comprises circularizing a linear RNA in vitro, comprising: (a) contacting any one of the linear RNAs comprising a 5′ ligation sequence at the 5′ end of the nucleic acid sequence encoding the circular RNA, and a 3′ ligation sequence at the 3′ end of the nucleic acid sequence encoding the circular RNA described above with an RNA ligase under a condition that allows ligation of the 5′ ligation sequence to the 3′ ligation sequence to provide a circularized RNA product; and (b) isolating the circularized RNA product, thereby providing the circular RNA.

In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear circular RNA is incubated at 37° C. for 1 hour with 1-10 units of T4 RNA ligase (New England Biolabs, Ipswich, Mass) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SPLINTR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase may be used in the synthesis of the circular RNA. As a non-limiting example, the ligase may be a circ ligase or circular ligase.

Purification of circRNA

In some embodiments, the circRNA made by the methods described herein can be further purified. In non-limiting examples, the circRNA is purified by gel-purification or by high-performance liquid chromatography (HPLC). In some embodiments, agarose gel electrophoresis allows for simple and effective separation of circular splicing products from linear precursor molecules, nicked circles, splicing intermediates, and excised introns. In some embodiments, the method comprises purifying the circular RNA by chromatography, such as HPLC. In some embodiments, the purified circular RNA can be stored at −80° C.

### VI. Pharmaceutical Compositions, Kits and Articles of Manufacture

Further provided by the present application are pharmaceutical compositions comprising any one of circRNAs described herein, and a pharmaceutically acceptable carrier. Pharmaceutical compositions can be prepared by mixing the therapeutic agents described herein having the desired degree of purity with optional pharmaceutically acceptable carriers, excipients or stabilizers (Remington's Pharmaceutical Sciences 16th edition, Osol, A. Ed. (1980), incorporated herein by reference in its entirety), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising any one of the circRNAs described herein and instructions for treating or preventing a disease or condition (e.g., coronavirus infection).

In some embodiments, there is provided a kit comprising any one of the circRNA described herein and instructions for treating or preventing a coronavirus infection.

In some embodiments, there is provided a kit comprising any one of the plasmids or linear RNAs described herein, and instructions for preparing any one of the circRNAs. In some embodiments, there is provided a kit comprising any one of the plasmids, linear RNAs, or circRNAs described herein, and instructions for administering the circRNA.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the circRNA as disclosed herein to provide effective treatment of an individual or of many individuals. Additionally, kits may be provided that contain sufficient dosages of the circRNA to allow for multiple administrations to an individual (e.g., initial vaccine administration and subsequent booster administration, in the case of a circRNA vaccine). Kits may also include multiple unit doses of the pharmaceutical compositions and instructions for use and packaged in quantities sufficient for storage and use in pharmacies, for example, hospital pharmacies and compounding pharmacies.

In some embodiments, the kit comprises a delivery system. The delivery system may be a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be syringes, dropper bottles, plastic squeeze units, atomizers, nebulizers or pharmaceutical aerosols in either unit dose or multiple dose packages. In some embodiments, there is provided a delivery system of any one of the circRNAs described herein, comprising the circRNA and a device for delivering the circRNA.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended embodiments.

### Example 1. In Vitro circRNA Production by Ligation

This example demonstrates in vitro production of a circular RNA (circRNA) by ligation.

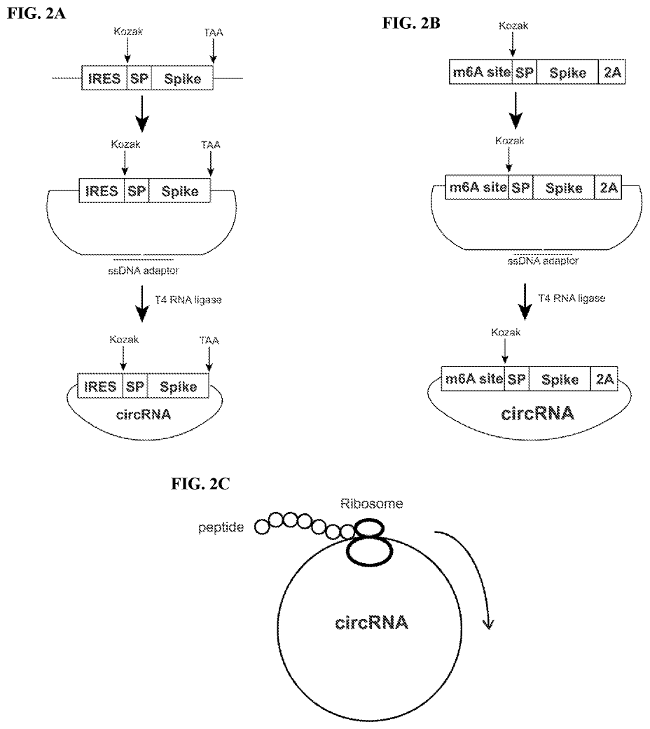

A linear RNA is designed that can be circularized to produce a circRNA comprising, from 5′ to 3′, an IRES-Kozak-SP-Spike sequence, as shown in FIG. 2A. The linear RNA is designed with, from 5′ to 3′, an IRES sequence (SEQ ID NO: 53), a Kozak sequence (SEQ ID NO: 36), a signal peptide coding sequence (SEQ ID NO: 16 or SEQ ID NO: 17), and a Spike protein coding sequence having K986P/V987P and A681-684 modifications (SEQ ID NO: 15) followed by a TAA stop codon.

Linear RNAs that can be circularized to produce the circular RNA (circRNAs) disclosed herein may be made using standard laboratory methods and materials. The cDNA sequence encoding the linear RNA may be synthesized by de novo DNA synthesis. The synthetic nucleic acid can be ordered from a synthetic nucleotide service such as GBLOCKS® (Integrated DNA Technologies). The nucleic acid sequence encoding the linear RNA sequence can be cloned into a plasmid vector containing a T7 promoter, the multiple cloning site flanked by restriction sites such as Xbal restriction sites. The resulting plasmid may be transformed into chemically competent E. coli.

For the present example, NEB DH5-alpha Competent E. coli cells are used. Transformations are performed according to NEB instructions using 100 ng of plasmid. The protocol is as follows:
1. Thaw a tube of NEB 5-alpha Competent E. coli cells on ice for 10 minutes.
2. Add 1-5 μL containing 1 pg-100 ng of plasmid DNA to the cell mixture. Carefully flick the tube
4-5 times to mix cells and DNA. Do not vortex.
3. Place the mixture on ice for 30 minutes. Do not mix.
4. Heat shock at 42° C. for exactly 30 seconds. Do not mix.
5. Place on ice for 5 minutes. Do not mix.
6. Pipette 950 μL of room temperature SOC into the mixture.
7. Place at 37° C. for 60 minutes. Shake vigorously (250 rpm) or rotate.
8. Warm selection plates to 37° C.
9. Mix the cells thoroughly by flicking the tube and inverting.

Spread 50-100 μL of each dilution onto a selection plate and incubate overnight at 37° C. Alternatively, incubate at 30° C. for 24-36 hours or 25° C. for 48 hours.

A single colony is then used to inoculate 5 ml of LB growth media using the appropriate antibiotic and then allowed to grow (250 RPM, 37° C.) for 5 hours. This is then used to inoculate a 200 ml culture medium and allowed to grow overnight under the same conditions. To isolate the plasmid (up to 850 mg), a maxi prep is performed using the Invitrogen PURELINK™ HiPure Maxiprep Kit (Carlsbad, CA), following the manufacturer's instructions.

In order to generate a linearized plasmid DNA template for In Vitro Transcription (IVT), the plasmid (an Example of which is shown in FIGS. 2A-2C) is first linearized using a restriction enzyme such as Xbal. A typical restriction digest with Xbal comprises the following: Plasmid 1.0 mg 10× Buffer 1.0 mL; Xbal 1.5 mL; dH20 up to 10 mL; incubated at 37° C. for 1 hr. If performing at lab scale (<5), the reaction is cleaned up using Invitrogen's PURELINK™ PCR Micro Kit (Carlsbad, CA) per manufacturer's instructions. Larger scale purifications may need to be done with a product that has a larger load capacity such as Invitrogen's standard PURELINK™ PCR Kit (Carlsbad, CA). Following the cleanup, the linearized vector is quantified using the NanoDrop and analyzed to confirm linearization using agarose gel electrophoresis.

Unmodified linear RNA is synthesized by in vitro transcription using T7 RNA polymerase from the linearized plasmid. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

### Example 2. In Vitro circRNA Production by Group I Ribozyme Autocatalysis

This example demonstrates in vitro production of a circular RNA (circRNA) by Group I ribozyme autocatalysis.

A linear RNA is designed that can be circularized to produce a circRNA comprising, from 5′ to 3′, a 5′ Homology arm-3′ catalytic Group I intron fragment-3′ exon sequence recognizable by the 3′ catalytic Group I intron fragment (i.e., Exon 2)-m6A modification motif-Kozak-SP-Spike-2A peptide-5′ exon sequence recognizable by the 5′ catalytic Group I intron fragment (i.e., Exon 1)-5′ catalytic Group I intron fragment-3′ Homology arm, as shown in FIG. 1C. The linear RNA is designed with, from 5′ to 3′, a 5′ homology arm (SEQ ID NO: 41), a 3′ catalytic Group I intron sequence (SEQ ID NO: 46), a 3′ exon sequence recognizable by a 3′ catalytic Group I intron fragment (SEQ ID NO: 39), a m6A modification motif sequence (SEQ ID NO: 38), a Kozak sequence (SEQ ID NO: 37), a signal peptide coding sequence (SEQ ID NO: 16 or SEQ ID NO: 17), a Spike protein coding sequence having K986P/V987P and A681-684 modifications (SEQ ID NO: 15), a 2A peptide coding sequence (SEQ ID NO: 44 or SEQ ID NO: 45), a 5′ exon sequence recognizable by a 5′ catalytic Group I intron fragment (SEQ ID NO: 40), a 5′ catalytic Group I intron fragment (SEQ ID NO: 47), and a 3′ homology arm (SEQ ID NO: 43).

Linear RNAs that can be circularized to produce the circular RNA (circRNAs) disclosed herein may be made by the same methods described in Example 1 above.

Circularized RNA is generated by ribozyme autocatalysis of the Group I intron. During splicing, the 3′ hydroxyl group of a guanosine nucleotide engages in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron.

Unmodified linear mRNA or circRNA precursors are synthesized by in-vitro transcription from a linearized plasmid DNA template using a T7 High Yield RNA Synthesis Kit (New England Biolabs). After in vitro transcription, reactions are treated with DNase I (New England Biolabs) for 20 min. After DNase treatment, unmodified linear mRNA is column purified using a MEGAclear Transcription Clean-up kit (Ambion). RNA is then heated to 70° C. for 5 min and immediately placed on ice for 3 min, after which the RNA is capped using mRNA cap-2′-O-methyltransferase (NEB) and Vaccinia capping enzyme (NEB) according to the manufacturer's instructions. Polyadenosine tails are added to capped linear transcripts using E. coli PolyA Polymerase (NEB) according to manufacturer's instructions, and fully processed mRNA is column purified. For circRNA, after DNase treatment additional GTP is added to a final concentration of 2 mM, and then reactions are heated at 55° C. for 15 min. RNA is then column purified. In some cases, purified RNA is recircularized: RNA is heated to 70° C. for 5 min and then immediately placed on ice for 3 min, after which GTP is added to a final concentration of 2 mM along with a buffer including magnesium (50 mM Tris-HCl, 10 mM MgCl2, 1 mM DTT, pH 7.5; New England Biolabs). RNA is then heated to 55° C. for 8 min, and then column purified. To enrich for circRNA, 20 μg of RNA is diluted in water (86 μL final volume) and then heated at 65° C. for 3 min and cooled on ice for 3 min. 20U RNase R and 10 μL of 10× RNase R buffer (Epicenter) is added, and the reaction is incubated at 37° C. for 15 min; an additional 10U RNase R is added halfway through the reaction. RNase R-digested RNA is column purified. RNA is separated on precast 2% E-gel EX agarose gels (Invitrogen) on the E-gel iBase (Invitrogen) using the E-gel EX 1-2% program; ssRNA Ladder (NEB) is used as a standard.

For gel extractions, bands corresponding to the circRNA are excised from the gel and then extracted using a Zymoclean Gel RNA Extraction Kit (Zymogen). For high-performance liquid chromatography, 30 μg of RNA is heated at 65° C. for 3 min and then placed on ice for 3 min. RNA was run through a 4.6×300 mm size-exclusion column with particle size of 5 μm and pore size of 200 Å (Sepax Technologies; part number: 215980P-4630) on an Agilent 1100 Series HPLC (Agilent). RNA is run in RNase-free TE ss (10 mM Tris, 1 mM EDTA, pH: 6) at a flow rate of 0.3 mL/minute. RNA is detected by UV absorbance at 260 nm. Resulting RNA fractions are precipitated with 5 M ammonium acetate, resuspended in water, and then in some cases treated with RNase R as described above.

The resulting circRNA is shown in FIG. 1C.

### Example 3. Gel-Electrophoresis and RNase R Resistance of circRNA

This example demonstrates that the purity and endonuclease resistance of the purified circRNA.

First, circRNA a circRNA construct was designed comprising a nucleotide sequence encoding an RBD of a SARS-COV-2 Spike protein, using the circRNA backbone as described in Examples 1 and 2 above.

Briefly, linear RNAs were designed that can be circularized to produce a circRNA, the linear RNAs comprising, from 5′ to 3′, a 5′ Homology arm-3′ catalytic Group I intron fragment-3′ exon sequence recognizable by the 3′ catalytic Group I intron fragment (i.e., Exon 2)-IRES-Kozak-SP-RBD-TAA stop codon-5′ exon sequence recognizable by the 5′ catalytic Group I intron fragment (i.e., Exon 1)-5′ catalytic Group I intron fragment-3′ Homology arm. The linear RNA is designed with, from 5′ to 3′, a 5′ homology arm (SEQ ID NO: 41), a 3′ catalytic Group I intron sequence (SEQ ID NO: 46), a 3′ exon sequence recognizable by a 3′ catalytic Group I intron fragment (SEQ ID NO: 39), an IRES sequence (SEQ ID NO: 53), a Kozak sequence (SEQ ID NO: 37), a signal peptide coding sequence (SEQ ID NO: 16 or SEQ ID NO: 17), a Spike protein RBD sequence encoding the amino acid sequence shown in SEQ ID NO: 2 or a Spike protein sequence encoding the amino acid sequence set forth in SEQ ID NO: 63, a stop codon, a 5′ exon sequence recognizable by a 5′ catalytic Group I intron fragment (SEQ ID NO: 40), a 5′ catalytic Group I intron fragment (SEQ ID NO: 47), and a 3′ homology arm (SEQ ID NO: 43). The circularized RNA produced from this linear RNA were termed circRNA^{RBD} and circRNA^{Spike}, respectively. As a control, the 3′ Intron sequence was mutated to a random sequence to prevent circularization of the RNA, and the resulting construct was termed LinRNA^{RBD}.

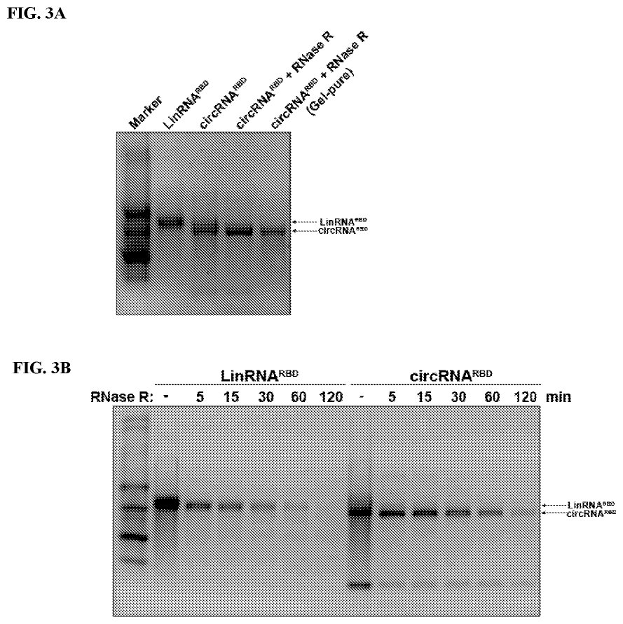

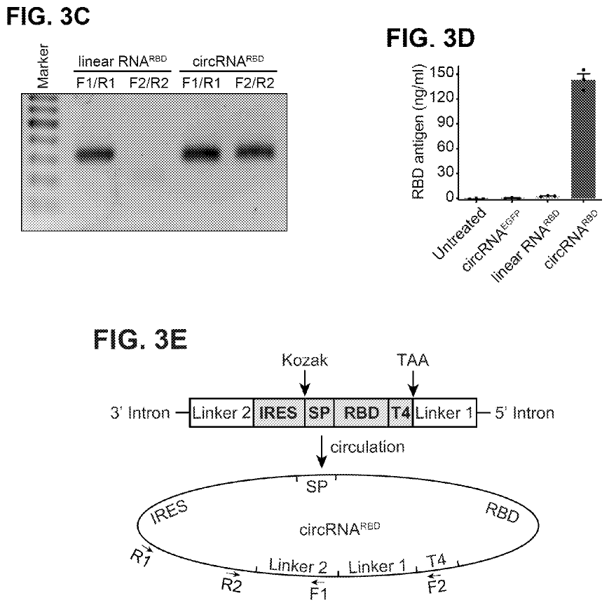

A circRNA was generated and purified as described in Example 2. The purified circRNA^{RBD} and precursor linear RNA (LinRNA^{RBD}, wherein the 3′ Intron sequence was mutated to random sequence) were resolved in agarose gel electrophoresis. The gel electrophoresis results showed that the circRNA^{RBD} ran faster than LinRNA-RBD (FIG. 3A), indicating that the RNA was circularized. The circRNA^{RBD} is a circRNA that encodes the RBD domain of the Spike protein of SARS-COV-2. The RBD domain is the amino acid residues 319 to 542 of a full-length Spike protein of SARS-COV-2, as shown in SEQ ID NO: 2. The circularization of circRNA^{RBD} was verified by reverse transcription and RT-PCR analysis (FIG. 3C) using specific primers shown in FIG. 3E.

Next, the endonuclease resistance of the purified circRNA construct was tested. Because the circRNA has no 5′ or 3′ end, the circRNA is resistant to endonuclease. The endonuclease RNase R was used to digest the circRNA^{RBD} or LinRNA^{RBD} for different times, and the reaction products were resolved in agarose gel electrophoresis. The gel electrophoresis results showed than the circRNA^{RBD} was more resistant to RNase R compared to the LinRNA^{RBD} (FIG. 3B).

### Example 4. Expression of SARS-COV-2 RBD Antigen Via circRNA Transfection in Human

HEK293T Cells and Mouse NIH3T3 Cells

This example demonstrates the ability of the circRNA to express a protein (e.g., a SARS-COV-2 RBD of a S protein) in eukaryotic cells. Additionally, this example demonstrates the surprising stability of the circRNA for two weeks at room temperature. After two weeks incubation of the circRNA at room temperature, the encoded protein could still be expressed and secreted in cells transfected with the circRNA.

After purification of the circRNA (RNase R treatment and HPLC), the circRNA^{RBD} was transfected into human HEK293T cells and mouse NIH3T3 cells with the Lipofectamine MessengerMAX Transfection Reagent (Thermo LMRNA003). The circRNA-EGFP and precursor linear RNA named LinRNA-RBD were used as controls. Quantitative ELISA assay showed that the RBD protein reached ˜143 ng/mL in the supernatant, 50-fold more than the linear RNA^{RBD} group (FIG. 3D).

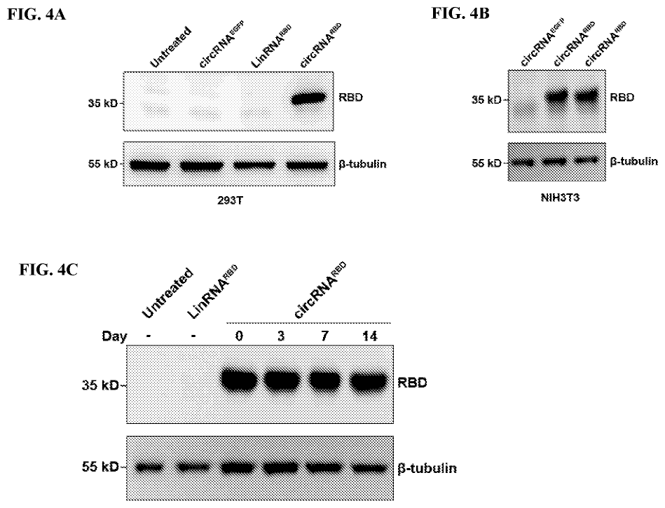

After 48 hours, the culture supernatant of transfected cells was collected for Western Blot analysis. Using the SARS-COV-2 Spike RBD antibody (ABclonal, A20135) for detection, the Western Blot results showed that the circRNA^{RBD} could express and secret the SARS-COV-2 RBD antigen to the cellular supernatant efficiently. The Western Blot results are shown in FIG. 4A and FIG. 4B.

The circRNA was stable at room temperature about 25° C. The purified circRNA^{RBD}, was kept at room temperature about 25° C. for 3, 7 or 14 days, and then were transfected into human HEK293T cells. The Western Blot results showed that the circRNA^{RBD} could express and secret the SARS-COV-2 RBD antigen to the cellular supernatant efficiently, even when the circRNA^{RBD} had been kept at room temperature for 14 days. The results are shown in FIG. 4C.

The stability of the circRNA at room temperature holds advantages for applications including vaccines and gene therapy, including for storage and transportation of the therapeutic circRNA (e.g., the circRNA vaccine).

### Example 5. The SARS-COV-2 RBD Antigen was Functional and could Block the Infection of SARS-COV-2 Pseudovirus

This example demonstrates that a secreted SARS-COV-2 RBD antigen expressed from an exemplary circRNA can directly interfere with infection of ACE2-expressing cells by a SARS-COV-2 pseudovirus.

To evaluate whether the secreted SARS-COV-2 RBD antigen produced by circRNA was functional, the cellular supernatant of HEK293T cells transfected with circRNA^{RBD} or control circRNA were incubated with the lentivirus-based SARS-COV-2 pseudovirus encoding EGFP at 37° C. for 2 hours, and then the resulting SARS-COV-2 pseudovirus/supernatant mixtures were added into the culture medium of ACE2-overexpressing cells named HEK293-ACE2. After 48 hours, the cells were collected for FACS analysis, as the SARS-COV-2 pseudovirus expressed EGFP fluorescence marker. Cellular expression of EGFP indicated infection of cells by the SARS-COV-2 pseudovirus. The commercial SARS-COV-2 neutralizing antibody (ABclonal, A19215) was used as a positive control for neutralization of the SARS-COV-2 S protein.

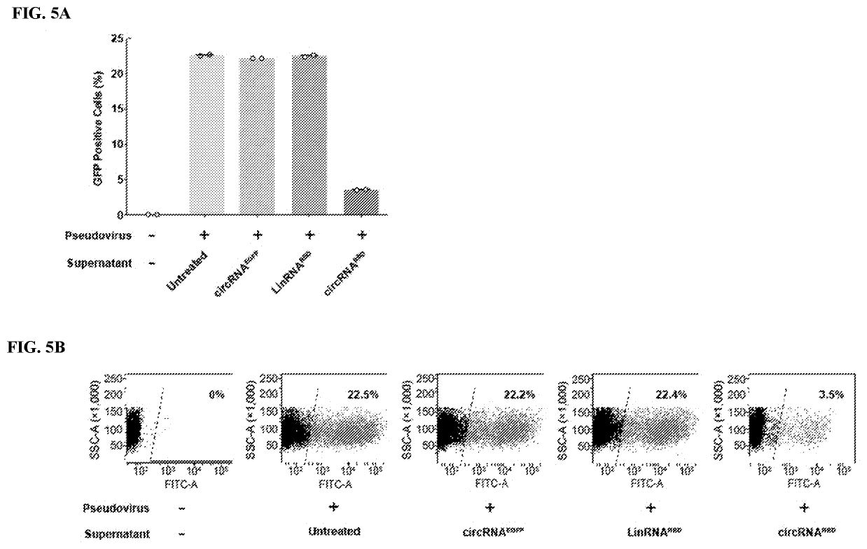

This pseudovirus competition experiment demonstrated that the secreted SARS-CoV-2 RBD antigen in supernatant produced by cells transfected with the circRNA^{RBD} could block the infection of SARS-COV-2 pseudovirus efficiently, indicating that the circRNA-produced SARS-COV-2 RBD antigen was functional at the cellular level. The secreted RBD antigen was able to interfere with binding between the RBD of the SARS-COV-2 pseudovirus, thus blocking infection of the cells. The results are shown in FIG. 5A and FIG. 5B.

### Example 6. Effects of the circRNA Vaccine

As demonstrated in Example 5 above, an RBD antigen expressed from an exemplary circRNA can directly interfere with binding of the SARS-COV-2 S protein with the ACE2 receptor, thereby preventing or reducing infection of cells by a SARS-COV-2 pseudovirus. It is expected that an antigenic polypeptide (e.g., an RBD of a coronavirus S protein) expressed from a circRNA described herein administered in vivo may stimulate a specific immune response and generate a high level of neutralizing antibodies, thus serving as effective vaccines against viruses such as coronaviruses (e.g., SARS-COV-2).

Purified circRNA^{RBD} (the circRNA backbone as shown in FIG. 1B comprising a nucleotide sequence encoding the amino acid sequence shown in SEQ ID NO: 2 as the “Spike” in FIG. 1B) and circRNA^{Spike} (the circRNA backbone as shown in FIG. 1B comprising a nucleotide sequence encoding the amino acid sequence shown in SEQ ID NO: 62 as the “Spike” in FIG. 1B) are used to immunize BALB/c mice, respectively. The first immunization is conducted via intramuscular injection at day 0, and a second dose is adopted to boost the immune response at day 14. The serum of the immunized mice is collected on day 28 for the detection of RBD specific IgG titer.

RBD specific IgG titer is measured with ELISA. The Placebo group is not expected to show RBD-specific IgG signal. In vitro surrogate neutralizing assay is further used to measure the neutralization activity of immunized mouse serum. The lentivirus-based SARS-CoV-2 pseudovirus coated with SARS-COV-2 spike protein is then used to evaluate the neutralizing activity at the cell level. The serum of immunized mice is incubated with SARS-CoV-2 pseudovirus, and then incubation system is added into the culture of ACE2-over-expression HEK293T cells. 48 hours later, the reporter-luciferase activity of pseudovirus is measured. Both the circRNA^{RBD} and circRNA^{Spike} are expected to induce SARS-COV-2 spike specific neutralizing antibody to block the infection of pseudovirus.

This Example may demonstrate that circRNA vaccine could induce SARS-COV-2 specific immune response and generate a high level of SARS-COV-2 spike specific neutralizing antibody.

### Example 7. The Effects of the circRNA Vaccine on the Weight of Mouse Spleen

This example demonstrates the effect of immunization with an exemplary circRNA encoding an antigenic polypeptide (circRNA^{RBD}) on the weight of the spleen in mice following two-dose immunization.

The circRNA dosing scheme is shown in Example 6. Four weeks after the second dose of circRNA vaccine or placebo, mice are sacrificed and the spleens of immunized mice are isolated. Then the weight of each mouse is measured. The weight of spleen from circRNA^{RBD} administered mice is expected to be higher than the placebo group.

### Example 8. Expression of SARS-COV-2 Neutralizing Antibody Via circRNA

This example demonstrates expression of a secreted virus neutralizing antibody using exemplary circRNAs. Neutralizing antibodies expressed and secreted from cells transfected with the circRNAs described herein could effectively block infection by a SARS-CoV-2 pseudovirus.

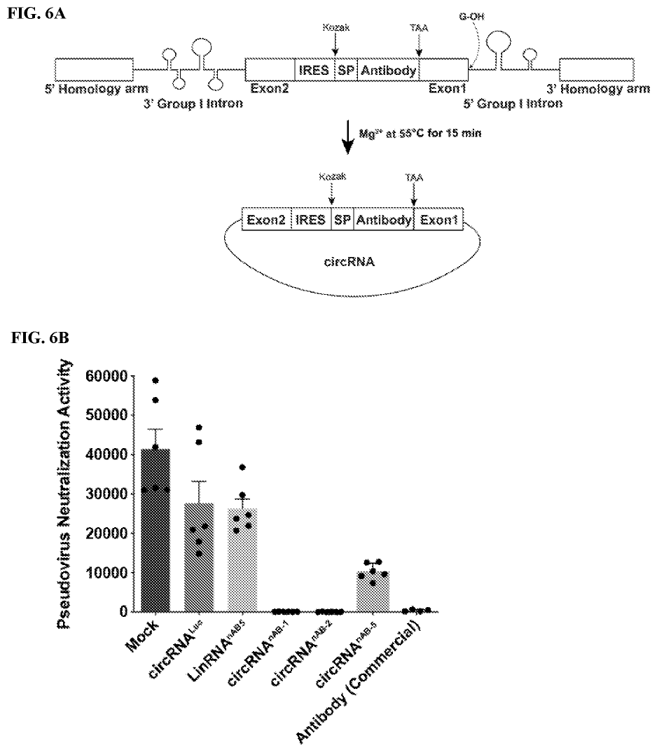

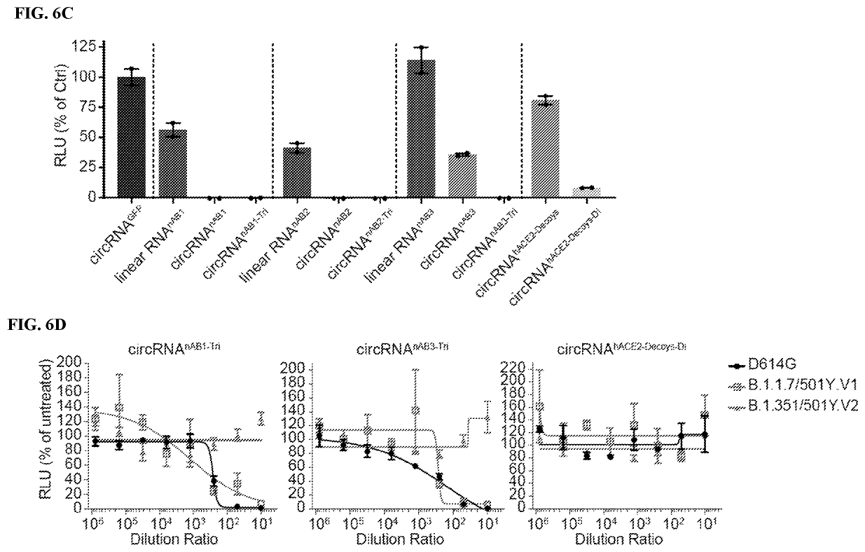

The circRNA could also be used to express SARS-COV-2 neutralizing antibody. Similar to the above RBD antigen, the SARS-COV-2 neutralizing antibody-coding sequence was also circularized via the above circularization method (FIG. 6A).

Linear RNAs were designed that can be circularized to circRNAs, the linear RNA comprising, from 5′ to 3′, a 5′ Homology arm-3′ catalytic Group I intron fragment-3′ exon sequence recognizable by the 3′ catalytic Group I intron fragment (i.e., Exon 2)-IRES-Kozak-SP-RBD-TAA stop codon-5′ exon sequence recognizable by the 5′ catalytic Group I intron fragment (i.e., Exon 1)-5′ catalytic Group I intron fragment-3′ Homology arm. The linear RNA is designed with, from 5′ to 3′, a 5′ homology arm (SEQ ID NO: 41), a 3′ catalytic Group I intron sequence (SEQ ID NO: 46), a 3′ exon sequence recognizable by a 3′ catalytic Group I intron fragment (SEQ ID NO: 39), an IRES sequence (SEQ ID NO: 53), a Kozak sequence (SEQ ID NO: 37), a signal peptide coding sequence (SEQ ID NO: 16 or SEQ ID NO: 17), a nucleotide sequence encoding a nAb (nAb-1 (the amino acid sequence shown in SEQ ID NO: 27), nAb-2 (the amino acid sequence shown in SEQ ID NO: 28), or nAb-5 (the amino acid sequence shown in SEQ ID NO: 30)), a stop codon, a 5′ exon sequence recognizable by a 5′ catalytic Group I intron fragment (SEQ ID NO: 40), a 5′ catalytic Group I intron fragment (SEQ ID NO: 47), and a 3′ homology arm (SEQ ID NO: 43). The circularized RNAs produced from these linear RNAs were termed circRNA^{nAb-1} circRNA^{nAb-2}, and circRNA^{nAB-5}, respectively. As a control, the 3′ Intron sequence of the linear construct designed to generate circRNA^{nAB-5} was mutated to a random sequence to prevent circularization of the RNA, and the resulting construct was termed LinRNA^{nAB-5}.

Circular RNAs were generated comprising a nucleotide sequence encoding nAb-1, nAb-2, nAb-3, nAb-4, nAb-5, nAb-6, nAb-7H, or nAb-7L. The amino acid sequences of the neutralizing antibodies are shown in SEQ ID NOs: 26-33, respectively. Alternatively, an antibody that binds to ACE2 and blocks binding of the S protein can be used, such as the amino acid sequences shown in SEQ ID NO: 34 or 35.

Exemplary circRNAs encoding nAbs (circRNA^{nAb-1}, circRNA^{nAb-2}, and circRNA^{nAB-5}) were transfected into HEK293T cells, and 48 hours later the supernatant was collected and used to conduct the with SARS-COV-2 pseudovirus neutralization assay. A circRNA encoding luciferase (circRNA^{Luc}) and a linear precursor RNA LinRNA^{nAB-5} were used as negative controls, and the commercial SARS-COV-2 neutralizing antibody (ABclonal, A19215) was used as the positive control.

The pseudovirus neutralization assay results demonstrated that circRNA^{nAb-1}, circRNA^{nAb-2}, and circRNA^{nAB-5} could neutralize the infection of SARS-COV-2 pseudovirus compared to negative controls (FIG. 6B). These results indicate that the circRNA can be utilized to express neutralizing antibodies for therapeutic purposes, such as to treat a coronavirus (e.g., SARS-COV-2) infection. Pseudovirus neutralization assays showed that supernatants of HEK293T cells transfected with circRNA^{nAB} or circRNA^{hACE2} decoys could effectively inhibit wild SARS-COV-2 S-protein based pseudovirus infection (FIG. 6C).

Next, neutralizing antibodies against the SARS-COV-2 variants, including B.1.1.7/501Y.V1 and B.1.351/501Y.V2, were tested by pseudovirus assays. The supernatants of circRNA^{nAB1-Tri} and circRNA^{nAB3-Tri} transfected cells effectively blocked B1.1.7/501Y.V1 and D614G pseudovirus infection (FIG. 6D). However, both nanobodies showed markedly decreased neutralizing activity against B.1.351/501Y.V2 variant (FIG. 6D). The hACE2 decoys showed no inhibition activity against B1.1.7/501Y.V1 and B.1.351/501Y.V2 variants (FIG. 6D).

In this example, circRNA-encoded SARS-COV-2 nanobodies showed strong neutralizing ability against the SARS-COV-2 native, D614G and B.1.1.7/501Y.V1 strains in vitro, but they were completely escaped by B.1.351/501Y.V2 variant (FIG. 6D). Beyond viral receptors, this circRNA expression platform hold the potential to become therapeutic drugs, encoding therapeutic antibodies in vivo, e.g. anti-PD1/PD-L1 antibodies. Compared to the antibodies protein drugs, the circRNAs could target intracellular targets, such as TP5383 and KRAS84, because they encoded therapeutic antibodies in the cytoplasm, bypassing the cytomembrane barrier.

### Example 9. A circRNA Encoding IDUA could Restore the Catalytic Activity of α-l-Iduronidase (IDUA) in Primary Cells from Hurler Syndrome Mouse Models

The above examples describe exemplary circRNA backbones, generation and purification of circRNAs, use of circRNAs to produce antigenic polypeptides for use as vaccines, and circRNA expression of neutralizing antibodies to treat an infection (e.g., a SARS-COV-2 infection). However, the circRNAs described herein can also be applied to therapy of other diseases that would benefit from expression of a therapeutic polypeptide, such as genetic diseases associated with a deficiency in a protein or functional protein. This example demonstrates that the circRNA can be used to express a functional protein such as an enzyme (e.g., IDUA). Therefore, the circRNAs provided herein can be used for production of functional therapeutic polypeptides for gene therapy applications.

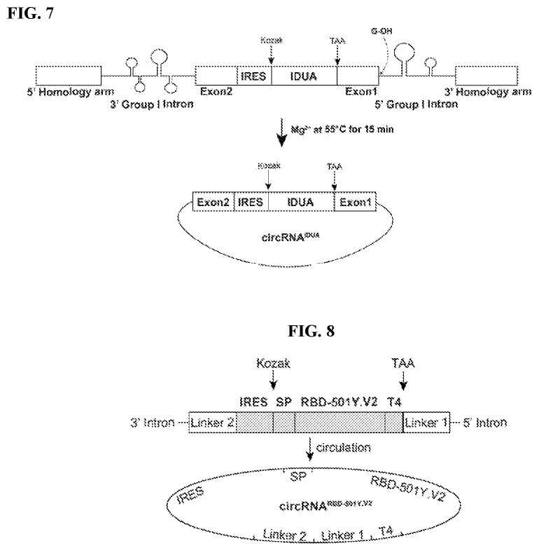

Instead of the SARS-COV-2 RBD/Spike antigen, the functional wildtype disease-related proteins can also be expressed and function via the circRNAs and methods described herein. In an example, the mouse α-l-iduronidase (IDUA) coding sequence was inserted into the circRNA backbone to generate circRNA^{IDUA} (FIG. 7).

Briefly, a linear RNA was designed that can be circularized to produce a circRNA, the linear RNAs comprising, from 5′ to 3′, a 5′ Homology arm-3′ catalytic Group I intron fragment-3′ exon sequence recognizable by the 3′ catalytic Group I intron fragment (i.e., Exon 2)-IRES-Kozak-SP-RBD-TAA stop codon-5′ exon sequence recognizable by the 5′ catalytic Group I intron fragment (i.e., Exon 1)-5′ catalytic Group I intron fragment-3′ Homology arm. The linear RNA is designed with, from 5′ to 3′, a 5′ homology arm (SEQ ID NO: 41), a 3′ catalytic Group I intron sequence (SEQ ID NO: 46), a 3′ exon sequence recognizable by a 3′ catalytic Group I intron fragment (SEQ ID NO: 39), an IRES sequence (SEQ ID NO: 53), a Kozak sequence (SEQ ID NO: 37), a signal peptide coding sequence (SEQ ID NO: 16 or SEQ ID NO: 17), a nucleotide sequence encoding IDUA (amino acid sequence set forth in SEQ ID NO: 18), a stop codon, a 5′ exon sequence recognizable by a 5′ catalytic Group I intron fragment (SEQ ID NO: 40), a 5′ catalytic Group I intron fragment (SEQ ID NO: 47), and a 3′ homology arm (SEQ ID NO: 43). The circularized RNA produced from this linear RNA was termed circRNA^{IDUA}. As a control, the 3′ Intron sequence was mutated to a random sequence to prevent circularization of the RNA, and the resulting construct was termed LinRNA^{IDUA}.

The circRNA^{IDUA} was circularized and purified according to the method described in Example 2. Purified circRNA^{IDUA} is transfected into primary MEF cells from Hurler Syndrome Mouse models or human HEK293T/IDUA^{-/-} cells.

After 48 hours, the catalytic activity of α-l-iduronidase is detected with the reported α-l-iduronidase assay (Qu et al., Nature Biotechnology, vol 37, September 2019, 1059-1069, incorporated herein by reference). The α-l-iduronidase assay is expected to show that the circRNA^{IDUA} may recover the catalytic activity of α-l-iduronidase efficiently in the primary MEF cells from Hurler Syndrome mouse models and as well as the human HEK293T/IDUA^{-/-} cells. This would indicate that the circRNA^{IDUA} is functional in Hurler Syndrome Mouse-derived primary cells.

### Example 10. In Vivo Restoration of the Catalytic Activity of α-l-Iduronidase in Hurler Syndrome Mouse Models

Example 9 above demonstrates that an exemplary circRNA may express a functional enzyme protein in mouse or human cells deficient for the enzyme, thereby restoring the protein function in those cells. The present example demonstrates that an exemplary circRNA may be used to restore function of a protein in vivo, or maybe even for an extended period (e.g., at least 24 hours).

The purified circRNA^{IDUA} (30 μg per dose) is delivered into Hurler Syndrome (IDUA deficient) mice via tail-vein injection. After 4 hours or 24 hours, the Hurler Syndrome mice are sacrificed to isolate the liver tissues, and α-l-iduronidase activity is assayed in the isolated liver tissues.

The α-l-iduronidase assay from the livers of mice injected with circRNA^{IDUA} is expected to show that circRNA^{IDUA} may efficiently restore the catalytic activity of α-l-iduronidase in Hurler Syndrome mouse models. The catalytic activity may increase from 4 hours to 24 hours, which may indicate that the effect of circRNA^{IDUA} is long lasting and may be utilized for the therapy of genetic diseases.

### Example 11. SARS-COV-2 circRNA Vaccines May Elicit Sustained Humoral Immune Responses with High-Level Neutralizing Antibodies

With its stability and immunogen-coding capability, naked circRNA could be developed into a new type of vaccine. BALB/c mice are immunized with naked circRNA^{RBD} through intramuscular injection twice, using a dose of 10 μg or 50 μg per mouse at a two-week interval, while PBS injection is used as the placebo. The amount of RBD-specific IgG and pseudovirus neutralization activity are evaluated at two or five weeks post circRNA^{RBD} boost.

High titers of RBD-specific IgG are expected to be elicited by circRNA^{RBD} vaccine in a dose-dependent manner, and for both 2- and 5-weeks post boost. These may indicate that naked circRNA^{RBD} can induce long-lasting antibodies against SARS-COV-2 RBD.

To test the antigen-specific binding capability of IgG from vaccinated animals, a surrogate neutralization assay is performed. Antibodies elicited by circRNA^{RBD} vaccines are expected to show evident neutralizing capacity in dose-dependent manner.

Sera from circRNA^{RBD}-vaccinated mice are expected to neutralize both SARS-COV-2 pseudovirus and authentic SARS-COV-2 virus. A large amount of RBD-specific IgG, potent RBD antigen neutralization, and sustained SARS-COV-2 neutralizing capacity may suggest that circRNA^{RBD} vaccines can induce a long-lasting humoral immune response in mice.

### Example 12. SARS-COV-2 circRNA Vaccines May Induce Strong T Cell Immune Responses in the Spleen

B cells (the source of antibodies), CD4⁺ T cells, and CD8⁺ T cells are three pillars of adaptive immunity, and they mediated effector functions that have been associated with the control of SARS-COV-2 in both non-hospitalized and hospitalized cases of COVID-19.

To probe CD4⁺ and CD8⁺ T cell immune responses in circRNA^{RBD} vaccinated mice (5 weeks post-boost), splenocytes are stimulated with SARS-COV-2 Spike RBD pooled peptides (Table B below), and cytokine-producing T cells are quantified by intracellular cytokine staining among effector memory T cells (Tem, CD44⁺CD62L⁻). Stimulated with RBD peptide pools, CD4⁺ T cells of mice immunized with circRNA^{RBD} vaccines are expected to exhibit Th1-biased responses, producing interferon-γ (IFN-γ), tumor necrosis factor (TNF-α), and interleukin-2 (IL-2), but not interleukin-4 (IL-4). This may indicate that circRNA^{RBD} vaccines can mainly induced Th1—but not Th2-biased immune responses. Multiple cytokine-producing CD8⁺ are also expected to be detected in circRNA^{RBD} vaccinated mice. Neutralizing antibodies in B cell responses may also been observed.

TABLE B
Peptide sequences of RBD antigens

| Name | Sequence | SEQ ID NO |
|---|---|---|
| S-45 | GIYQTSNFRVQPTESIVR | 65 |
| S-46 | RVQPTESIVRFPNITNL | 66 |
| S-47 | IVRFPNITNLCPFGEVF | 67 |
| S-48 | TNLCPFGEVFNATRFASV | 68 |
| S-49 | VFNATRFASVYAWNRKRI | 69 |
| S-50 | SVYAWNRKRISNCVADY | 70 |
| S-51 | KRISNCVADYSVLYNSA | 71 |
| S-52 | ADYSVLYNSASFSTFKCY | 72 |
| S-53 | SASFSTFKCYGVSPTKL | 73 |
| S-54 | KCYGVSPTKLNDLCFTNV | 74 |
| S-55 | KLNDLCFTNVYADSFVIR | 75 |
| S-56 | NVYADSFVIRGDEVRQIA | 76 |
| S-57 | IRGDEVRQIAPGQTGKIA | 77 |
| S-58 | IAPGQTGKIADYNYKL | 78 |
| S-59 | GKIADYNYKLPDDFTGCV | 79 |
| S-60 | KLPDDFTGCVIAWNSNNL | 80 |
| S-61 | CVIAWNSNNLDSKVGGNY | 81 |
| S-62 | NLDSKVGGNYNYLYRLFR | 82 |
| S-63 | NYNYLYRLFRKSNLKPF | 83 |
| S-64 | LFRKSNLKPFERDISTEI | 84 |
| S-65 | PFERDISTEIYQA | 85 |
| S-66 | RDISTEIYQAGSTPCNGV | 86 |
| S-67 | YQAGSTPCNGVEGFNCYF | 87 |
| S-68 | NGVEGFNCYFPLQSYGF | 88 |
| S-69 | CYFPLQSYGFQPTNGVGY | 89 |
| S-70 | GFQPTNGVGYQPYRVVVL | 90 |
| S-71 | GYQPYRVVVLSFELLHA | 91 |
| S-72 | VVLSFELLHAPATVCGPK | 92 |
| S-73 | HAPATVCGPKKSTNLVK | 93 |
| S-74 | GPKKSTNLVKNKCVNFNF | 94 |
| S-75 | VKNKCVNFNFNGLTGTGV | 95 |

These results may demonstrate that SARS-COV-2 circRNA^{RBD} vaccines can induce high level of humoral and cellular immune responses in mice. Naked circRNA^{RBD-501Y.V2} immunized mice may produce high titers of neutralizing antibodies. Given that K417N-E484K-N501Y mutant in RBD reduces its interactions with certain neutralizing antibodies as shown in Example 8, neutralizing antibodies produced by mice immunized with naked circRNA^{RBD} or naked circRNA^{RBD-501Y.V2} may show preferential neutralizing abilities to their corresponding virus strains. Recent studies suggested that 501Y.V2 showed no higher infectivity but had immune escape capability, and varieties of vaccines were reported to be less effective against SARS-COV-2 variants. There have also been reported vaccine breakthrough infections with SARS-COV-2 variants. Thus, it is important to develop and implement vaccines against emerging variants, and the circRNA vaccine may be such a platform that could be rapidly tailored for specific variants. For example, a vaccine contains E484K, N501Y, and L452R mutations in the RBD can be developed quickly via the circRNA vaccine platform to deal with a potential outbreak caused by the SARS-COV-2 variants (L452R being found in the recently reported B.1.617 276 variant emerging in India and in the B.1.429 variant that has emerged in the USA).

This generalizable strategy can be used for designing immunogens. The coding sequence of circular RNA can be quickly adapted to deal with any emerging SARS-COV-2 variants, such as the recently reported B.1.1.7/501Y.V1, B.1.351/501Y.V2, P.1/501Y.V3 and B.1.671 variants. Moreover, circular RNAs could be quickly generated in large quantities in vitro, and they do not require any nucleotide modification.

### Example 13. SARS-COV-2 circRNA^{RBD-501Y.v2} Vaccines Elicited Antibodies May Show Preferential Neutralizing Activity Against B.1.351 Variant

To evaluate the efficacy of a circRNA vaccine encoding RBD/K417N-E484K-N501Y derived from the B.1.351/501Y.V2 variant, termed as circRNA^{RBD-501Y.V2.V2}, circRNA^{RBD-501Y.V2} was constructed (FIG. 8). BALB/c mice are immunized with an i.m. injection of the circRNA^{RBD-501Y.V2} vaccine, followed by a boost at a two-week interval. The immunized mice's sera are collected at 1 and 2 weeks post the boost. RBD-501Y.V2-specific IgG titer is expected to be higher than the placebo group by ELISA. The surrogate neutralization assay is expected to show that sera of circRNA^{RBD-501Y.V2} immunized mice can effectively neutralize RBD antigens. The neutralization activity of the sera from mice immunized with circRNA^{RBD} or circRNA^{RBD}-501Y.V2 vaccines against D614G, B.1.1.7/501Y.V1, or B.1.351/501Y.V2 variants are tested. VSV-based pseudovirus neutralization assay is expected to show that antibodies elicited by circRNA^{RBD} vaccines, which encode the native RBD sequence, may effectively neutralize all three viral strains. The circRNA^{RBD-501Y.V2} immunized mouse serum is expected to neutralize its corresponding variant 501Y.V2, and possibly all three pseudoviruses.

The neutralizing capacity of the circRNA^{RBD-501Y.V2} immunized mouse serum against authentic SARS-COV-2 strains are further tested. The serum from immunized mice is expected to effectively neutralize authentic SARS-COV-2 B.1.351/501Y.V2 strain. CircRNA vaccines-elicited antibodies may show the best neutralizing activity against their corresponding variant strains. Vaccines for corresponding variant strains or multivalent vaccines might provide better protection for both native SARS-COV-2 strain and its circulating variants.

### Example 14. circRNA^{RBD-501Y.v2} Vaccines Against Authentic B.1.351 Strain in a Novel Mouse Model

To further evaluate the protective efficacy of SARS-COV-2 circRNA^{RBD-501Y.V2} vaccines in vivo, the B.1.351/501Y.V2 strain is used for authentic virus challenge experiments due to its severe antibody escape capability. Consistent with a recent report, the B.1.351/501Y.V2 variant could infect BALB/c mice and replicate in their lungs, possibly due to the mutations in spike protein, especially in the RBD domain, such as K417N, E484K and N501Y. BALB/c mice is used for accessing the protective efficacy of SARS-COV-2 circRNA^{RBD-501Y.V2} vaccines. BALB/c mice are administered with two-dose immunization of 50 μg circRNA^{RBD-501Y.V2}, vaccine or placebo via the i.m. route, at a two-week interval. To evaluate the long-term protection of circRNA vaccines, each immunized mouse is challenged with 5×10⁴ PFU of authentic SARS-COV-2 B.1.351/501Y.V2 strain via the intranasal (i.n.) route at 7 weeks post the boost dose, and the lung tissues are collected 3 days after challenge for detecting viral RNA. Three days before virus challenge, the sera of immunized mice are collected to detect the RBD-501Y.V2-specific IgG. The titer of RBD-501Y.V2-specific IgG is expected to be high long after immunization (e.g., two months later), and the serum is expected to show significant neutralizing capacity against RBD-501Y.V2 antigens.

Mice in the placebo group may also undergo weight loss compared with vaccinated mice. Virus titers in the lungs of vaccinated mice are expected to significantly decrease compared with those who received placebos. These results may indicate that the circRNA^{RBD-501Y.V2} vaccine can effectively protect the mice from infection by SARS-COV-2 B.1.351/501Y.V2 variants.

### Example 15. Luciferase Expression by Naked circRNA^{Luc} in Mice

Group I ribozyme autocatalysis strategies were adopted to generate circRNA^{Luc} encoding firefly luciferase (Luc). The circRNA^{Luc} was then diluted in PBS. Mice were injected intramuscularly with PBS or 20 μg of naked circRNA^{Luc}, while untreated group served as mock control. 24 hrs post injection, mice were injected intraperitoneally with luciferin and transferred to the IVIS imaging system. Naked circRNA Luc showed local expression of firefly luciferase in the injection site (FIGS. 9A-9C)

### Example 16. Naked circRNA Vaccines Elicited SARS-COV-2 RBD-Specific Antibodies in Mice

Firstly, Group I ribozyme autocatalysis strategies were adopted to generate circRNA^{RBD-Beta} vaccine comprising naked circRNA^{RBD-Beta} encoding RBD antigens of Beta variant without LNP encapsulation. The circRNA^{RBD-Beta} was then diluted in PBS. Mice were immunized i.m. with 2 doses of 100 μg circRNA^{RBD-Beta} vaccine twice at a 2-week interval and a third dose of 250 μg circRNA^{RBD-Beta} vaccine at 3 weeks post boost. PBS were used as the placebo control. 2 weeks post the third boost, the serum samples from immunized mice were collected for the detection of RBD specific antibodies (FIG. 10A). CircRNA RBD-Beta vaccines comprising naked circRNA^{RBD-Beta} induced RBD-specific IgG binding antibodies in mice (FIG. 10B).

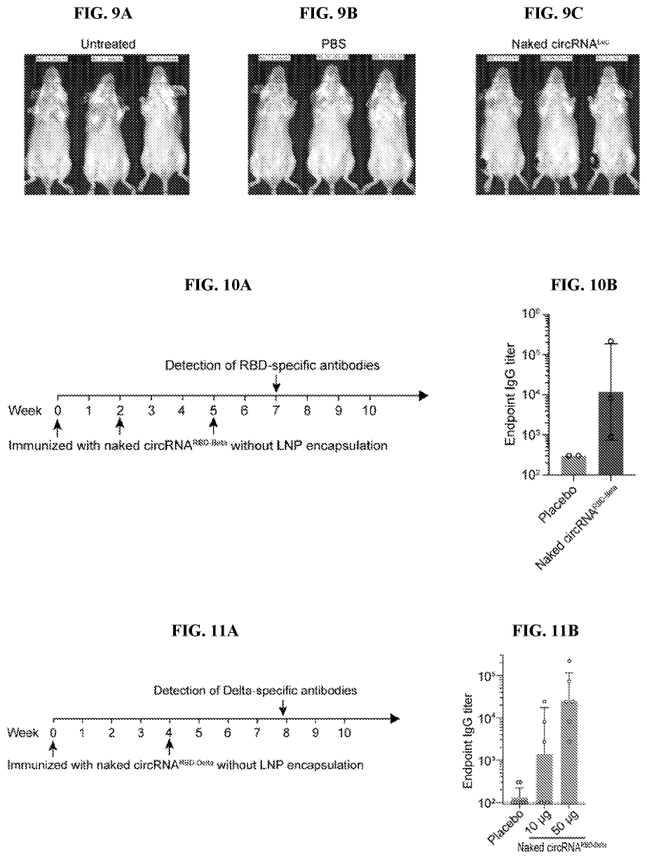

In consideration that RBD-Delta has more Immunogenicity and LNP-encapsulated circRNA^{RBD-Delta} vaccine could induce higher IgG titer in mice than LNP-encapsulated circRNA^{RBD-Beta} vaccine, Group I ribozyme autocatalysis strategies were adopted to generate circRNA^{RBD-Delta} vaccine comprising naked circRNA^{RBD-Delta} encoding RBD antigens of Delta variant without LNP encapsulation. The circRNA^{RBD-Delta} was then diluted in PBS. Mice were immunized i.m. with 10 or 50 μg of circRNA^{RBD-Delta} vaccine twice at a 4-week interval, while PBS were used as the placebo control. 25 days post boost, the serum samples from immunized mice were collected for the detection of RBD-Delta specific antibodies (FIG. 11A). CircRNA^{RBD-Delta} vaccines induced RBD-Delta specific IgG binding antibodies in mice, while 50 μg administration group generated higher IgG level than 10 μg administration group (FIG. 11B).

### Example 17. Naked circRNA Vaccines Elicited Higher Antibodies than mRNA Vaccines in Mice

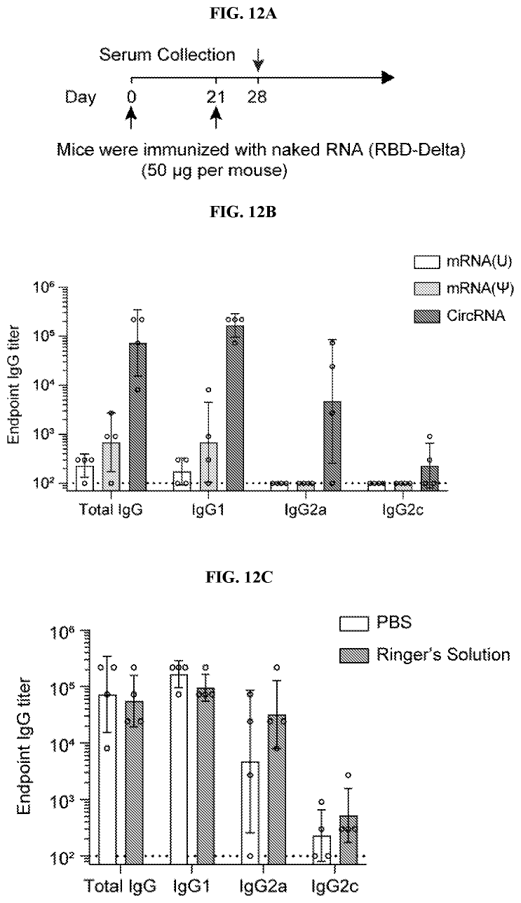

Group I ribozyme autocatalysis strategies were adopted to generate naked circRNA^{RBD-Delta} vaccine encoding RBD antigens of Delta variant without LNP encapsulation. For naked mRNA^{RBD-Delta} vaccines, modified or unmodified mRNA was generated with or without 1m-modified UTP. RNA vaccines were then diluted in PBS. Mice were immunized 50 μg of naked RNA vaccines twice at a 3-week interval. 7 days post boost, the serum samples from immunized mice were collected for the detection of RBD-Delta specific antibodies, including total IgG, IgG1, IgG2a and IgG2c (FIG. 12A). Naked circRNA^{RBD-Delta} vaccines induced potent antibodies in mice, while naked modified or unmodified mRNA^{RBD-Delta} vaccines did not induce potent antibodies in mice (FIG. 12B). Besides, in consideration that Ringer's solution was used in naked mRNA vaccines before, the difference between PBS and Ringer's solution as the buffer of naked circRNA was compared. Using Ringer's solution, circRNA vaccine induced higher IgG2a and IgG2c, suggesting higher quality of antibodies. (FIG. 12C).

The examples set forth above are provided to give those of ordinary skill in the art a complete disclosure and description of how to make and use the embodiments of the circRNA, vaccines, compositions, and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of the artisans to which the invention pertains.

Many modifications and variations of this application can be made without departing from its spirit and scope, as will be apparent to those skilled in the art. The specific embodiments and examples described herein are offered by way of example only.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

EXEMPLARY SEQUENCES

SEQ ID NO: 1 Full-length S protein sequence of SARS-CoV-2
MFVFLVLLPLVSSQCVNLTTRTQLPPAYTNSFTRGVYYPDKVFRSSVLHSTQDLFLPFFSNVTWFHAIHVSG
TNGTKRFDNPVLPFNDGVYFASTEKSNIIRGWIFGTTLDSKTQSLLIVNNATNVVIKVCEFQFCNDPFLGVY
YHKNNKSWMESEFRVYSSANNCTFEYVSQPFLMDLEGKQGNFKNLREFVFKNIDGYFKIYSKHTPINLVRD
LPQGFSALEPLVDLPIGINITRFQTLLALHRSYLTPGDSSSGWTAGAAAYYVGYLQPRTFLLKYNENGTITD
AVDCALDPLSETKCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIADYNYKLPDDFTG
CVIAWNSNNLDSKVGGNYNYLYRLFRKSNLKPFERDISTEIYQAGSTPCNGVEGFNCYFPLQSYGFQPTNG
VGYQPYRVVVLSFELLHAPATVCGPKKSTNLVKNKCVNFNFNGLTGTGVLTESNKKFLPFQQFGRDIADTT
DAVRDPQTLEILDITPCSFGGVSVITPGTNTSNQVAVLYQDVNCTEVPVAIHADQLTPTWRVYSTGSNVFQT
RAGCLIGAEHVNNSYECDIPIGAGICASYQTQTNSPRRARSVASQSIIAYTMSLGAENSVAYSNNSIAIPTNFT
ISVTTEILPVSMTKTSVDCTMYICGDSTECSNLLLQYGSFCTQLNRALTGIAVEQDKNTQEVFAQVKQIYKT
PPIKDFGGFNFSQILPDPSKPSKRSFIEDLLFNKVTLADAGFIKQYGDCLGDIAARDLICAQKFNGLTVLPPLL
TDEMIAQYTSALLAGTITSGWTFGAGAALQIPFAMQMAYRFNGIGVTQNVLYENQKLIANQFNSAIGKIQD
SLSSTASALGKLQDVVNQNAQALNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDLQELGKYEQYIKWPWYIWLGFIA
GLIAIVMVTIMLCCMTSCCSCLKGCCSCGSCCKFDEDDSEPVLKGVKLHYT
SEQ ID NO: 2 RBD amino acid residues 319-542 of S protein
RVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELLHAPATVCGPKKSTN
LVKNKCVNF
SEQ ID NO: 3 C-terminal Foldon domain of a T4 fibritin domain
GSGYIPEAPRDGQAYVRKDGEWVLLSTFLGRS
SEQ ID NO: 4 GCN4-based leucine zipper domain
RMKQIEDKIEEILSKIYHIENEIARIKKLIGER
SEQ ID NO: 5 Exemplary peptide linker
GGGGSGGGGS
SEQ ID NO: 6 wildtype S2 region of SARS-CoV-2 S protein
SVASQSIIAYTMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLVKQLSSNFGAISS
VLNDILSRLDKVEAEVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWFVTQRNFYEPQIITT
DNTFVSGNCDVVIGIVNNTVYDPLQPELDSFKEELDKYFKNHTSPDVDLGDISGINASVVNIQKEIDRLNEV
AKNLNESLIDLQELGKYEQYIKWPWYIWLGFIAGLIAIVMVTIMLCCMTSCCSCLKGCCSCGSCCKFDEDD
SEPVLKGVKLHYT
SEQ ID NO: 7 K986P/V987P S2 region sequence of SARS-CoV-2 S protein
SVASQSIIAYTMSLGAENSVAYSNNSIAIPTNFTISVTTEILPVSMTKTSVDCTMYICGDSTECSNLLLQYGSF
CTQLNRALTGIAVEQDKNTQEVFAQVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLVKQLSSNFGAISS
VLNDILSRLDPPEAEVQIDRLITGRLQSLQTYVTQQLIRAAEIRASANLAATKMSECVLGQSKRVDFCGKGY
HLMSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNEVA
KNLNESLIDLQELGKYEQYIKWPWYIWLGFIAGLIAIVMVTIMLCCMTSCCSCLKGCCSCGSCCKFDEDDSE
PVLKGVKLHYT
SEQ ID NO: 8 wildtype amino acid residues 2-1273 sequence of S protein of SARS-CoV-2
FVFLVLLPLVSSQCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFKNIDGYFKIYSKHTPINLVRDL
PQGFSALEPLVDLPIGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPTWRVYSTGSNVFQTR
AGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFNSAIGKIQDS
LSSTASALGKLQDVVNQNAQALNTLVKQLSSNFGAISSVLNDILSRLDKVEAEVQIDRLITGRLQSLQTYVT
QQLIRAAEIRASANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHYT
SEQ ID NO: 9 amino acid residues 2-1273 sequence of S protein of SARS-CoV-2, Δ681-684
FVFLVLLPLVSSQCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFKNIDGYFKIYSKHTPINLVRDL
PQGFSALEPLVDLPIGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPTWRVYSTGSNVFQTR
AGCLIGAEHVNNSYECDIPIGAGICASYQTQTNSRSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAARDLICAQKFNGLTVLPPLLTDEMI
AQYTSALLAGTITSGWTFGAGAALQIPFAMQMAYRFNGIGVTQNVLYENQKLIANQFNSAIGKIQDSLSST
ASALGKLQDVVNQNAQALNTLVKQLSSNFGAISSVLNDILSRLDKVEAEVQIDRLITGRLQSLQTYVTQQLI
RAAEIRASANLAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
SEQ ID NO: 10 amino acid residues 2-1273 sequence of S protein of SARS-CoV-2, K986P V987P Δ681-684
sequence
FVFLVLLPLVSSQCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFKNIDGYFKIYSKHTPINLVRDL
PQGFSALEPLVDLPIGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPTWRVYSTGSNVFQTR
AGCLIGAEHVNNSYECDIPIGAGICASYQTQTNSRSVASQSIIAYTMSLGAENSVAYSNNSIAIPTNFTISVTT
EILPVSMTKTSVDCTMYICGDSTECSNLLLQYGSFCTQLNRALTGIAVEQDKNTQEVFAQVKQIYKTPPIKD
FGGFNFSQILPDPSKPSKRSFIEDLLFNKVTLADAGFIKQYGDCLGDIAARDLICAQKFNGLTVLPPLLTDEMI
AQYTSALLAGTITSGWTFGAGAALQIPFAMQMAYRFNGIGVTQNVLYENQKLIANQFNSAIGKIQDSLSST
ASALGKLQDVVNQNAQALNTLVKQLSSNFGAISSVLNDILSRLDPPEAEVQIDRLITGRLQSLQTYVTQQLI
RAAEIRASANLAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
SEQ ID NO: 11 Nucleic acid sequence of the wildtype S2 region sequence
AGTGTGGCTTCTCAAAGCATTATAGCATACACTATGTCTCTTGGTGCCGAAAATTCCGTGGCCTATTCT
AACAATTCAATCGCCATCCCAACCAACTTCACAATTAGCGTGACTACCGAAATACTGCCTGTGAGCAT
GACGAAAACCAGCGTAGACTGCACTATGTATATCTGTGGAGACTCCACTGAGTGCTCCAACCTTCTCC
TGCAGTACGGTAGCTTCTGTACCCAATTGAACCGCGCCCTTACAGGCATCGCTGTTGAGCAAGATAAG
AATACCCAGGAAGTTTTTGCCCAGGTTAAGCAGATATACAAAACACCGCCCATTAAGGACTTCGGAGG
CTTCAACTTCTCTCAGATACTGCCTGACCCCTCCAAGCCATCAAAACGCAGCTTCATTGAGGACCTCTT
GTTCAACAAAGTGACTCTGGCTGATGCTGGCTTCATTAAGCAGTACGGAGATTGCCTGGGAGATATTG
CTGCCAGGGACCTCATCTGCGCCCAGAAGTTTAATGGCCTGACAGTCTTGCCCCCACTTCTGACAGAC
GAGATGATTGCTCAGTACACATCTGCCCTCCTCGCTGGCACCATAACATCCGGATGGACATTTGGTGCT
GGTGCTGCCCTCCAGATTCCCTTCGCAATGCAGATGGCGTATCGCTTTAACGGCATCGGTGTCACACAA
AACGTGTTGTATGAGAACCAAAAGCTCATCGCTAACCAGTTTAATTCTGCTATTGGTAAGATTCAGGA
CAGCCTGTCATCAACCGCGTCTGCCCTTGGTAAGTTGCAGGACGTGGTGAACCAGAATGCTCAGGCTT
TGAATACTCTGGTGAAGCAACTCTCTTCAAATTTCGGCGCTATCTCTTCTGTGTTGAACGACATCCTGA
GTCGCCTTGATAAGGTGGAAGCTGAAGTTCAAATTGATAGATTGATTACTGGCAGGCTCCAGTCTTTG
CAGACCTACGTTACACAGCAGCTGATTAGGGCGGCTGAAATTAGAGCTTCCGCCAATCTGGCTGCAAC
CAAGATGTCCGAATGCGTCCTGGGTCAGTCAAAGCGCGTTGACTTTTGTGGTAAAGGCTACCACCTCA
TGTCATTTCCCCAGTCAGCACCTCACGGAGTAGTGTTCCTCCACGTCACCTACGTTCCAGCACAGGAAA
AGAATTTTACCACTGCGCCGGCAATCTGTCACGACGGTAAGGCACACTTCCCCCGCGAGGGCGTATTC
GTGTCTAACGGAACTCATTGGTTCGTCACACAGAGAAACTTCTATGAGCCTCAGATCATTACCACCGA
CAATACATTTGTGTCCGGTAACTGCGACGTTGTGATTGGAATCGTCAACAACACTGTGTACGATCCACT
TCAGCCAGAACTGGATAGCTTCAAGGAAGAATTGGACAAATATTTCAAAAATCACACTTCACCCGATG
TGGACCTGGGTGACATTAGTGGTATCAATGCGTCCGTGGTCAATATTCAAAAAGAGATTGACAGGCTC
AACGAAGTGGCCAAGAACCTGAACGAAAGTCTTATCGATCTGCAAGAATTGGGAAAGTATGAGCAGT
ACATCAAGTGGCCGTGGTACATTTGGTTGGGTTTTATCGCCGGTCTGATCGCCATCGTTATGGTTACCA
TTATGCTTTGCTGCATGACGAGCTGTTGCTCCTGTCTGAAGGGATGCTGCTCTTGCGGATCATGTTGCA
AGTTCGATGAAGACGATAGCGAACCAGTTCTGAAGGGCGTCAAGCTGCATTACACA
SEQ ID NO: 12 Nucleic acid sequence of the K986P/V987P S2 region sequence
AGTGTGGCTTCTCAAAGCATTATAGCATACACTATGTCTCTTGGTGCCGAAAATTCCGTGGCCTATTCT
AACAATTCAATCGCCATCCCAACCAACTTCACAATTAGCGTGACTACCGAAATACTGCCTGTGAGCAT
GACGAAAACCAGCGTAGACTGCACTATGTATATCTGTGGAGACTCCACTGAGTGCTCCAACCTTCTCC
TGCAGTACGGTAGCTTCTGTACCCAATTGAACCGCGCCCTTACAGGCATCGCTGTTGAGCAAGATAAG
AATACCCAGGAAGTTTTTGCCCAGGTTAAGCAGATATACAAAACACCGCCCATTAAGGACTTCGGAGG
CTTCAACTTCTCTCAGATACTGCCTGACCCCTCCAAGCCATCAAAACGCAGCTTCATTGAGGACCTCTT
GTTCAACAAAGTGACTCTGGCTGATGCTGGCTTCATTAAGCAGTACGGAGATTGCCTGGGAGATATTG
CTGCCAGGGACCTCATCTGCGCCCAGAAGTTTAATGGCCTGACAGTCTTGCCCCCACTTCTGACAGAC
GAGATGATTGCTCAGTACACATCTGCCCTCCTCGCTGGCACCATAACATCCGGATGGACATTTGGTGCT
GGTGCTGCCCTCCAGATTCCCTTCGCAATGCAGATGGCGTATCGCTTTAACGGCATCGGTGTCACACAA
AACGTGTTGTATGAGAACCAAAAGCTCATCGCTAACCAGTTTAATTCTGCTATTGGTAAGATTCAGGA
CAGCCTGTCATCAACCGCGTCTGCCCTTGGTAAGTTGCAGGACGTGGTGAACCAGAATGCTCAGGCTT
TGAATACTCTGGTGAAGCAACTCTCTTCAAATTTCGGCGCTATCTCTTCTGTGTTGAACGACATCCTGA
GTCGCCTTGATcctccaGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCTGCAACCAA
GATGTCCGAATGCGTCCTGGGTCAGTCAAAGCGCGTTGACTTTTGTGGTAAAGGCTACCACCTCATGTC
ATTTCCCCAGTCAGCACCTCACGGAGTAGTGTTCCTCCACGTCACCTACGTTCCAGCACAGGAAAAGA
ATTTTACCACTGCGCCGGCAATCTGTCACGACGGTAAGGCACACTTCCCCCGCGAGGGCGTATTCGTG
TCTAACGGAACTCATTGGTTCGTCACACAGAGAAACTTCTATGAGCCTCAGATCATTACCACCGACAA
TACATTTGTGTCCGGTAACTGCGACGTTGTGATTGGAATCGTCAACAACACTGTGTACGATCCACTTCA
GCCAGAACTGGATAGCTTCAAGGAAGAATTGGACAAATATTTCAAAAATCACACTTCACCCGATGTGG
ACCTGGGTGACATTAGTGGTATCAATGCGTCCGTGGTCAATATTCAAAAAGAGATTGACAGGCTCAAC
GAAGTGGCCAAGAACCTGAACGAAAGTCTTATCGATCTGCAAGAATTGGGAAAGTATGAGCAGTACA
TCAAGTGGCCGTGGTACATTTGGTTGGGTTTTATCGCCGGTCTGATCGCCATCGTTATGGTTACCATTA
TGCTTTGCTGCATGACGAGCTGTTGCTCCTGTCTGAAGGGATGCTGCTCTTGCGGATCATGTTGCAAGT
TCGATGAAGACGATAGCGAACCAGTTCTGAAGGGCGTCAAGCTGCATTACACA
SEQ ID NO: 13 Nucleic acid sequence of the wildtype 2-1273 sequence of Spike
TTCGTTTTCCTTGTTCTGTTGCCTCTCGTTAGTAGCCAATGCGTCAACCTTACTACTAGAACCCAGCTCC
CTCCAGCATATACCAACTCTTTCACCAGGGGCGTATATTACCCGGACAAAGTGTTCCGCTCAAGTGTGC
TGCATTCTACGCAGGACCTTTTCTTGCCCTTTTTCAGTAATGTTACTTGGTTTCATGCTATCCATGTGTC
TGGAACTAACGGAACCAAGCGCTTTGACAACCCCGTCCTCCCTTTCAACGATGGCGTGTACTTCGCTTC
CACGGAAAAGTCAAACATAATTCGCGGCTGGATCTTTGGTACAACACTCGACTCAAAGACGCAGAGCC
TGCTGATCGTTAATAACGCTACAAATGTTGTGATAAAGGTGTGTGAATTTCAGTTCTGCAATGATCCCT
TCCTGGGTGTGTACTACCATAAGAATAACAAGAGCTGGATGGAATCCGAATTTAGGGTTTACAGTTCC
GCTAACAACTGCACATTCGAATACGTAAGCCAGCCATTTCTTATGGATCTTGAGGGCAAGCAAGGAAA
CTTCAAGAACTTGAGGGAGTTCGTGTTCAAAAATATCGACGGCTATTTTAAGATATATAGCAAGCACA
CTCCAATAAACTTGGTGCGCGACCTGCCCCAGGGATTCTCTGCTCTGGAGCCCCTGGTGGATCTGCCCA
TTGGAATAAACATAACTCGCTTTCAAACACTGCTCGCCCTGCATCGCAGTTACCTCACCCCTGGTGATA
GTAGTTCAGGATGGACAGCAGGAGCCGCCGCATACTACGTCGGCTACCTGCAGCCTAGGACCTTCTTG
CTGAAGTACAACGAGAACGGTACAATAACTGACGCTGTGGACTGCGCTCTGGACCCTCTGTCCGAGAC
GAAGTGCACCCTGAAGAGCTTTACTGTTGAAAAAGGCATTTACCAAACCAGCAACTTCCGCGTCCAGC
CAACCGAGAGCATCGTCAGATTTCCCAACATTACAAATCTGTGTCCCTTCGGCGAGGTGTTCAACGCC
ACACGCTTCGCTTCAGTGTACGCATGGAACCGCAAGCGCATATCTAACTGCGTCGCGGATTATTCTGTC
CTCTACAACTCCGCCTCTTTCTCCACCTTCAAGTGCTACGGAGTGTCACCGACTAAGCTGAACGATCTC
TGCTTTACCAACGTCTACGCGGACTCCTTCGTGATAAGAGGTGATGAAGTGAGACAAATAGCCCCAGG
TCAGACTGGTAAGATCGCAGATTACAACTACAAATTGCCTGATGATTTCACTGGTTGCGTTATCGCGTG
GAACTCTAATAACCTCGATTCTAAGGTCGGTGGTAACTACAATTACCTGTACCGCTTGTTTAGGAAGTC
AAACCTGAAGCCTTTCGAGAGGGATATTTCAACCGAAATCTATCAAGCGGGTTCAACACCGTGTAACG
GTGTGGAAGGATTTAACTGCTACTTCCCCCTGCAGTCTTACGGATTCCAGCCAACCAATGGCGTGGGTT
ACCAACCTTATCGCGTGGTGGTTCTGAGTTTCGAACTGTTGCACGCTCCCGCCACGGTATGCGGTCCCA
AGAAGAGCACTAACTTGGTGAAGAATAAGTGCGTGAATTTCAATTTCAATGGCCTCACTGGAACTGGA
GTGCTGACCGAATCCAATAAGAAGTTCTTGCCCTTCCAGCAGTTCGGAAGAGACATTGCTGACACAAC
CGACGCGGTGCGCGATCCTCAGACTCTGGAGATATTGGACATTACACCATGTTCTTTCGGCGGTGTGTC
TGTCATTACTCCGGGCACGAATACTAGCAACCAGGTAGCCGTGCTGTACCAAGACGTGAATTGCACAG
AGGTTCCCGTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAGTGCGACAT
CCCCATCGGAGCCGGTATATGCGCCTCTTATCAGACACAAACTAACTCACCCAGGAGAGCCCGCAGTG
TGGCTTCTCAAAGCATTATAGCATACACTATGTCTCTTGGTGCCGAAAATTCCGTGGCCTATTCTAACA
ATTCAATCGCCATCCCAACCAACTTCACAATTAGCGTGACTACCGAAATACTGCCTGTGAGCATGACG
AAAACCAGCGTAGACTGCACTATGTATATCTGTGGAGACTCCACTGAGTGCTCCAACCTTCTCCTGCA
GTACGGTAGCTTCTGTACCCAATTGAACCGCGCCCTTACAGGCATCGCTGTTGAGCAAGATAAGAATA
CCCAGGAAGTTTTTGCCCAGGTTAAGCAGATATACAAAACACCGCCCATTAAGGACTTCGGAGGCTTC
AACTTCTCTCAGATACTGCCTGACCCCTCCAAGCCATCAAAACGCAGCTTCATTGAGGACCTCTTGTTC
AACAAAGTGACTCTGGCTGATGCTGGCTTCATTAAGCAGTACGGAGATTGCCTGGGAGATATTGCTGC
CAGGGACCTCATCTGCGCCCAGAAGTTTAATGGCCTGACAGTCTTGCCCCCACTTCTGACAGACGAGA
TGATTGCTCAGTACACATCTGCCCTCCTCGCTGGCACCATAACATCCGGATGGACATTTGGTGCTGGTG
CTGCCCTCCAGATTCCCTTCGCAATGCAGATGGCGTATCGCTTTAACGGCATCGGTGTCACACAAAAC
GTGTTGTATGAGAACCAAAAGCTCATCGCTAACCAGTTTAATTCTGCTATTGGTAAGATTCAGGACAG
CCTGTCATCAACCGCGTCTGCCCTTGGTAAGTTGCAGGACGTGGTGAACCAGAATGCTCAGGCTTTGA
ATACTCTGGTGAAGCAACTCTCTTCAAATTTCGGCGCTATCTCTTCTGTGTTGAACGACATCCTGAGTC
GCCTTGATAAGGTGGAAGCTGAAGTTCAAATTGATAGATTGATTACTGGCAGGCTCCAGTCTTTGCAG
ACCTACGTTACACAGCAGCTGATTAGGGCGGCTGAAATTAGAGCTTCCGCCAATCTGGCTGCAACCAA
GATGTCCGAATGCGTCCTGGGTCAGTCAAAGCGCGTTGACTTTTGTGGTAAAGGCTACCACCTCATGTC
ATTTCCCCAGTCAGCACCTCACGGAGTAGTGTTCCTCCACGTCACCTACGTTCCAGCACAGGAAAAGA
ATTTTACCACTGCGCCGGCAATCTGTCACGACGGTAAGGCACACTTCCCCCGCGAGGGCGTATTCGTG
TCTAACGGAACTCATTGGTTCGTCACACAGAGAAACTTCTATGAGCCTCAGATCATTACCACCGACAA
TACATTTGTGTCCGGTAACTGCGACGTTGTGATTGGAATCGTCAACAACACTGTGTACGATCCACTTCA
GCCAGAACTGGATAGCTTCAAGGAAGAATTGGACAAATATTTCAAAAATCACACTTCACCCGATGTGG
ACCTGGGTGACATTAGTGGTATCAATGCGTCCGTGGTCAATATTCAAAAAGAGATTGACAGGCTCAAC
GAAGTGGCCAAGAACCTGAACGAAAGTCTTATCGATCTGCAAGAATTGGGAAAGTATGAGCAGTACA
TCAAGTGGCCGTGGTACATTTGGTTGGGTTTTATCGCCGGTCTGATCGCCATCGTTATGGTTACCATTA
TGCTTTGCTGCATGACGAGCTGTTGCTCCTGTCTGAAGGGATGCTGCTCTTGCGGATCATGTTGCAAGT
TCGATGAAGACGATAGCGAACCAGTTCTGAAGGGCGTCAAGCTGCATTACACA
SEQ ID NO: 14 Nucleic acid sequence of the Δ681-684 sequence of Spike
TTCGTTTTCCTTGTTCTGTTGCCTCTCGTTAGTAGCCAATGCGTCAACCTTACTACTAGAACCCAGCTCC
CTCCAGCATATACCAACTCTTTCACCAGGGGCGTATATTACCCGGACAAAGTGTTCCGCTCAAGTGTGC
TGCATTCTACGCAGGACCTTTTCTTGCCCTTTTTCAGTAATGTTACTTGGTTTCATGCTATCCATGTGTC
TGGAACTAACGGAACCAAGCGCTTTGACAACCCCGTCCTCCCTTTCAACGATGGCGTGTACTTCGCTTC
CACGGAAAAGTCAAACATAATTCGCGGCTGGATCTTTGGTACAACACTCGACTCAAAGACGCAGAGCC
TGCTGATCGTTAATAACGCTACAAATGTTGTGATAAAGGTGTGTGAATTTCAGTTCTGCAATGATCCCT
TCCTGGGTGTGTACTACCATAAGAATAACAAGAGCTGGATGGAATCCGAATTTAGGGTTTACAGTTCC
GCTAACAACTGCACATTCGAATACGTAAGCCAGCCATTTCTTATGGATCTTGAGGGCAAGCAAGGAAA
CTTCAAGAACTTGAGGGAGTTCGTGTTCAAAAATATCGACGGCTATTTTAAGATATATAGCAAGCACA
CTCCAATAAACTTGGTGCGCGACCTGCCCCAGGGATTCTCTGCTCTGGAGCCCCTGGTGGATCTGCCCA
TTGGAATAAACATAACTCGCTTTCAAACACTGCTCGCCCTGCATCGCAGTTACCTCACCCCTGGTGATA
GTAGTTCAGGATGGACAGCAGGAGCCGCCGCATACTACGTCGGCTACCTGCAGCCTAGGACCTTCTTG
CTGAAGTACAACGAGAACGGTACAATAACTGACGCTGTGGACTGCGCTCTGGACCCTCTGTCCGAGAC
GAAGTGCACCCTGAAGAGCTTTACTGTTGAAAAAGGCATTTACCAAACCAGCAACTTCCGCGTCCAGC
CAACCGAGAGCATCGTCAGATTTCCCAACATTACAAATCTGTGTCCCTTCGGCGAGGTGTTCAACGCC
ACACGCTTCGCTTCAGTGTACGCATGGAACCGCAAGCGCATATCTAACTGCGTCGCGGATTATTCTGTC
CTCTACAACTCCGCCTCTTTCTCCACCTTCAAGTGCTACGGAGTGTCACCGACTAAGCTGAACGATCTC
TGCTTTACCAACGTCTACGCGGACTCCTTCGTGATAAGAGGTGATGAAGTGAGACAAATAGCCCCAGG
TCAGACTGGTAAGATCGCAGATTACAACTACAAATTGCCTGATGATTTCACTGGTTGCGTTATCGCGTG
GAACTCTAATAACCTCGATTCTAAGGTCGGTGGTAACTACAATTACCTGTACCGCTTGTTTAGGAAGTC
AAACCTGAAGCCTTTCGAGAGGGATATTTCAACCGAAATCTATCAAGCGGGTTCAACACCGTGTAACG
GTGTGGAAGGATTTAACTGCTACTTCCCCCTGCAGTCTTACGGATTCCAGCCAACCAATGGCGTGGGTT
ACCAACCTTATCGCGTGGTGGTTCTGAGTTTCGAACTGTTGCACGCTCCCGCCACGGTATGCGGTCCCA
AGAAGAGCACTAACTTGGTGAAGAATAAGTGCGTGAATTTCAATTTCAATGGCCTCACTGGAACTGGA
GTGCTGACCGAATCCAATAAGAAGTTCTTGCCCTTCCAGCAGTTCGGAAGAGACATTGCTGACACAAC
CGACGCGGTGCGCGATCCTCAGACTCTGGAGATATTGGACATTACACCATGTTCTTTCGGCGGTGTGTC
TGTCATTACTCCGGGCACGAATACTAGCAACCAGGTAGCCGTGCTGTACCAAGACGTGAATTGCACAG
AGGTTCCCGTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAGTGCGACAT
CCCCATCGGAGCCGGTATATGCGCCTCTTATCAGACACAAACTAACTCACGCAGTGTGGCTTCTCAAA
GCATTATAGCATACACTATGTCTCTTGGTGCCGAAAATTCCGTGGCCTATTCTAACAATTCAATCGCCA
TCCCAACCAACTTCACAATTAGCGTGACTACCGAAATACTGCCTGTGAGCATGACGAAAACCAGCGTA
GACTGCACTATGTATATCTGTGGAGACTCCACTGAGTGCTCCAACCTTCTCCTGCAGTACGGTAGCTTC
TGTACCCAATTGAACCGCGCCCTTACAGGCATCGCTGTTGAGCAAGATAAGAATACCCAGGAAGTTTT
TGCCCAGGTTAAGCAGATATACAAAACACCGCCCATTAAGGACTTCGGAGGCTTCAACTTCTCTCAGA
TACTGCCTGACCCCTCCAAGCCATCAAAACGCAGCTTCATTGAGGACCTCTTGTTCAACAAAGTGACTC
TGGCTGATGCTGGCTTCATTAAGCAGTACGGAGATTGCCTGGGAGATATTGCTGCCAGGGACCTCATC
TGCGCCCAGAAGTTTAATGGCCTGACAGTCTTGCCCCCACTTCTGACAGACGAGATGATTGCTCAGTA
CACATCTGCCCTCCTCGCTGGCACCATAACATCCGGATGGACATTTGGTGCTGGTGCTGCCCTCCAGAT
TCCCTTCGCAATGCAGATGGCGTATCGCTTTAACGGCATCGGTGTCACACAAAACGTGTTGTATGAGA
ACCAAAAGCTCATCGCTAACCAGTTTAATTCTGCTATTGGTAAGATTCAGGACAGCCTGTCATCAACC
GCGTCTGCCCTTGGTAAGTTGCAGGACGTGGTGAACCAGAATGCTCAGGCTTTGAATACTCTGGTGAA
GCAACTCTCTTCAAATTTCGGCGCTATCTCTTCTGTGTTGAACGACATCCTGAGTCGCCTTGATAAGGT
GGAAGCTGAAGTTCAAATTGATAGATTGATTACTGGCAGGCTCCAGTCTTTGCAGACCTACGTTACAC
AGCAGCTGATTAGGGCGGCTGAAATTAGAGCTTCCGCCAATCTGGCTGCAACCAAGATGTCCGAATGC
GTCCTGGGTCAGTCAAAGCGCGTTGACTTTTGTGGTAAAGGCTACCACCTCATGTCATTTCCCCAGTCA
GCACCTCACGGAGTAGTGTTCCTCCACGTCACCTACGTTCCAGCACAGGAAAAGAATTTTACCACTGC
GCCGGCAATCTGTCACGACGGTAAGGCACACTTCCCCCGCGAGGGCGTATTCGTGTCTAACGGAACTC
ATTGGTTCGTCACACAGAGAAACTTCTATGAGCCTCAGATCATTACCACCGACAATACATTTGTGTCCG
GTAACTGCGACGTTGTGATTGGAATCGTCAACAACACTGTGTACGATCCACTTCAGCCAGAACTGGAT
AGCTTCAAGGAAGAATTGGACAAATATTTCAAAAATCACACTTCACCCGATGTGGACCTGGGTGACAT
TAGTGGTATCAATGCGTCCGTGGTCAATATTCAAAAAGAGATTGACAGGCTCAACGAAGTGGCCAAGA
ACCTGAACGAAAGTCTTATCGATCTGCAAGAATTGGGAAAGTATGAGCAGTACATCAAGTGGCCGTGG
TACATTTGGTTGGGTTTTATCGCCGGTCTGATCGCCATCGTTATGGTTACCATTATGCTTTGCTGCATGA
CGAGCTGTTGCTCCTGTCTGAAGGGATGCTGCTCTTGCGGATCATGTTGCAAGTTCGATGAAGACGAT
AGCGAACCAGTTCTGAAGGGCGTCAAGCTGCATTACACA
SEQ ID NO: 15 Nucleic acid sequence of the K986P/V987P Δ681-684 sequence of Spike
TTCGTTTTCCTTGTTCTGTTGCCTCTCGTTAGTAGCCAATGCGTCAACCTTACTACTAGAACCCAGCTCC
CTCCAGCATATACCAACTCTTTCACCAGGGGCGTATATTACCCGGACAAAGTGTTCCGCTCAAGTGTGC
TGCATTCTACGCAGGACCTTTTCTTGCCCTTTTTCAGTAATGTTACTTGGTTTCATGCTATCCATGTGTC
TGGAACTAACGGAACCAAGCGCTTTGACAACCCCGTCCTCCCTTTCAACGATGGCGTGTACTTCGCTTC
CACGGAAAAGTCAAACATAATTCGCGGCTGGATCTTTGGTACAACACTCGACTCAAAGACGCAGAGCC
TGCTGATCGTTAATAACGCTACAAATGTTGTGATAAAGGTGTGTGAATTTCAGTTCTGCAATGATCCCT
TCCTGGGTGTGTACTACCATAAGAATAACAAGAGCTGGATGGAATCCGAATTTAGGGTTTACAGTTCC
GCTAACAACTGCACATTCGAATACGTAAGCCAGCCATTTCTTATGGATCTTGAGGGCAAGCAAGGAAA
CTTCAAGAACTTGAGGGAGTTCGTGTTCAAAAATATCGACGGCTATTTTAAGATATATAGCAAGCACA
CTCCAATAAACTTGGTGCGCGACCTGCCCCAGGGATTCTCTGCTCTGGAGCCCCTGGTGGATCTGCCCA
TTGGAATAAACATAACTCGCTTTCAAACACTGCTCGCCCTGCATCGCAGTTACCTCACCCCTGGTGATA
GTAGTTCAGGATGGACAGCAGGAGCCGCCGCATACTACGTCGGCTACCTGCAGCCTAGGACCTTCTTG
CTGAAGTACAACGAGAACGGTACAATAACTGACGCTGTGGACTGCGCTCTGGACCCTCTGTCCGAGAC
GAAGTGCACCCTGAAGAGCTTTACTGTTGAAAAAGGCATTTACCAAACCAGCAACTTCCGCGTCCAGC
CAACCGAGAGCATCGTCAGATTTCCCAACATTACAAATCTGTGTCCCTTCGGCGAGGTGTTCAACGCC
ACACGCTTCGCTTCAGTGTACGCATGGAACCGCAAGCGCATATCTAACTGCGTCGCGGATTATTCTGTC
CTCTACAACTCCGCCTCTTTCTCCACCTTCAAGTGCTACGGAGTGTCACCGACTAAGCTGAACGATCTC
TGCTTTACCAACGTCTACGCGGACTCCTTCGTGATAAGAGGTGATGAAGTGAGACAAATAGCCCCAGG
TCAGACTGGTAAGATCGCAGATTACAACTACAAATTGCCTGATGATTTCACTGGTTGCGTTATCGCGTG
GAACTCTAATAACCTCGATTCTAAGGTCGGTGGTAACTACAATTACCTGTACCGCTTGTTTAGGAAGTC
AAACCTGAAGCCTTTCGAGAGGGATATTTCAACCGAAATCTATCAAGCGGGTTCAACACCGTGTAACG
GTGTGGAAGGATTTAACTGCTACTTCCCCCTGCAGTCTTACGGATTCCAGCCAACCAATGGCGTGGGTT
ACCAACCTTATCGCGTGGTGGTTCTGAGTTTCGAACTGTTGCACGCTCCCGCCACGGTATGCGGTCCCA
AGAAGAGCACTAACTTGGTGAAGAATAAGTGCGTGAATTTCAATTTCAATGGCCTCACTGGAACTGGA
GTGCTGACCGAATCCAATAAGAAGTTCTTGCCCTTCCAGCAGTTCGGAAGAGACATTGCTGACACAAC
CGACGCGGTGCGCGATCCTCAGACTCTGGAGATATTGGACATTACACCATGTTCTTTCGGCGGTGTGTC
TGTCATTACTCCGGGCACGAATACTAGCAACCAGGTAGCCGTGCTGTACCAAGACGTGAATTGCACAG
AGGTTCCCGTCGCAATTCACGCTGACCAGCTGACCCCCACGTGGAGGGTTTACAGCACTGGTAGTAAC
GTCTTCCAGACGAGAGCCGGTTGCTTGATCGGAGCGGAACATGTGAATAACTCCTACGAGTGCGACAT
CCCCATCGGAGCCGGTATATGCGCCTCTTATCAGACACAAACTAACTCACGCAGTGTGGCTTCTCAAA
GCATTATAGCATACACTATGTCTCTTGGTGCCGAAAATTCCGTGGCCTATTCTAACAATTCAATCGCCA
TCCCAACCAACTTCACAATTAGCGTGACTACCGAAATACTGCCTGTGAGCATGACGAAAACCAGCGTA
GACTGCACTATGTATATCTGTGGAGACTCCACTGAGTGCTCCAACCTTCTCCTGCAGTACGGTAGCTTC
TGTACCCAATTGAACCGCGCCCTTACAGGCATCGCTGTTGAGCAAGATAAGAATACCCAGGAAGTTTT
TGCCCAGGTTAAGCAGATATACAAAACACCGCCCATTAAGGACTTCGGAGGCTTCAACTTCTCTCAGA
TACTGCCTGACCCCTCCAAGCCATCAAAACGCAGCTTCATTGAGGACCTCTTGTTCAACAAAGTGACTC
TGGCTGATGCTGGCTTCATTAAGCAGTACGGAGATTGCCTGGGAGATATTGCTGCCAGGGACCTCATC
TGCGCCCAGAAGTTTAATGGCCTGACAGTCTTGCCCCCACTTCTGACAGACGAGATGATTGCTCAGTA
CACATCTGCCCTCCTCGCTGGCACCATAACATCCGGATGGACATTTGGTGCTGGTGCTGCCCTCCAGAT
TCCCTTCGCAATGCAGATGGCGTATCGCTTTAACGGCATCGGTGTCACACAAAACGTGTTGTATGAGA
ACCAAAAGCTCATCGCTAACCAGTTTAATTCTGCTATTGGTAAGATTCAGGACAGCCTGTCATCAACC
GCGTCTGCCCTTGGTAAGTTGCAGGACGTGGTGAACCAGAATGCTCAGGCTTTGAATACTCTGGTGAA
GCAACTCTCTTCAAATTTCGGCGCTATCTCTTCTGTGTTGAACGACATCCTGAGTCGCCTTGATcctccaGA
AGCTGAAGTTCAAATTGATAGATTGATTACTGGCAGGCTCCAGTCTTTGCAGACCTACGTTACACAGC
AGCTGATTAGGGCGGCTGAAATTAGAGCTTCCGCCAATCTGGCTGCAACCAAGATGTCCGAATGCGTC
CTGGGTCAGTCAAAGCGCGTTGACTTTTGTGGTAAAGGCTACCACCTCATGTCATTTCCCCAGTCAGCA
CCTCACGGAGTAGTGTTCCTCCACGTCACCTACGTTCCAGCACAGGAAAAGAATTTTACCACTGCGCC
GGCAATCTGTCACGACGGTAAGGCACACTTCCCCCGCGAGGGCGTATTCGTGTCTAACGGAACTCATT
GGTTCGTCACACAGAGAAACTTCTATGAGCCTCAGATCATTACCACCGACAATACATTTGTGTCCGGT
AACTGCGACGTTGTGATTGGAATCGTCAACAACACTGTGTACGATCCACTTCAGCCAGAACTGGATAG
CTTCAAGGAAGAATTGGACAAATATTTCAAAAATCACACTTCACCCGATGTGGACCTGGGTGACATTA
GTGGTATCAATGCGTCCGTGGTCAATATTCAAAAAGAGATTGACAGGCTCAACGAAGTGGCCAAGAA
CCTGAACGAAAGTCTTATCGATCTGCAAGAATTGGGAAAGTATGAGCAGTACATCAAGTGGCCGTGGT
ACATTTGGTTGGGTTTTATCGCCGGTCTGATCGCCATCGTTATGGTTACCATTATGCTTTGCTGCATGAC
GAGCTGTTGCTCCTGTCTGAAGGGATGCTGCTCTTGCGGATCATGTTGCAAGTTCGATGAAGACGATA
GCGAACCAGTTCTGAAGGGCGTCAAGCTGCATTACACA
SEQ ID NO: 16 tissue plasminogen activator SP
DAMKRGLCCVLLLCGAVFVSPSQEIHARFRR
SEQ ID NO: 17 human IgE immunoglobulin SP
DWTWILFLVAAATRVHS
SEQ ID NO: 18 amino acid sequence of mouse Alpha-L-iduronidase (IDUA) protein
MLTFFAAFLAAPLALAESPYLVRVDAARPLRPLLPFWRSTGFCPPLPHDQADQYDLSWDQQLNLAYIGAVP
HSGIEQVRIHWLLDLITARKSPGQGLMYNFTHLDAFLDLLMENQLLPGFELMGSPSGYFTDFDDKQQVFEW
KDLVSLLARRYIGRYGLTHVSKWNFETWNEPDHHDFDNVSMTTQGFLNYYDACSEGLRIASPTLKLGGPG
DSFHPLPRSPMCWSLLGHCANGTNFFTGEVGVRLDYISLHKKGAGSSIAILEQEMAVVEQVQQLFPEFKDT
PIYNDEADPLVGWSLPQPWRADVTYAALVVKVIAQHQNLLFANSSSSMRYVLLSNDNAFLSYHPYPFSQR
TLTARFQVNNTHPPHVQLLRKPVLTVMGLMALLDGEQLWAEVSKAGAVLDSNHTVGVLASTHHPEGSAA
AWSTTVLIYTSDDTHAHPNHSIPVTLRLRGVPPGLDLVYIVLYLDNQLSSPYSAWQHMGQPVFPSAEQFRR
MRMVEDPVAEAPRPFPARGRLTLHRKLPVPSLLLVHVCTRPLKPPGQVSRLRALPLTHGQLILVWSDERVG
SKCLWTYEIQFSQKGEEYAPINRRPSTFNLFVFSPDTAVVSGSYRVRALDYWARPGPFSDPVTYLDVPAS
SEQ ID NO: 19 amino acid sequence of human Alpha-L-iduronidase (IDUA) protein
MRPLRPRAALLALLASLLAAPPVAPAEAPHLVHVDAARALWPLRRFWRSTGFCPPLPHSQADQYVLSWDQ
QLNLAYVGAVPHRGIKQVRTHWLLELVTTRGSTGRGLSYNFTHLDGYLDLLRENQLLPGFELMGSASGHF
TDFEDKQQVFEWKDLVSSLARRYIGRYGLAHVSKWNFETWNEPDHHDFDNVSMTMQGFLNYYDACSEG
LRAASPALRLGGPGDSFHTPPRSPLSWGLLRHCHDGTNFFTGEAGVRLDYISLHRKGARSSISILEQEKVVA
QQIRQLFPKFADTPIYNDEADPLVGWSLPQPWRADVTYAAMVVKVIAQHQNLLLANTTSAFPYALLSNDN
AFLSYHPHPFAQRTLTARFQVNNTRPPHVQLLRKPVLTAMGLLALLDEEQLWAEVSQAGTVLDSNHTVGV
LASAHRPQGPADAWRAAVLIYASDDTRAHPNRSVAVTLRLRGVPPGPGLVYVTRYLDNGLCSPDGEWRR
LGRPVFPTAEQFRRMRAAEDPVAAAPRPLPAGGRLTLRPALRLPSLLLVHVCARPEKPPGQVTRLRALPLT
QGQLVLVWSDEHVGSKCLWTYEIQFSQDGKAYTPVSRKPSTFNLFVFSPDTGAVSGSYRVRALDYWARPG
PFSDPVPYLEVPVPRGPPSPGNP
SEQ ID NO: 20 amino acid sequence of mouse Ornithine carbamoyltransferase (OTC) protein
MLSNLRILLNNAALRKGHTSVVRHFWCGKPVQSQVQLKGRDLLTLKNFTGEEIQYMLWLSADLKFRIKQK
GEYLPLLQGKSLGMIFEKRSTRTRLSTETGFALLGGHPSFLTTQDIHLGVNESLTDTARVLSSMTDAVLARV
YKQSDLDTLAKEASIPIVNGLSDLYHPIQILADYLTLQEHYGSLKGLTLSWIGDGNNILHSIMMSAAKFGMH
LQAATPKGYEPDPNIVKLAEQYAKENGTKLSMTNDPLEAARGGNVLITDTWISMGQEDEKKKRLQAFQGY
QVTMKTAKVAASDWTFLHCLPRKPEEVDDEVFYSPRSLVFPEAENRKWTIMAVMVSLLTDYSPVLQKPKF
SEQ ID NO: 21 amino acid sequence of mouse Fumarylacetoacetase (FAH) protein
MSFIPVAEDSDFPIQNLPYGVFSTQSNPKPRIGVAIGDQILDLSVIKHLFTGPALSKHQHVFDETTLNNFMGL
GQAAWKEARASLQNLLSASQARLRDDKELRQRAFTSQASATMHLPATIGDYTDFYSSRQHATNVGIMFRG
KENALLPNWLHLPVGYHGRASSIVVSGTPIRRPMGQMRPDNSKPPVYGACRLLDMELEMAFFVGPGNRFG
EPIPISKAHEHIFGMVLMNDWSARDIQQWEYVPLGPFLGKSFGTTISPWVVPMDALMPFVVPNPKQDPKPL
PYLCHSQPYTFDINLSVSLKGEGMSQAATICRSNFKHMYWTMLQQLTHHSVNGCNLRPGDLLASGTISGSD
PESFGSMLELSWKGTKAIDVEQGQTRTFLLDGDEVIITGHCQGDGYRVGFGQCAGKVLPALSPA
SEQ ID NO: 22 amino acid sequence of human miniDMD protein
MLWWEEVEDCYEREDVQKKTFTKWVNAQFSKFGKQHIENLFSDLQDGRRLLDLLEGLTGQKLPKEKGST
RVHALNNVNKALRVLQNNNVDLVNIGSTDIVDGNHKLTLGLIWNIILHWQVKNVMKNIMAGLQQTNSEKI
LLSWVRQSTRNYPQVNVINFTTSWSDGLALNALIHSHRPDLFDWNSVVCQQSATQRLEHAFNIARYQLGIE
KLLDPEDVDTTYPDKKSILMYITSLFQVLPQQVSIEAIQEVEMLPRPPKVTKEEHFQLHHQMHYSQQITVSL
AQGYERTSSPKPRFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGKLSEDEETEVQEQ
MNLLNSRWECLRVASMEKQSNLHRVLMDLQNQKLKELNDWLTKTEERTRKMEEEPLGPDLEDLKRQVQ
QHKVLQEDLEQEQVRVNSLTHMVVVVDESSGDHATAALEEQLKVLGDRWANICRWTEDRWVLLQDILL
KWQRLTEEQCLFSAWLSEKEDAVNKIHTTGFKDQNEMLSSLQKLAVLKADLEKKKQSMGKLYSLKQDLL
STLKNKSVTQKTEAWLDNFARCWDNLVQKLEKSTAQETEIAVQAKQPDVEEILSKGQHLYKEKPATQPVK
RKLEDLSSEWKAVNRLLQELRAKQPDLAPGLTTIGASPTQTVTLVTQPVVTKETAISKLEMPSSLMLEVPAL
ADFNRAWTELTDWLSLLDQVIKSQRVMVGDLEDINEMIIKQKATMQDLEQRRPQLEELITAAQNLKNKTS
NQEARTIITDRIERIQNQWDEVQEHLQNRRQQLNEMLKDSTQWLEAKEEAEQVLGQARAKLESWKEGPYT
VDAIQKKITETKQLAKDLRQWQTNVDVANDLALKLLRDYSADDTRKVHMITENINASWRSIHKRVSEREA
ALEETHRLLQQFPLDLEKFLAWLTEAETTANVLQDATRKERLLEDSKGVKELMKQWQDLQGEIEAHTDV
YHNLDENSQKILRSLEGSDDAVLLQRRLDNMNFKWSELRKKSLNIRSHLEASSDQWKRLHLSLQELLVWL
QLKDDELSRQAPIGGDFPAVQKQNDVHRAFKRELKTKEPVIMSTLETVRIFLTEQPLEGLEKLYQEPRELPP
EERAQNVTRLLRKQAEEVNTEWEKLNLHSADWQRKIDETLERLRELQEATDELDLKLRQAEVIKGSWQPV
GDLLIDSLQDHLEKVKALRGEIAPLKENVSHVNDLARQLTTLGIQLSPYNLSTLEDLNTRWKLLQVAVEDR
VRQLHEAHRDFGPASQHFLSTSVQGPWERAISPNKVPYYINHETQTTCWDHPKMTELYQSLADLNNVRFS
AYRTAMKLRRLQKALCLDLLSLSAACDALDQHNLKQNDQPMDILQIINCLTTIYDRLEQEHNNLVNVPLC
VDMCLNWLLNVYDTGRTGRIRVLSFKTGIISLCKAHLEDKYRYLFKQVASSTGFCDQRRLGLLLHDSIQIPR
QLGEVASFGGSNIEPSVRSCFQFANNKPEIEAALFLDWMRLEPQSMVWLPVLHRVAAAETAKHQAKCNIC
KECPIIGFRYRSLKHFNYDICQSCFFSGRVAKGHKMHYPMVEYCTPTTSGEDVRDFAKVLKNKFRTKRYFA
KHPRMGYLPVQTVLEGDNMETPVTLINFWPVDSAPASSPQLSHDDTHSRIEHYASRLAEMENSNGSYLNDS
ISPNESIDDEHLLIQHYCQSLNQDSPLSQPRSPAQILISLESEERGELERILADLEEENRNLQAEYDRLKQQHE
HKGLSPLPSPPEMMPTSPQSPRDAELIAEAKLLRQHKGRLEARMQILEDHNKQLESQLHRLRQLLEQPQAE
AKVNGTTVSSPSTSLQRSDSSQPMLLRVVGSQTSDSMGEEDLLSPPQDTSTGLEEVMEQLNNSFPSSRGRNT
PGKPMREDTM
SEQ ID NO: 23 amino acid sequence of human DMD protein
MLWWEEVEDCYEREDVQKKTFTKWVNAQFSKFGKQHIENLFSDLQDGRRLLDLLEGLTGQKLPKEKGST
RVHALNNVNKALRVLQNNNVDLVNIGSTDIVDGNHKLTLGLIWNIILHWQVKNVMKNIMAGLQQTNSEKI
LLSWVRQSTRNYPQVNVINFTTSWSDGLALNALIHSHRPDLFDWNSVVCQQSATQRLEHAFNIARYQLGIE
KLLDPEDVDTTYPDKKSILMYITSLFQVLPQQVSIEAIQEVEMLPRPPKVTKEEHFQLHHQMHYSQQITVSL
AQGYERTSSPKPRFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGKLSEDEETEVQEQ
MNLLNSRWECLRVASMEKQSNLHRVLMDLQNQKLKELNDWLTKTEERTRKMEEEPLGPDLEDLKRQVQ
QHKVLQEDLEQEQVRVNSLTHMVVVVDESSGDHATAALEEQLKVLGDRWANICRWTEDRWVLLQDILL
KWQRLTEEQCLFSAWLSEKEDAVNKIHTTGFKDQNEMLSSLQKLAVLKADLEKKKQSMGKLYSLKQDLL
STLKNKSVTQKTEAWLDNFARCWDNLVQKLEKSTAQISQAVTTTQPSLTQTTVMETVTTVTTREQILVKH
AQEELPPPPPQKKRQITVDSEIRKRLDVDITELHSWITRSEAVLQSPEFAIFRKEGNFSDLKEKVNAIEREKAE
KFRKLQDASRSAQALVEQMVNEGVNADSIKQASEQLNSRWIEFCQLLSERLNWLEYQNNIIAFYNQLQQLE
QMTTTAENWLKIQPTTPSEPTAIKSQLKICKDEVNRLSDLQPQIERLKIQSIALKEKGQGPMFLDADFVAFTN
HFKQVFSDVQAREKELQTIFDTLPPMRYQETMSAIRTWVQQSETKLSIPQLSVTDYEIMEQRLGELQALQSS
LQEQQSGLYYLSTTVKEMSKKAPSEISRKYQSEFEEIEGRWKKLSSQLVEHCQKLEEQMNKLRKIQNHIQTL
KKWMAEVDVFLKEEWPALGDSEILKKQLKQCRLLVSDIQTIQPSLNSVNEGGQKIKNEAEPEFASRLETEL
KELNTQWDHMCQQVYARKEALKGGLEKTVSLQKDLSEMHEWMTQAEEEYLERDFEYKTPDELQKAVEE
MKRAKEEAQQKEAKVKLLTESVNSVIAQAPPVAQEALKKELETLTTNYQWLCTRLNGKCKTLEEVWACW
HELLSYLEKANKWLNEVEFKLKTTENIPGGAEEISEVLDSLENLMRHSEDNPNQIRILAQTLTDGGVMDELI
NEELETFNSRWRELHEEAVRRQKLLEQSIQSAQETEKSLHLIQESLTFIDKQLAAYIADKVDAAQMPQEAQ
KIQSDLTSHEISLEEMKKHNQGKEAAQRVLSQIDVAQKKLQDVSMKFRLFQKPANFEQRLQESKMILDEVK
MHLPALETKSVEQEVVQSQLNHCVNLYKSLSEVKSEVEMVIKTGRQIVQKKQTENPKELDERVTALKLHY
NELGAKVTERKQQLEKCLKLSRKMRKEMNVLTEWLAATDMELTKRSAVEGMPSNLDSEVAWGKATQKE
IEKQKVHLKSITEVGEALKTVLGKKETLVEDKLSLLNSNWIAVTSRAEEWLNLLLEYQKHMETFDQNVDHI
TKWIIQADTLLDESEKKKPQQKEDVLKRLKAELNDIRPKVDSTRDQAANLMANRGDHCRKLVEPQISELN
HRFAAISHRIKTGKASIPLKELEQFNSDIQKLLEPLEAEIQQGVNLKEEDFNKDMNEDNEGTVKELLQRGDN
LQQRITDERKREEIKIKQQLLQTKHNALKDLRSQRRKKALEISHQWYQYKRQADDLLKCLDDIEKKLASLP
EPRDERKIKEIDRELQKKKEELNAVRRQAEGLSEDGAAMAVEPTQIQLSKRWREIESKFAQFRRLNFAQIHT
VREETMMVMTEDMPLEISYVPSTYLTEITHVSQALLEVEQLLNAPDLCAKDFEDLFKQEESLKNIKDSLQQS
SGRIDIIHSKKTAALQSATPVERVKLQEALSQLDFQWEKVNKMYKDRQGRFDRSVEKWRRFHYDIKIFNQ
WLTEAEQFLRKTQIPENWEHAKYKWYLKELQDGIGQRQTVVRTLNATGEEIIQQSSKTDASILQEKLGSLN
LRWQEVCKQLSDRKKRLEEQKNILSEFQRDLNEFVLWLEEADNIASIPLEPGKEQQLKEKLEQVKLLVEELP
LRQGILKQLNETGGPVLVSAPISPEEQDKLENKLKQTNLQWIKVSRALPEKQGEIEAQIKDLGQLEKKLEDL
EEQLNHLLLWLSPIRNQLEIYNQPNQEGPFDVKETEIAVQAKQPDVEEILSKGQHLYKEKPATQPVKRKLED
LSSEWKAVNRLLQELRAKQPDLAPGLTTIGASPTQTVTLVTQPVVTKETAISKLEMPSSLMLEVPALADFNR
AWTELTDWLSLLDQVIKSQRVMVGDLEDINEMIIKQKATMQDLEQRRPQLEELITAAQNLKNKTSNQEAR
TIITDRIERIQNQWDEVQEHLQNRRQQLNEMLKDSTQWLEAKEEAEQVLGQARAKLESWKEGPYTVDAIQ
KKITETKQLAKDLRQWQTNVDVANDLALKLLRDYSADDTRKVHMITENINASWRSIHKRVSEREAALEET
HRLLQQFPLDLEKFLAWLTEAETTANVLQDATRKERLLEDSKGVKELMKQWQDLQGEIEAHTDVYHNLD
ENSQKILRSLEGSDDAVLLQRRLDNMNFKWSELRKKSLNIRSHLEASSDQWKRLHLSLQELLVWLQLKDD
ELSRQAPIGGDFPAVQKQNDVHRAFKRELKTKEPVIMSTLETVRIFLTEQPLEGLEKLYQEPRELPPEERAQ
NVTRLLRKQAEEVNTEWEKLNLHSADWQRKIDETLERLRELQEATDELDLKLRQAEVIKGSWQPVGDLLI
DSLQDHLEKVKALRGEIAPLKENVSHVNDLARQLTTLGIQLSPYNLSTLEDLNTRWKLLQVAVEDRVRQL
HEAHRDFGPASQHFLSTSVQGPWERAISPNKVPYYINHETQTTCWDHPKMTELYQSLADLNNVRFSAYRT
AMKLRRLOKALCLDLLSLSAACDALDQHNLKQNDQPMDILQIINCLTTIYDRLEQEHNNLVNVPLCVDMC
LNWLLNVYDTGRTGRIRVLSFKTGIISLCKAHLEDKYRYLFKQVASSTGFCDQRRLGLLLHDSIQIPRQLGE
VASFGGSNIEPSVRSCFQFANNKPEIEAALFLDWMRLEPQSMVWLPVLHRVAAAETAKHQAKCNICKECPII
GFRYRSLKHFNYDICQSCFFSGRVAKGHKMHYPMVEYCTPTTSGEDVRDFAKVLKNKFRTKRYFAKHPR
MGYLPVQTVLEGDNMETPVTLINFWPVDSAPASSPQLSHDDTHSRIEHYASRLAEMENSNGSYLNDSISPN
ESIDDEHLLIQHYCQSLNQDSPLSQPRSPAQILISLESEERGELERILADLEEENRNLQAEYDRLKQQHEHKG
LSPLPSPPEMMPTSPQSPRDAELIAEAKLLRQHKGRLEARMQILEDHNKQLESQLHRLRQLLEQPQAEAKV
NGTTVSSPSTSLQRSDSSQPMLLRVVGSQTSDSMGEEDLLSPPQDTSTGLEEVMEQLNNSFPSSRGRNTPGK
PMREDTM
SEQ ID NO: 24 amino acid sequence of human p53 protein
MEEPQSDPSVEPPLSQETFSDLWKLLPENNVLSPLPSQAMDDLMLSPDDIEQWFTEDPGPDEAPRMPEAAPP
VAPAPAAPTPAAPAPAPSWPLSSSVPSQKTYQGSYGFRLGFLHSGTAKSVTCTYSPALNKMFCQLAKTCPV
QLWVDSTPPPGTRVRAMAIYKQSQHMTEVVRRCPHHERCSDSDGLAPPQHLIRVEGNLRVEYLDDRNTFR
HSVVVPYEPPEVGSDCTTIHYNYMCNSSCMGGMNRRPILTIITLEDSSGNLLGRNSFEVRVCACPGRDRRTE
EENLRKKGEPHHELPPGSTKRALPNNTSSSPQPKKKPLDGEYFTLQIRGRERFEMFRELNEALELKDAQAGK
EPGGSRAHSSHLKSKKGQSTSRHKKLMFKTEGPDSD
SEQ ID NO: 25 amino acid sequence of human PTEN protein
MTAIIKEIVSRNKRRYQEDGFDLDLTYIYPNIIAMGFPAERLEGVYRNNIDDVVRFLDSKHKNHYKIYNLCA
ERHYDTAKFNCRVAQYPFEDHNPPQLELIKPFCEDLDQWLSEDDNHVAAIHCKAGKGRTGVMICAYLLHR
GKFLKAQEALDFYGEVRTRDKKGVTIPSQRRYVYYYSYLLKNHLDYRPVALLFHKMMFETIPMFSGGTCN
PQFVVCQLKVKIYSSNSGPTRREDKFMYFEFPQPLPVCGDIKVEFFHKQNKMLKKDKMFHFWVNTFFIPGP
EETSEKVENGSLCDQEIDSICSIERADNDKEYLVLTLTKNDLDKANKDKANRYFSPNFKVKLYFTKTVEEPS
NPEASSSTSVTPDVSDNEPDHYRYSDTTDSDPENEPFDEDQHTQITKV
SEQ ID NO: 26 amino acid sequence of SARS-CoV-2 neutralizing antibody nAB-1
QVQLVESGGGLVQAGGSLRLSCAVSGAGAHRVGWFRRAPGKEREFVAAIGASGGMTNYLDSVKGRFTISR
DNAKNTIYLQMNSLKPQDTAVYYCAARDIETAEYIYWGQGTQVTVSS
SEQ ID NO: 27 amino acid sequence of SARS-CoV-2 neutralizing antibody nAB-2
QVQLVESGGGLVQAGGSLRLSCAVSGLGAHRVGWFRRAPGKEREFVAAIGANGGNTNYLDSVKGRFTISR
DNAKNTIYLQMNSLKPQDTAVYYCAARDIETAEYTYWGQGTQVTVSS
SEQ ID NO: 28 amino acid sequence of SARS-CoV-2 neutralizing antibody nAB-3
QVQLVESGGGLVQAGGSLRLSCAVSGAGAHRVGWFRRAPGKEREFVAAIGASGGMTNYLDSVKGRFTISR
DNAKNTIYLQMNSLKPQDTAVYYCAARDIETAEYIYWGQGTQVTVSSKLGGGGSGGGGSGGGGSGGGGS
GGGGSQVQLVESGGGLVQAGGSLRLSCAVSGAGAHRVGWFRRAPGKEREFVAAIGASGGMTNYLDSVK
GRFTISRDNAKNTIYLQMNSLKPQDTAVYYCAARDIETAEYIYWGQGTQVTVSSGGGGSGGGGSGGGGSG
GGGSGGGGSQVQLVESGGGLVQAGGSLRLSCAVSGAGAHRVGWFRRAPGKEREFVAAIGASGGMTNYL
DSVKGRFTISRDNAKNTIYLQMNSLKPQDTAVYYCAARDIETAEYIYWGQGTQVTVSS
SEQ ID NO: 29 amino acid sequence of SARS-CoV-2 neutralizing antibody nAB-4
QVQLVESGGGLVQAGGSLRLSCAVSGLGAHRVGWFRRAPGKEREFVAAIGANGGNTNYLDSVKGRFTISR
DNAKNTIYLQMNSLKPQDTAVYYCAARDIETAEYTYWGQGTQVTVSSKLGGGGSGGGGSGGGGSGGGGS
GGGGSSQVQLVESGGGLVQAGGSLRLSCAVSGLGAHRVGWFRRAPGKEREFVAAIGANGGNTNYLDSVK
GRFTISRDNAKNTIYLQMNSLKPQDTAVYYCAARDIETAEYTYWGQGTQVTVSSGGGGSGGGGSGGGGS
GGGGSGGGGSQVQLVESGGGLVQAGGSLRLSCAVSGLGAHRVGWFRRAPGKEREFVAAIGANGGNTNYL
DSVKGRFTISRDNAKNTIYLQMNSLKPQDTAVYYCAARDIETAEYTYWGQGTQVTVSS
SEQ ID NO: 30 amino acid sequence of SARS-CoV-2 neutralizing antibody nAB-5
QVQLVESGGGLVQAGGSLRLSCAASGYIFGRNAMGWYRQAPGKERELVAGITRRGSITYYADSVKGRFTIS
RDNAKNTVYLQMNSLKPEDTAVYYCAADPASPAYGDYWGQGTQVTVSS
SEQ ID NO: 31 amino acid sequence of SARS-CoV-2 neutralizing antibody nAB-6
QVQLVESGGGLVQAGGSLRLSCAASGYIFGRNAMGWYRQAPGKERELVAGITRRGSITYYADSVKGRFTIS
RDNAKNTVYLQMNSLKPEDTAVYYCAADPASPAYGDYWGQGTQVTVSSGGGGSGGGGSGGGGSGGGGS
QVQLVESGGGLVQAGGSLRLSCAASGYIFGRNAMGWYRQAPGKERELVAGITRRGSITYYADSVKGRFTIS
RDNAKNTVYLQMNSLKPEDTAVYYCAADPASPAYGDYWGQGTQVTVSSGGGGSGGGGSGGGGSGGGGS
QVQLVESGGGLVQAGGSLRLSCAASGYIFGRNAMGWYRQAPGKERELVAGITRRGSITYYADSVKGRFTIS
RDNAKNTVYLQMNSLKPEDTAVYYCAADPASPAYGDYWGQGTQVTVSS
SEQ ID NO: 32 amino acid sequence of SARS-CoV-2 neutralizing antibody nAB-7H
EVQLLESGGGVVQPGGSLRLSCAASGFAFTTYAMNWVRQAPGRGLEWVSAISDGGGSAYYADSVKGRFTI
SRDNSKNTLYLQMNSLRAEDTAVYYCAKTRGRGLYDYVWGSKDYWGQGTLVTVSSASTKGPSVFPLAPS
SKSTSGGTAALGCLVKDYFPEPVTVSWNSGALTSGVHTFPAVLQSSGLYSLSSVVTVPSSSLGTQTYICNVN
HKPSNTKVDKKVEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
SEQ ID NO: 33 amino acid sequence of SARS-CoV-2 neutralizing antibody nAB-7L
DIVMTQSPLSLPVTPGEPASISCRSSQSLLHSNGYNYLDWYLQKPGQSPQLLIYLGSNRASGVPDRFSGSGSG
TDFTLKISRVEAEDVGVYYCMQALQTPGTFGQGTRLEIKRTVAAPSVFIFPPSDEQLKSGTASVVCLLNNFY
PREAKVQWKVDNALQSGNSQESVTEQDSKDSTYSLSSTLTLSKADYEKHKVYACEVTHQGLSSPVTKSFN
RGEC
SEQ ID NO: 34 amino acid sequence of ACE-binding-1
SAEIDLGKGDFREIRASEDAREAAEALAEAARAMKEALEIIREIAEKLRDSSRASEAAKRIAKAIRKAADAIA
EAAKIAARAAKDGDAARNAENAARKAKEFAEEQAKLADMYAELAKNGDKSSVLEQLKTFADKAFHEME
DRFYQAALAVFEAAEAAAGGSGWGSG
SEQ ID NO: 35 amino acid sequence of ACE-binding-2
SAEIDLGKGDFREIRASEDAREAAEALAEAARAMKEALEIIREIAEKLRDSSRASEAAKRIAKAIRKAADAIA
EAAKIAARAAKDGDAARNAENAARKAKEFAEEQAKLADMYAELAKNGDKSSVLEQLKTFADKAFHEME
DRFYQAALAVFEAAEAAAGGGGSGGSGSGGSGGGSPGSAEIDLGKGDFREIRASEDAREAAEALAEAARA
MKEALEIIREIAEKLRDSSRASEAAKRIAKAIRKAADAIAEAAKIAARAAKDGDAARNAENAARKAKEFAE
EQAKLADMYAELAKNGDKSSVLEQLKTFADKAFHEMEDRFYQAALAVFEAAEAAAGGSGWGS
SEQ ID NO: 36 Kozak nucleic acid sequence
GCCACCAUG
SEQ ID NO: 37 polyAC sequence
GAAAAACAAAAAACAAAAAAAACAAAAAAAAAACCAAAAAAACAAAACACA
SEQ ID NO: 38 m6A modification sequence
ACGAGTCCTGGACTGAAACGGACTTGT
SEQ ID NO: 39 3′ exon sequence recognizable by a 3′ catalytic Group I intron fragment
AAAAUCCGUUGACCUUAAACGGUCGUGUGGGUUCAAGUCCCUCCACCCCCAC
SEQ ID NO: 40 5′ exon sequence recognizable by a 5′ catalytic Group I intron fragment
GAGACGCUACGGACUU
SEQ ID NO: 41 Exemplary 5′ homology sequence
GGGAGACCCUCGACCGUCGAUUGUCCACUGGUC
SEQ ID NO: 42 Exemplary 3′ homology sequence
ACCAGUGGACAAUCGACGGAUAACAGCAUAUCUAG
SEQ ID NO: 43 T7 Promoter
UAAUACGACUCACUAUAGG
SEQ ID NO: 44 T2A peptide coding sequence
GAGGGCAGAGGAAGUCUUCUAACAUGCGGUGACGUGGAGGAGAAUCCCGGCCCU
SEQ ID NO: 45 P2A peptide coding sequence
GCUACUAACUUCAGCCUGCUGAAGCAGGCUGGAGACGUGGAGGAGAACCCUGGACCU
SEQ ID NO: 46 catalytic Group I intron fragment
AACAAUAGAUGACUUACAACUAAUCGGAAGGUGCAGAGACUCGACGGGAGCUACCCUAACGUCAA
GACGAGGGUAAAGAGAGAGUCCAAUUCUCAAAGCCAAUAGGCAGUAGCGAAAGCUGCAAGAGAAU
G
SEQ ID NO: 47 5′ catalytic Group I intron fragment
AAAUAAUUGAGCCUUAAAGAAGAAAUUCUUUAAGUGGAUGCUCUCAAACUCAGGGAAACCUAAAU
CUAGUUAUAGACAAGGCAAUCCUGAGCCAAGCCGAAGUAGUAAUUAGUAAG
SEQ ID NO: 48 Nucleic acid sequence of full-length S protein sequence of SARS-CoV-2
ATGTTCGTTTTCCTTGTTCTGTTGCCTCTCGTTAGTAGCCAATGCGTCAACCTTACTACTAGAACCCAGC
TCCCTCCAGCATATACCAACTCTTTCACCAGGGGCGTATATTACCCGGACAAAGTGTTCCGCTCAAGTG
TGCTGCATTCTACGCAGGACCTTTTCTTGCCCTTTTTCAGTAATGTTACTTGGTTTCATGCTATCCATGT
GTCTGGAACTAACGGAACCAAGCGCTTTGACAACCCCGTCCTCCCTTTCAACGATGGCGTGTACTTCGC
TTCCACGGAAAAGTCAAACATAATTCGCGGCTGGATCTTTGGTACAACACTCGACTCAAAGACGCAGA
GCCTGCTGATCGTTAATAACGCTACAAATGTTGTGATAAAGGTGTGTGAATTTCAGTTCTGCAATGATC
CCTTCCTGGGTGTGTACTACCATAAGAATAACAAGAGCTGGATGGAATCCGAATTTAGGGTTTACAGT
TCCGCTAACAACTGCACATTCGAATACGTAAGCCAGCCATTTCTTATGGATCTTGAGGGCAAGCAAGG
AAACTTCAAGAACTTGAGGGAGTTCGTGTTCAAAAATATCGACGGCTATTTTAAGATATATAGCAAGC
ACACTCCAATAAACTTGGTGCGCGACCTGCCCCAGGGATTCTCTGCTCTGGAGCCCCTGGTGGATCTGC
CCATTGGAATAAACATAACTCGCTTTCAAACACTGCTCGCCCTGCATCGCAGTTACCTCACCCCTGGTG
ATAGTAGTTCAGGATGGACAGCAGGAGCCGCCGCATACTACGTCGGCTACCTGCAGCCTAGGACCTTC
TTGCTGAAGTACAACGAGAACGGTACAATAACTGACGCTGTGGACTGCGCTCTGGACCCTCTGTCCGA
GACGAAGTGCACCCTGAAGAGCTTTACTGTTGAAAAAGGCATTTACCAAACCAGCAACTTCCGCGTCC
AGCCAACCGAGAGCATCGTCAGATTTCCCAACATTACAAATCTGTGTCCCTTCGGCGAGGTGTTCAAC
GCCACACGCTTCGCTTCAGTGTACGCATGGAACCGCAAGCGCATATCTAACTGCGTCGCGGATTATTCT
GTCCTCTACAACTCCGCCTCTTTCTCCACCTTCAAGTGCTACGGAGTGTCACCGACTAAGCTGAACGAT
CTCTGCTTTACCAACGTCTACGCGGACTCCTTCGTGATAAGAGGTGATGAAGTGAGACAAATAGCCCC
AGGTCAGACTGGTAAGATCGCAGATTACAACTACAAATTGCCTGATGATTTCACTGGTTGCGTTATCG
CGTGGAACTCTAATAACCTCGATTCTAAGGTCGGTGGTAACTACAATTACCTGTACCGCTTGTTTAGGA
AGTCAAACCTGAAGCCTTTCGAGAGGGATATTTCAACCGAAATCTATCAAGCGGGTTCAACACCGTGT
AACGGTGTGGAAGGATTTAACTGCTACTTCCCCCTGCAGTCTTACGGATTCCAGCCAACCAATGGCGT
GGGTTACCAACCTTATCGCGTGGTGGTTCTGAGTTTCGAACTGTTGCACGCTCCCGCCACGGTATGCGG
TCCCAAGAAGAGCACTAACTTGGTGAAGAATAAGTGCGTGAATTTCAATTTCAATGGCCTCACTGGAA
CTGGAGTGCTGACCGAATCCAATAAGAAGTTCTTGCCCTTCCAGCAGTTCGGAAGAGACATTGCTGAC
ACAACCGACGCGGTGCGCGATCCTCAGACTCTGGAGATATTGGACATTACACCATGTTCTTTCGGCGG
TGTGTCTGTCATTACTCCGGGCACGAATACTAGCAACCAGGTAGCCGTGCTGTACCAAGACGTGAATT
GCACAGAGGTTCCCGTCGCAATTCACGCTGACCAGCTGACCCCCACGTGGAGGGTTTACAGCACTGGT
AGTAACGTCTTCCAGACGAGAGCCGGTTGCTTGATCGGAGCGGAACATGTGAATAACTCCTACGAGTG
CGACATCCCCATCGGAGCCGGTATATGCGCCTCTTATCAGACACAAACTAACTCACCCAGGAGAGCCC
GCAGTGTGGCTTCTCAAAGCATTATAGCATACACTATGTCTCTTGGTGCCGAAAATTCCGTGGCCTATT
CTAACAATTCAATCGCCATCCCAACCAACTTCACAATTAGCGTGACTACCGAAATACTGCCTGTGAGC
ATGACGAAAACCAGCGTAGACTGCACTATGTATATCTGTGGAGACTCCACTGAGTGCTCCAACCTTCT
CCTGCAGTACGGTAGCTTCTGTACCCAATTGAACCGCGCCCTTACAGGCATCGCTGTTGAGCAAGATA
AGAATACCCAGGAAGTTTTTGCCCAGGTTAAGCAGATATACAAAACACCGCCCATTAAGGACTTCGGA
GGCTTCAACTTCTCTCAGATACTGCCTGACCCCTCCAAGCCATCAAAACGCAGCTTCATTGAGGACCTC
TTGTTCAACAAAGTGACTCTGGCTGATGCTGGCTTCATTAAGCAGTACGGAGATTGCCTGGGAGATAT
TGCTGCCAGGGACCTCATCTGCGCCCAGAAGTTTAATGGCCTGACAGTCTTGCCCCCACTTCTGACAGA
CGAGATGATTGCTCAGTACACATCTGCCCTCCTCGCTGGCACCATAACATCCGGATGGACATTTGGTGC
TGGTGCTGCCCTCCAGATTCCCTTCGCAATGCAGATGGCGTATCGCTTTAACGGCATCGGTGTCACACA
AAACGTGTTGTATGAGAACCAAAAGCTCATCGCTAACCAGTTTAATTCTGCTATTGGTAAGATTCAGG
ACAGCCTGTCATCAACCGCGTCTGCCCTTGGTAAGTTGCAGGACGTGGTGAACCAGAATGCTCAGGCT
TTGAATACTCTGGTGAAGCAACTCTCTTCAAATTTCGGCGCTATCTCTTCTGTGTTGAACGACATCCTG
AGTCGCCTTGATAAGGTGGAAGCTGAAGTTCAAATTGATAGATTGATTACTGGCAGGCTCCAGTCTTT
GCAGACCTACGTTACACAGCAGCTGATTAGGGCGGCTGAAATTAGAGCTTCCGCCAATCTGGCTGCAA
CCAAGATGTCCGAATGCGTCCTGGGTCAGTCAAAGCGCGTTGACTTTTGTGGTAAAGGCTACCACCTC
ATGTCATTTCCCCAGTCAGCACCTCACGGAGTAGTGTTCCTCCACGTCACCTACGTTCCAGCACAGGAA
AAGAATTTTACCACTGCGCCGGCAATCTGTCACGACGGTAAGGCACACTTCCCCCGCGAGGGCGTATT
CGTGTCTAACGGAACTCATTGGTTCGTCACACAGAGAAACTTCTATGAGCCTCAGATCATTACCACCG
ACAATACATTTGTGTCCGGTAACTGCGACGTTGTGATTGGAATCGTCAACAACACTGTGTACGATCCA
CTTCAGCCAGAACTGGATAGCTTCAAGGAAGAATTGGACAAATATTTCAAAAATCACACTTCACCCGA
TGTGGACCTGGGTGACATTAGTGGTATCAATGCGTCCGTGGTCAATATTCAAAAAGAGATTGACAGGC
TCAACGAAGTGGCCAAGAACCTGAACGAAAGTCTTATCGATCTGCAAGAATTGGGAAAGTATGAGCA
GTACATCAAGTGGCCGTGGTACATTTGGTTGGGTTTTATCGCCGGTCTGATCGCCATCGTTATGGTTAC
CATTATGCTTTGCTGCATGACGAGCTGTTGCTCCTGTCTGAAGGGATGCTGCTCTTGCGGATCATGTTG
CAAGTTCGATGAAGACGATAGCGAACCAGTTCTGAAGGGCGTCAAGCTGCATTACACA
SEQ ID NO: 49 Nucleic acid sequence of the RBD amino acid residues 319-542 of S protein
CGCGTCCAGCCAACCGAGAGCATCGTCAGATTTCCCAACATTACAAATCTGTGTCCCTTCGGCGAGGT
GTTCAACGCCACACGCTTCGCTTCAGTGTACGCATGGAACCGCAAGCGCATATCTAACTGCGTCGCGG
ATTATTCTGTCCTCTACAACTCCGCCTCTTTCTCCACCTTCAAGTGCTACGGAGTGTCACCGACTAAGCT
GAACGATCTCTGCTTTACCAACGTCTACGCGGACTCCTTCGTGATAAGAGGTGATGAAGTGAGACAAA
TAGCCCCAGGTCAGACTGGTAAGATCGCAGATTACAACTACAAATTGCCTGATGATTTCACTGGTTGC
GTTATCGCGTGGAACTCTAATAACCTCGATTCTAAGGTCGGTGGTAACTACAATTACCTGTACCGCTTG
TTTAGGAAGTCAAACCTGAAGCCTTTCGAGAGGGATATTTCAACCGAAATCTATCAAGCGGGTTCAAC
ACCGTGTAACGGTGTGGAAGGATTTAACTGCTACTTCCCCCTGCAGTCTTACGGATTCCAGCCAACCA
ATGGCGTGGGTTACCAACCTTATCGCGTGGTGGTTCTGAGTTTCGAACTGTTGCACGCTCCCGCCACGG
TATGCGGTCCCAAGAAGAGCACTAACTTGGTGAAGAATAAGTGCGTGAATTTC
SEQ ID NO: 50 Nucleic acid sequence of the C-terminal Foldon domain of a T4 fibritin protein
GGAAGCGGCTACATCCCAGAAGCCCCTAGAGACGGACAGGCTTACGTGCGAAAAGACGGCGAGTGGG
TGCTGCTGAGCACATTCCTGGGAAGGAGC
SEQ ID NO: 51 Nucleic acid sequence of the GCN4-based isoleucine zipper domain
CGAATGAAGCAGATTGAGGATAAAATTGAGGAGATTCTCAGCAAAATTTACCACATAGAAAATGAGA
TCGCTCGGATTAAAAAACTGATCGGAGAAAGA
SEQ ID NO: 52 Nucleic acid sequence of the GS peptide linker
GGCGGAGGAGGCAGCGGCGGAGGAGGCAGC
SEQ ID NO: 53 CVB3 virus IRES
TTAAAACAGCCTGTGGGTTGATCCCACCCACAGGCCCATTGGGCGCTAGCACTCTGGTATCACGGTAC
CTTTGTGCGCCTGTTTTATACCCCCTCCCCCAACTGTAACTTAGAAGTAACACACACCGATCAACAGTC
AGCGTGGCACACCAGCCACGTTTTGATCAAGCACTTCTGTTACCCCGGACTGAGTATCAATAGACTGC
TCACGCGGTTGAAGGAGAAAGCGTTCGTTATCCGGCCAACTACTTCGAAAAACCTAGTAACACCGTGG
AAGTTGCAGAGTGTTTCGCTCAGCACTACCCCAGTGTAGATCAGGTCGATGAGTCACCGCATTCCCCA
CGGGCGACCGTGGCGGTGGCTGCGTTGGCGGCCTGCCCATGGGGAAACCCATGGGACGCTCTAATACA
GACATGGTGCGAAGAGTCTATTGAGCTAGTTGGTAGTCCTCCGGCCCCTGAATGCGGCTAATCCTAAC
TGCGGAGCACACACCCTCAAGCCAGAGGGCAGTGTGTCGTAACGGGCAACTCTGCAGCGGAACCGAC
TACTTTGGGTGTCCGTGTTTCATTTTATTCCTATACTGGCTGCTTATGGTGACAATTGAGAGATCGTTAC
CATATAGCTATTGGATTGGCCATCCGGTGACTAATAGAGCTATTATATATCCCTTTGTTGGGTTTATAC
CACTTAGCTTGAAAGAGGTTAAAACATTACAATTCATTGTTAAGTTGAATACAGCAAA
SEQ ID NO: 54 amino acid sequence of human Fumarylacetoacetase (FAH) protein
MSFIPVAEDSDFPIHNLPYGVFSTRGDPRPRIGVAIGDQILDLSIIKHLFTGPVLSKHQDVFNQPTLNSFMGLG
QAAWKEARVFLQNLLSVSQARLRDDTELRKCAFISQASATMHLPATIGDYTDFYSSRQHATNVGIMFRDK
ENALMPNWLHLPVGYHGRASSVVVSGTPIRRPMGQMKPDDSKPPVYGACKLLDMELEMAFFVGPGNRLG
EPIPISKAHEHIFGMVLMNDWSARDIQKWEYVPLGPFLGKSFGTTVSPWVVPMDALMPFAVPNPKQDPRPL
PYLCHDEPYTFDINLSVNLKGEGMSQAATICKSNFKYMYWTMLQQLTHHSVNGCNLRPGDLLASGTISGP
EPENFGSMLELSWKGTKPIDLGNGQTRKFLLDGDEVIITGYCQGDGYRIGFGQCAGKVLPALLPS
SEQ ID NO: 55 amino acid sequence of human Ornithine carbamoyltransferase (OTC) protein
MLFNLRILLNNAAFRNGHNFMVRNFRCGQPLQNKVQLKGRDLLTLKNFTGEEIKYMLWLSADLKFRIKQK
GEYLPLLQGKSLGMIFEKRSTRTRLSTETGLALLGGHPCFLTTQDIHLGVNESLTDTARVLSSMADAVLARV
YKQSDLDTLAKEASIPIINGLSDLYHPIQILADYLTLQEHYSSLKGLTLSWIGDGNNILHSIMMSAAKFGMHL
QAATPKGYEPDASVTKLAEQYAKENGTKLLLTNDPLEAAHGGNVLITDTWISMGQEEEKKKRLQAFQGY
QVTMKTAKVAASDWTFLHCLPRKPEEVDDEVFYSPRSLVFPEAENRKWTIMAVMVSLLTDYSPQLQKPKF
SEQ ID NO: 56 amino acid sequence of human Ornithine COL3A1 protein
MMSFVQKGSWLLLALLHPTIILAQQEAVEGGCSHLGQSYADRDVWKPEPCQICVCDSGSVLCDDIICDDQE
LDCPNPEIPFGECCAVCPQPPTAPTRPPNGQGPQGPKGDPGPPGIPGRNGDPGIPGQPGSPGSPGPPGICESCP
TGPQNYSPQYDSYDVKSGVAVGGLAGYPGPAGPPGPPGPPGTSGHPGSPGSPGYQGPPGEPGQAGPSGPPG
PPGAIGPSGPAGKDGESGRPGRPGERGLPGPPGIKGPAGIPGFPGMKGHRGFDGRNGEKGETGAPGLKGEN
GLPGENGAPGPMGPRGAPGERGRPGLPGAAGARGNDGARGSDGQPGPPGPPGTAGFPGSPGAKGEVGPAG
SPGSNGAPGQRGEPGPQGHAGAQGPPGPPGINGSPGGKGEMGPAGIPGAPGLMGARGPPGPAGANGAPGL
RGGAGEPGKNGAKGEPGPRGERGEAGIPGVPGAKGEDGKDGSPGEPGANGLPGAAGERGAPGFRGPAGP
NGIPGEKGPAGERGAPGPAGPRGAAGEPGRDGVPGGPGMRGMPGSPGGPGSDGKPGPPGSQGESGRPGPP
GPSGPRGQPGVMGFPGPKGNDGAPGKNGERGGPGGPGPQGPPGKNGETGPQGPPGPTGPGGDKGDTGPPG
PQGLQGLPGTGGPPGENGKPGEPGPKGDAGAPGAPGGKGDAGAPGERGPPGLAGAPGLRGGAGPPGPEGG
KGAAGPPGPPGAAGTPGLQGMPGERGGLGSPGPKGDKGFPGGPGADGVPGKDGPRGPTGPIGPPGPAGQP
GDKGEGGAPGLPGIAGPRGSPGERGETGPPGPAGFPGAPGQNGEPGGKGERGAPGEKGEGGPPGVAGPPG
KDGTSGHPGPIGPPGPRGNRGERGSEGSPGHPGQPGPPGPPGAPGPCCGGVGAAAIAGIGGEKAGGFAPYY
GDEPMDFKINTDEIMTSLKSVNGQIESLISPDGSRKNPARNCRDLKFCHPELKSGEYWVDPNQGCKLDAIKV
FCNMETGETCISANPLNVPRKHWWTDSSAEKKHVWFGESMDGGFQFSYGNPELPEDVLDVQLAFLRLLSS
RASQNITYHCKNSIAYMDQASGNVKKALKLMGSNEGEFKAEGNSKFTYTVLEDGCTKHTGEWSKTVFEY
RTRKAVRLPIVDIAPYDIGGPDQEFGVDVGPVCFL
SEQ ID NO: 57 amino acid sequence of human BMPR2 protein
MTSSLQRPWRVPWLPWTILLVSTAAASQNQERLCAFKDPYQQDLGIGESRISHENGTILCSKGSTCYGLWE
KSKGDINLVKQGCWSHIGDPQECHYEECVVTTTPPSIQNGTYRFCCCSTDLCNVNFTENFPPPDTTPLSPPHS
FNRDETIIIALASVSVLAVLIVALCFGYRMLTGDRKQGLHSMNMMEAAASEPSLDLDNLKLLELIGRGRYG
AVYKGSLDERPVAVKVFSFANRQNFINEKNIYRVPLMEHDNIARFIVGDERVTADGRMEYLLVMEYYPNG
SLCKYLSLHTSDWVSSCRLAHSVTRGLAYLHTELPRGDHYKPAISHRDLNSRNVLVKNDGTCVISDFGLSM
RLTGNRLVRPGEEDNAAISEVGTIRYMAPEVLEGAVNLRDCESALKQVDMYALGLIYWEIFMRCTDLFPGE
SVPEYQMAFQTEVGNHPTFEDMQVLVSREKQRPKFPEAWKENSLAVRSLKETIEDCWDQDAEARLTAQC
AEERMAELMMIWERNKSVSPTVNPMSTAMQNERNLSHNRRVPKIGPYPDYSSSSYIEDSIHHTDSIVKNISS
EHSMSSTPLTIGEKNRNSINYERQQAQARIPSPETSVTSLSTNTTTTNTTGLTPSTGMTTISEMPYPDETNLHT
TNVAQSIGPTPVCLQLTEEDLETNKLDPKEVDKNLKESSDENLMEHSLKQFSGPDPLSSTSSSLLYPLIKLAV
EATGQQDFTQTANGQACLIPDVLPTQIYPLPKQQNLPKRPTSLPLNTKNSTKEPRLKFGSKHKSNLKQVETG
VAKMNTINAAEPHVVTVTMNGVAGRNHSVNSHAATTQYANGTVLSGQTTNIVTHRAQEMLQNQFIGEDT
RLNINSSPDEHEPLLRREQQAGHDEGVLDRLVDRRERPLEGGRTNSNNNNSNPCSEQDVLAQGVPSTAADP
GPSKPRRAQRPNSLDLSATNVLDGSSIQIGESTQDGKSGSGEKIKKRVKTPYSLKRWRPSTWVISTESLDCE
VNNNGSNRAVHSKSSTAVYLAEGGTATTMVSKDIGMNCL
SEQ ID NO: 58 amino acid sequence of human AHI1 protein
MPTAESEAKVKTKVRFEELLKTHSDLMREKKKLKKKLVRSEENISPDTIRSNLHYMKETTSDDPDTIRSNLP
HIKETTSDDVSAANTNNLKKSTRVTKNKLRNTQLATENPNGDASVEEDKQGKPNKKVIKTVPQLTTQDLK
PETPENKVDSTHQKTHTKPQPGVDHQKSEKANEGREETDLEEDEELMQAYQCHVTEEMAKEIKRKIRKKL
KEQLTYFPSDTLFHDDKLSSEKRKKKKEVPVFSKAETSTLTISGDTVEGEQKKESSVRSVSSDSHQDDEISS
MEQSTEDSMQDDTKPKPKKTKKKTKAVADNNEDVDGDGVHEITSRDSPVYPKCLLDDDLVLGVYIHRTD
RLKSDFMISHPMVKIHVVDEHTGQYVKKDDSGRPVSSYYEKENVDYILPIMTQPYDFKQLKSRLPEWEEQI
VFNENFPYLLRGSDESPKVILFFEILDFLSVDEIKNNSEVQNQECGFRKIAWAFLKLLGANGNANINSKLRLQ
LYYPPTKPRSPLSVVEAFEWWSKCPRNHYPSTLYVVRGLKVPDCIKPSYRSMMAPQEEKGKPVHCERHHE
SSSVDTEPGLEESKEVIKWKRLPGQACRIPNKHLFSLNAGERGCFCLDFSHNGRILAAACASRDGYPIILYEIP
SGRFMRELCGHLNIIYDLSWSKDDHYILTSSSDGTARIWKNEINNTNTFRVLPHPSFVYTAKFHPAVRELVV
TGCYDSMIRIWKVEMREDSAILVRQFDVHKSFINSLCFDTEGHHMYSGDCTGVIVVWNTYVKINDLEHSV
HHWTINKEIKETEFKGIPISYLEIHPNGKRLLIHTKDSTLRIMDLRILVARKFVGAANYREKIHSTLTPCGTFL
FAGSEDGIVYVWNPETGEQVAMYSDLPFKSPIRDISYHPFENMVAFCAFGQNEPILLYIYDFHVAQQEAEMF
KRYNGTFPLPGIHQSQDALCTCPKLPHQGSFQIDEFVHTESSSTKMQLVKQRLETVTEVIRSCAAKVNKNLS
FTSPPAVSSQQSKLKQSNMLTAQEILHQFGFTQTGIISIERKPCNHQVDTAPTVVALYDYTANRSDELTIHRG
DIIRVFFKDNEDWWYGSIGKGQEGYFPANHVASETLYQELPPEIKERSPPLSPEEKTKIEKSPAPQKQSINKN
KSQDFRLGSESMTHSEMRKEQSHEDQGHIMDTRMRKNKQAGRKVTLIE
SEQ ID NO: 59 amino acid sequence of human FANCC protein
MAQDSVDLSCDYQFWMQKLSVWDQASTLETQQDTCLHVAQFQEFLRKMYEALKEMDSNTVIERFPTIGQ
LLAKACWNPFILAYDESQKILIWCLCCLINKEPQNSGQSKLNSWIQGVLSHILSALRFDKEVALFTQGLGYA
PIDYYPGLLKNMVLSLASELRENHLNGFNTQRRMAPERVASLSRVCVPLITLTDVDPLVEALLICHGREPQE
ILQPEFFEAVNEAILLKKISLPMSAVVCLWLRHLPSLEKAMLHLFEKLISSERNCLRRIECFIKDSSLPQAACH
PAIFRVVDEMFRCALLETDGALEIIATIQVFTQCFVEALEKASKQLRFALKTYFPYTSPSLAMVLLQDPQDIP
RGHWLQTLKHISELLREAVEDQTHGSCGGPFESWFLFIHFGGWAEMVAEQLLMSAAEPPTALLWLLAFYY
GPRDGRQQRAQTMVQVKAVLGHLLAMSRSSSLSAQDLQTVAGQGTDTDLRAPAQQLIRHLLLNFLLWAP
GGHTIAWDVITLMAHTAEITHEIIGFLDQTLYRWNRLGIESPRSEKLARELLKELRTQV
SEQ ID NO: 60 amino acid sequence of human MYBPC3 protein
MPEPGKKPVSAFSKKPRSVEVAAGSPAVFEAETERAGVKVRWQRGGSDISASNKYGLATEGTRHTLAVRE
VGPADQGSYAVIAGSSKVKFDLKVIEAEEAEPMLAPAPAPAEATGAPGEAPAPAAELGESAPSPKGSSSAAL
NGPTPGAPDDPIGLFVMRPQDGEVTVGGSITFSARVAGASLLKPPVVKWFKGKWVDLSSKVGQHLQLHDS
YDRASKVYLFELHITDAQPAFTGSYRCEVSTKDKFDCSNFNLTVHEAMGTGDLDLLSAFRRTSLAGGGRRI
SDSHEDTGILDFSSLLKKRDSFRTPRDSKLEAPAEEDVWETLRQAPPSEYERIAFQYGVTDLRGMLKRLKG
MRRDEKKSTAFQKKLEPAYQVSKGHKIRLTVELADHDAEVKWLKDGQEIQMSGSKYIFESIGAKRTLTISQ
CSLADDAAYQCVVGGEKCSTELFVKEPPVLITRPLEDQLVMVGQRVEFECEVSEEGAQVKWLKDGVELTR
EETFKYRFKKDGQRHHLIINEAMLEDAGHYALCTSGGQALAELIVQEKKLEVYQSIADLMVGAKDQAVFK
CEVSDENVRGVWLKNGKELVPDSRIKVSHIGRVHKLTIDDVTPADEADYSFVPEGFACNLSAKLHFMEVKI
DFVPRQEPPKIHLDCPGRIPDTIVVVAGNKLRLDVPISGDPAPTVIWQKAITQGNKAPARPAPDAPEDTGDS
DEWVFDKKLLCETEGRVRVETTKDRSIFTVEGAEKEDEGVYTVTVKNPVGEDQVNLTVKVIDVPDAPAAP
KISNVGEDSCTVQWEPPAYDGGQPILGYILERKKKKSYRWMRLNFDLIQELSHEARRMIEGVVYEMRVYA
VNAIGMSRPSPASQPFMPIGPPSEPTHLAVEDVSDTTVSLKWRPPERVGAGGLDGYSVEYCPEGCSEWVAA
LQGLTEHTSILVKDLPTGARLLSRVRAHNMAGPGAPVTTTEPVTVQEILQRPRLQLPRHLRQTIQKKVGEPV
NLLIPFQGKPRPQVTWTKEGQPLAGEEVSIRNSPTDTILFIRAARRVHSGTYQVTVRIENMEDKATLVLQVV
DKPSPPQDLRVTDAWGLNVALEWKPPQDVGNTELWGYTVQKADKKTMEWFTVLEHYRRTHCVVPELIIG
NGYYFRVFSQNMVGFSDRAATTKEPVFIPRPGITYEPPNYKALDFSEAPSFTQPLVNRSVIAGYTAMLCCAV
RGSPKPKISWFKNGLDLGEDARFRMFSKQGVLTLEIRKPCPFDGGIYVCRATNLQGEARCECRLEVRVPQ
SEQ ID NO: 61 amino acid sequence of human IL2RG protein
MLKPSLPFTSLLFLQLPLLGVGLNTTILTPNGNEDTTADFFLTTMPTDSLSVSTLPLPEVQCFVFNVEYMNCT
WNSSSEPQPTNLTLHYWYKNSDNDKVQKCSHYLFSEEITSGCQLQKKEIHLYQTFVVQLQDPREPRRQATQ
MLKLQNLVIPWAPENLTLHKLSESQLELNWNNRFLNHCLEHLVQYRTDWDHSWTEQSVDYRHKFSLPSV
DGQKRYTFRVRSRFNPLCGSAQHWSEWSHPIHWGSNTSKENPFLFALEAVVISVGSMGLIISLLCVYFWLE
RTMPRIPTLKNLEDLVTEYHGNFSAWSGVSKGLAESLQPDYSERLCLVSEIPPKGGALGEGPGASPCNQHSP
YWAPPCYTLKPET
SEQ ID NO: 62 amino acid residues 2-1273 sequence of S protein of SARS-CoV-2, K986P V987P
FVFLVLLPLVSSQCVNLTTRTQLPPAYTNSFTRGVYYPDKVFRSSVLHSTQDLFLPFFSNVTWFHAIHVSGT
NGTKRFDNPVLPFNDGVYFASTEKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFKNIDGYFKIYSKHTPINLVRDL
PQGFSALEPLVDLPIGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRFASVYAWNRKRISNC
VADYSVLYNSASFSTFKCYGVSPTKLNDLCFTNVYADSFVIRGDEVRQIAPGQTGKIADYNYKLPDDFTGC
VIAWNSNNLDSKVGGNYNYLYRLFRKSNLKPFERDISTEIYQAGSTPCNGVEGFNCYFPLQSYGFQPTNGV
GYQPYRVVVLSFELLHAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPTWRVYSTGSNVFQTR
AGCLIGAEHVNNSYECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQEVFAQVKQIYKTP
PIKDFGGFNFSQILPDPSKPSKRSFIEDLLFNKVTLADAGFIKQYGDCLGDIAARDLICAQKFNGLTVLPPLLT
DEMIAQYTSALLAGTITSGWTFGAGAALQIPFAMQMAYRFNGIGVTQNVLYENQKLIANQFNSAIGKIQDS
LSSTASALGKLQDVVNQNAQALNTLVKQLSSNFGAISSVLNDILSRLDPPEAEVQIDRLITGRLQSLQTYVT
QQLIRAAEIRASANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHYT
SEQ ID NO: 63 amino acid sequence of SARS-CoV-2 strain B.1.351 RBD
RVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGNIADYNYKLPDDFTGCVIAWNSNNLDSKVGGNYNYLYRLFRKSN
LKPFERDISTEIYQAGSTPCNGVKGFNCYFPLQSYGFQPTYGVGYQPYRVVVLSFELLHAPATVCGPKKSTN
LVKNKCVNF
SEQ ID NO: 64 Nucleic acid sequence encoding SARS-CoV-2 strain B.1.351 RBD
CGCGTCCAGCCAACCGAGAGCATCGTCAGATTTCCCAACATTACAAATCTGTGTCCCTTCGGCGAGGT
GTTCAACGCCACACGCTTCGCTTCAGTGTACGCATGGAACCGCAAGCGCATATCTAACTGCGTCGCGG
ATTATTCTGTCCTCTACAACTCCGCCTCTTTCTCCACCTTCAAGTGCTACGGAGTGTCACCGACTAAGCT
GAACGATCTCTGCTTTACCAACGTCTACGCGGACTCCTTCGTGATAAGAGGTGATGAAGTGAGACAAA
TAGCCCCAGGTCAGACTGGTAACATCGCAGATTACAACTACAAATTGCCTGATGATTTCACTGGTTGC
GTTATCGCGTGGAACTCTAATAACCTCGATTCTAAGGTCGGTGGTAACTACAATTACCTGTACCGCTTG
TTTAGGAAGTCAAACCTGAAGCCTTTCGAGAGGGATATTTCAACCGAAATCTATCAAGCGGGTTCAAC
ACCGTGTAACGGTGTGAAAGGATTTAACTGCTACTTCCCCCTGCAGTCTTACGGATTCCAGCCAACCTA
TGGCGTGGGTTACCAACCTTATCGCGTGGTGGTTCTGAGTTTCGAACTGTTGCACGCTCCCGCCACGGT
ATGCGGTCCCAAGAAGAGCACTAACTTGGTGAAGAATAAGTGCGTGAATTTC
SEQ ID NO: 96 Amino acid sequence of SARS-CoV-2 Delta variant RBD
RVQPTESIVRFPNITNLCPFGEVFNATRFASVYAWNRKRISNCVADYSVLYNSASFSTFKCYGVSPTKLNDL
CFTNVYADSFVIRGDEVRQIAPGQTGKIADYNYKLPDDFTGCVIAWNSNNLDSKVGGNYNYRYRLFRKSN
LKPFERDISTEIYQAGSKPCNGVEGFNCYFPLQSYGFQPTNGVGYQPYRVVVLSFELLHAPATVCGPKKSTN
LVKNKCVNF
SEQ ID NO: 97 Amino acid sequence of SARS-CoV-2 Omicron variant RBD
RVQPTESIVRFPNITNLCPFDEVFNATRFASVYAWNRKRISNCVADYSVLYNLAPFFTFKCYGVSPTKLNDL
CFTNVYADSFVIRGDEVRQIAPGQTGNIADYNYKLPDDFTGCVIAWNSNKLDSKVSGNYNYLYRLFRKSNL
KPFERDISTEIYQAGNKPCNGVAGFNCYFPLRSYSFRPTYGVGHQPYRVVVLSFELLHAPATVCGPKKSTNL
VKNKCVNF
SEQ ID NO: 98 Nucleic acid sequence of SARS-CoV-2 Delta variant RBD
CGCGTCCAGCCAACCGAGAGCATCGTCAGATTTCCCAACATTACAAATCTGTGTCCCTTCGGCGAGGT
GTTCAACGCCACACGCTTCGCTTCAGTGTACGCATGGAACCGCAAGCGCATATCTAACTGCGTCGCGG
ATTATTCTGTCCTCTACAACTCCGCCTCTTTCTCCACCTTCAAGTGCTACGGAGTGTCACCGACTAAGCT
GAACGATCTCTGCTTTACCAACGTCTACGCGGACTCCTTCGTGATAAGAGGTGATGAAGTGAGACAAA
TAGCCCCAGGTCAGACTGGTAAGATCGCAGATTACAACTACAAATTGCCTGATGATTTCACTGGTTGC
GTTATCGCGTGGAACTCTAATAACCTCGATTCTAAGGTCGGTGGTAACTACAATTACaGGTACCGCTTG
TTTAGGAAGTCAAACCTGAAGCCTTTCGAGAGGGATATTTCAACCGAAATCTATCAAGCGGGTTCAAag
CCGTGTAACGGTGTGgAAGGATTTAACTGCTACTTCCCCCTGCAGTCTTACGGATTCCAGCCAACCAAT
GGCGTGGGTTACCAACCTTATCGCGTGGTGGTTCTGAGTTTCGAACTGTTGCACGCTCCCGCCACGGTA
TGCGGTCCCAAGAAGAGCACTAACTTGGTGAAGAATAAGTGCGTGAATTTC
SEQ ID NO: 99 Nucleic acid sequence of SARS-CoV-2 Omicron variant RBD
CGCGTCCAGCCAACCGAGAGCATCGTCAGATTTCCCAACATTACAAATCTGTGTCCCTTCGaCGAGGTG
TTCAACGCCACACGCTTCGCTTCAGTGTACGCATGGAACCGCAAGCGCATATCTAACTGCGTCGCGGA
TTATTCTGTCCTCTACAACctCGCCcCTTTCTtCACCTTCAAGTGCTACGGAGTGTCACCGACTAAGCTGA
ACGATCTCTGCTTTACCAACGTCTACGCGGACTCCTTCGTGATAAGAGGTGATGAAGTGAGACAAATA
GCCCCAGGTCAGACTGGTAAcATCGCAGATTACAACTACAAATTGCCTGATGATTTCACTGGTTGCGTT
ATCGCGTGGAACTCTAATAAgCTCGATTCTAAGGTCaGTGGTAACTACAATTACCTGTACCGCTTGTTTA
GGAAGTCAAACCTGAAGCCTTTCGAGAGGGATATTTCAACCGAAATCTATCAAGCGGGTaacAagCCGT
GTAACGGTGTGgcAGGATTTAACTGCTACTTCCCCCTGCgGTCTTACaGcTTCCgGCCAACCtATGGCGTG
GGTcACCAACCTTATCGCGTGGTGGTTCTGAGTTTCGAACTGTTGCACGCTCCCGCCACGGTATGCGGT
CCCAAGAAGAGCACTAACTTGGTGAAGAATAAGTGCGTGAATTTC

What is claimed is:

1. A method of treating or preventing a disease or condition in an individual, comprising administering to the individual an effective amount of a circular RNA (circRNA) comprising a nucleic acid sequence encoding a therapeutic polypeptide, wherein the circRNA is a naked circRNA.

2. The method of claim 1, wherein the disease or condition is an infection.

3. The method of claim 2, wherein the infection is a coronavirus infection.

4. (canceled)

5. The method of claim 1, wherein the disease or condition is a disease or condition associated with insufficient level and/or activity of a protein corresponding to the therapeutic polypeptide, or wherein the disease or condition is a hereditary genetic disease associated with one or more mutations in the protein corresponding to the therapeutic polypeptide.

6. (canceled)

7. The method of claim 5, wherein:
(i) the therapeutic polypeptide is TP53 or PTEN, and the disease or condition is cancer;
(ii) the therapeutic polypeptide is ornithine carbamoyltransferase (OTC), and the disease or condition is ornithine transcarbamylase deficiency;
(iii) the therapeutic polypeptide is fumarylacetoacetase (FAH), and the disease or condition is tyrosinemia;
(iv) the therapeutic polypeptide is DMD, and the disease or condition is Duchenne and Becker muscular dystrophy, X-linked dilated cardiomyopathy, or familial dilated cardiomyopathy;
(v) the therapeutic polypeptide is α-l-iduronidase (IDUA), and the disease or condition is Mucopolysaccharidosis type I (MPS I);
(vi) the therapeutic polypeptide is COL3A1, and the disease or condition is Ehlers-Danlos syndrome;
(vii) the therapeutic polypeptide is AHI1, and the disease or condition is Joubert syndrome;
(viii) the therapeutic polypeptide is BMPR2, and the disease or condition is pulmonary arterial hypertension or pulmonary veno-occlusive disease;
(ix) the therapeutic polypeptide is FANCC, and the disease or condition is Fanconi anemia;
(x) the therapeutic polypeptide is MYBPC3, and the disease or condition is primary familial hypertrophic cardiomyopathy; or
(xi) the therapeutic polypeptide is IL2RG, and the disease or condition is X-linked severe combined immunodeficiency.

8. The method of claim 1, wherein the circRNA is subject to rolling circle translation by a ribosome in the individual.

9. The method of claim 1, wherein the circRNA is administered two or more times to the individual, wherein the interval between each administration is at least about four weeks.

10. (canceled)

11. The method of claim 1, wherein the circRNA is formulated as a solution.

12. (canceled)

13. The method of claim 11, wherein the solution is substantially free of adjuvant.

14. The method of claim 11, wherein the solution further comprises an adjuvant.

15. The method of claim 14, wherein the solution is substantially free of aluminum hydroxide.

16. The method of claim 1, wherein the circRNA is administered intravenously, intramuscularly, subcutaneously, transdermally, or via lymph node.

17. The method of claim 1 wherein the circRNA is administered at a dose of about 1 μg to about 10000 μg.

18.-21. (canceled)

22. The method of claim 1, wherein the circRNA further comprises an internal ribosomal entry site (IRES) sequence and a polyAC or polyA sequence disposed at the 5′ end of the IRES sequence.

23. The method of claim 1, wherein the circRNA further comprises an m6A modification motif sequence operably linked to the nucleic acid sequence encoding the therapeutic polypeptide.

24.-26. (canceled)

27. The method of claim 5, wherein the antigenic polypeptide comprises a Spike(S) protein or a fragment thereof of a coronavirus.

28.-33. (canceled)

34. The method of claim 5, wherein the therapeutic polypeptide is a receptor protein and wherein the receptor protein is an ACE2 receptor.

35.-36. (canceled)

37. The method of claim 5, wherein the therapeutic polypeptide is an antibody.

38.-39. (canceled)

40. The method of claim 5, wherein the therapeutic polypeptide is a functional protein.

41.-43. (canceled)

44. The method of claim 1, wherein the method comprises administering to the individual a plurality of circRNAs of claim 1, wherein the therapeutic polypeptides encoded by the plurality of circRNAs are different with respect to each other.

45. A pharmaceutical composition comprising the circRNA recited in claim 1, wherein the pharmaceutical composition is not formulated with a transfection agent.
